SEQUENCE LISTING (10 sequences)

SEQ ID NO: 1 - protein, 25 aa, synthetic construct [sequence omitted]

SEQ ID NO: 2 - protein, 17 aa, synthetic construct
MGWFRQAPGKGRELVAA

SEQ ID NO: 3 - protein, 38 aa, synthetic construct
YPDSVEGRFTISRDNAKRMVYLQMNSLRAEDTAVYYCA

SEQ ID NO: 4 - protein, 11 aa, synthetic construct
WGQGTQVTVSS

SEQ ID NO: 5 - protein, 121 aa, synthetic construct
EVQLVESGGGLVQPGGSLRLSCAASGFSSIVYMGWFRQAPGKGRELVAAIDASGSTTNYPDSVEGRFTISRDNAKRMVYLQMNSLRAEDTAVYYCAIAYFTSPEYVVSQGWGQGTQVTVSS

SEQ ID NO: 6 - protein, 23 aa, synthetic construct
GQAGQHHHHHHGAYPYDVPDYAS

SEQ ID NO: 7-8 - skipped (2 entries)

SEQ ID NO: 9 - protein, 15 aa, synthetic construct
GGGGSGGGGSGGGGS

SEQ ID NO: 10 - protein, 4 aa, synthetic construct
GSAS

(19) United States
(12) Patent Application Publication
(10) Pub. No.: US 2024/0401239 A1
(43) Pub. Date: Dec. 5, 2024

(54) SYNTHETIC HUMANIZED LLAMA NANOBODY LIBRARY AND USE THEREOF TO IDENTIFY SARS-COV-2 NEUTRALIZING ANTIBODIES

(71) Applicant: The U.S.A., as represented by The Secretary, Department of Health and Human Services, Bethesda, MD (US)

(72) Inventors: Ying Fu, Rockville, MD (US); Bryan D. Fleming, Gaithersburg, MD (US); Alex Renn, Rockville, MD (US); Matthew D. Hall, Darnestown, MD (US); Anton Simeonov, Bethesda, MD (US)

(73) Assignee: The U.S.A., as represented by The Secretary, Department of Health and Human Services, Bethesda, MD (US)

(21) Appl. No.: 18/692,570

(22) PCT Filed: Sep. 9, 2022
(86) PCT No.: PCT/US2022/076221

§ 371 Date: Mar. 15, 2024

Related U.S. Application Data
(60) Provisional application No. 63/245,512, filed on Sep. 17, 2021.

(51) Int. Cl. C40B 30/04; C07K 16/00; C07K 16/10; G01N 33/569

(52) U.S. Cl. CPC C40B 30/04; C07K 16/005; C07K 16/1003; G01N 33/56983; C07K 2317/22; C07K 2317/24; C07K 2317/31; C07K 2317/35; C07K 2317/565; C07K 2317/567; C07K 2317/569; C07K 2317/76; C07K 2319/30; G01N 2333/165

(57) ABSTRACT

Methods for producing synthetic single-domain monoclonal antibody libraries using humanized llama nanobody framework sequences, libraries obtainable by the method, as well as antibodies selected from the libraries are described. In particular, synthetic single-domain monoclonal antibodies that specifically bind to the spike protein of SARS-COV-2 and neutralize SARS-COV-2 infection are described. Use of the disclosed antibodies for the detection, prophylaxis and treatment of SARS-COV-2 infection is described.

### CROSS REFERENCE TO RELATED APPLICATIONS

This application claims the benefit of U.S. Provisional Application No. 63/245,512, filed Sep. 17, 2021, which is herein incorporated by reference in its entirety.

### FIELD

This disclosure concerns a synthetic single-domain monoclonal antibody library having humanized llama nanobody framework regions and randomized complementarity determining regions (CDRs). This disclosure further concerns SARS-COV-2 neutralizing antibodies identified from the library.

### BACKGROUND

The severe acute respiratory syndrome-coronavirus 2 (SARS-COV-2) pandemic led to a worldwide emergency imposing massive strains on medical systems and a staggering number of deaths. The scientific community responded with unprecedented celerity to develop effective vaccines conferring protective immunity. Although vaccination reduces the number of hospitalizations, there is potential for the emergence of escape variants and ongoing viral transmission. Thus, there remains an ongoing need for cost-effective, high-throughput, adaptable pipelines capable of identifying effective therapeutics against SARS-COV-2 and other emerging pandemic threats.

SARS-COV-2 entry into host cells relies upon the envelope-anchored spike (S) glycoprotein. This large trimeric class I protein densely decorates the viral surface and recognizes host angiotensin-converting enzyme 2 (ACE2) and contains the fusion machinery needed for viral entry. During biogenesis, each S protomer in the mushroom shaped trimer is cleaved by cellular furin into S1 and S2 subunits responsible for target recognition and fusion, respectively. Each N-terminal S1 subunit contains a receptor binding domain (RBD) targeting ACE2 on the host cell. The RBD is connected to a hinge that enables its transition between an ‘up’ state capable of binding to ACE2 and a ‘down’ state in which the interaction with the receptor is hindered by the proximity of the adjacent RBD. Receptor binding in the ‘up’ position triggers a cascade of events including the TMPRSS2 mediated cleavage of the stalk forming S2 protomers to reveal hydrophobic fusion peptides (FP) at each N-terminal end of a long axial three helix bundle. Insertion of FPs into the membrane of the target cell is followed by a massive structural rearrangement resulting in its apposition with the viral envelope that leads to their fusion. Mutations in this targeting/fusion machine have been implicated in increased viral infectivity. As the driver of viral tropism, the RBD of the surface-exposed S protein is the focus of intense interest for the development of neutralizing antibodies and immunogens. Multiple variants of SARS-COV-2 have arisen independently in which N501, one of the key contact residues within the RBD, has been mutated to N501Y. This mutation increases binding affinity to human ACE2. These variants have multiple mutations in spike, which have been associated with increased transmission rates and reduced antibody neutralization. Structural insights into how antibodies bind to SARS-COV-2 variants are still needed, as are improved SARS-COV-2 neutralizing antibodies effective against circulating variants.

### SUMMARY

The present disclosure describes the generation of synthetic single-domain monoclonal antibody libraries derived from humanized llama nanobody framework sequences and randomized, diverse complementarity determining region (CDR) sequences. Also described are single-domain monoclonal antibodies selected from the libraries based on high affinity binding to SARS-COV-2 spike protein.

Provided herein are methods of making a synthetic single-domain monoclonal antibody library. In some implementations, the method includes introducing a diversity of nucleic acid molecules encoding complementarity determining region 1 (CDR1), CDR2 and CDR3 sequences between respective framework (FR) coding regions of a synthetic single-domain monoclonal antibody to generate nucleic acid molecules encoding a diversity of synthetic single-domain monoclonal antibodies with the same synthetic single-domain monoclonal antibody scaffold amino acid sequence. In some examples, the synthetic single-domain monoclonal antibody scaffold includes a FR1 sequence comprising SEQ ID NO: 1, a FR2 sequence comprising SEQ ID NO: 2, a FR3 sequence comprising SEQ ID NO: 3 and a FR4 sequence comprising SEQ ID NO: 4. Synthetic single-domain monoclonal antibody libraries obtainable by the disclosed method are further provided.

Also provided is a screening method for identifying a synthetic single-domain monoclonal antibody that binds to a target of interest. In some implementations, the method includes the use of a synthetic single-domain antibody library disclosed herein.

Further provided herein are single-domain monoclonal antibodies having the following formula: FR1-CDR1-FR2-CDR2-FR3-CDR3-FR4, wherein the amino acid sequence of FR1 comprises SEQ ID NO: 1; the amino acid sequence of FR2 comprises SEQ ID NO: 2; the amino acid sequence of FR3 comprises SEQ ID NO: 3; and/or the amino acid sequence of FR4 comprises SEQ ID NO: 4. Nucleic acid molecules and vectors encoding the single-domain monoclonal antibodies, as well as isolated host cells that include the nucleic acid molecules or vectors are further provided.

Also provided herein are single-domain monoclonal antibodies that specifically bind SARS-CoV-2 spike protein. In some implementations, the antibody includes the CDR sequences of antibody RBD-1-2G. In some examples, the antibody is capable of neutralizing SARS-COV-2.

Single-domain monoclonal antibodies conjugated to a detectable marker are further provided.

Also provided are fusion proteins that include a single-domain monoclonal antibody disclosed herein and a heterologous protein, such as an Fc protein, for example a human Fc protein. Further provided are bivalent antibodies and trivalent single-chain Fv antibodies that include a single-domain monoclonal antibody disclosed herein. Bispecific monoclonal antibodies that include a disclosed single-domain monoclonal antibody and a second antibody (or antigen-binding fragment) specific for a different antigen or epitope are also provided.

Further provided are nucleic acid molecules that encode a single-domain monoclonal antibody, fusion protein, bivalent antibody, trivalent single-chain Fv, or bispecific antibody disclosed herein. In some implementations, the nucleic acid molecule is operably linked to a promoter, such as a heterologous promoter. Also provided are vectors that include a disclosed nucleic acid molecule, and host cells that include such a vector.

Also provided are compositions that include a pharmaceutically acceptable carrier and a single-domain monoclonal antibody, fusion protein, bivalent antibody, trivalent single-chain Fv, bispecific antibody, nucleic acid molecule or vector disclosed herein.

Methods of producing a single-domain monoclonal antibody that specifically binds to a SARS-COV-2 spike protein are further provided. In some implementations, the method includes expressing a nucleic acid molecule encoding a disclosed single-domain monoclonal antibody in a host cell, and purifying the single-domain monoclonal antibody.

Also provided are methods of detecting the presence of a coronavirus in a biological sample from a subject. In some implementations, the method includes contacting the biological sample with an effective amount of a single-domain monoclonal antibody disclosed herein under conditions sufficient to form an immune complex; and detecting the presence of the immune complex in the biological sample. The presence of the immune complex in the biological sample indicates the presence of the coronavirus in the sample.

Further provided are methods of inhibiting a coronavirus infection in a subject. In some implementations, the method includes administering an effective amount of a single-domain monoclonal antibody, composition, fusion protein, bivalent antibody, trivalent single-chain Fv, bispecific antibody, nucleic acid molecule or vector disclosed herein to the subject.

The foregoing and other objects and features of the disclosure will become more apparent from the following detailed description, which proceeds with reference to the accompanying figures.

### BRIEF DESCRIPTION OF THE DRAWINGS

FIGS. 1A-1E: Discovery of anti-SARS-COV-2 Spike RBD nanobodies that block interactions with ACE2. (FIG. 1A) Parameters for construction of a synthetic humanized llama nanobody library. (FIG. 1B) Schematic of the selection strategy for identification of anti-RBD nanobodies using phage panning. (FIG. 1C) Octet binding profiles of RBD-1-1E, RBD-2-1F and RBD-1-2G against RBD-mFc (200 nM to 12.5 nM, 1:2 dilution). (FIG. 1D) Association (kₒₙ) and dissociation (k_{off}) rate constants and equilibrium dissociation constants (K_{D}) of nanobodies binding to RBD-mFc and S1-hFc. Global fit calculations for RBD-1-1E, RBD-2-1F, and RBD-1-2G used 200 nM to 12.5 nM; all others used 200 nM to 50 nM. (FIG. 1E) Nanobody-mediated inhibition of RBD-Fc binding to ACE2-Avi using AlphaLISA assay.

FIGS. 2A-2H: Multivalency improves affinity and inhibition of SARS-COV-2 infection in vitro. (FIGS. 2A-2C) Octet binding profiles for the (FIG. 2A) RBD-1-2G Nb, (FIG. 2B) RBD-1-2G-Fc and (FIG. 2C) RBD-1-2G-Trimer against RBD-His (100 nM to 6.25 nM, 1:2 dilution). (FIGS. 2D-2E) QD endocytosis assay using QD₆₀₈-RBD and ACE2-GFP HEK293T cells to visualize receptor binding. Shown is nanobody efficacy in reducing RBD internalization by (FIG. 2D) nanobody and (FIG. 2E) Fc constructs. N=duplicate wells, approximately 2500 cells and 1600 cells, respectively. (FIGS. 2F-2G) SARS-COV-2 pseudotyped particle entry assay using HEK293-ACE2 cells as target. Inhibition of pseudotyped particle entry was tested for nanobody (FIG. 2F) and Fc (FIG. 2G) constructs. Representative data from two independent experiments. Data represents mean inhibition per concentration (n=3), all error bars represent SEM. (FIG. 2H) Inhibition of SARS-COV-2 live virus infection with the RBD-1-2G and RBD-2-1F in various formats. Representative biological replicate with n=2. Technical replicates are n=2 per concentration, all error bars represent S.D.

FIGS. 3A-3C: Cryo-EM of SARS-COV-2 spike trimer and RBD binding nanobodies. (FIG. 3A) Cryo-EM analysis of nanobodies complexed with the RBD (up state) revealed two distinct binding modes. (FIG. 3B) Top and side views of the cryo-EM map of S-protein in complex with 3 molecules of RBD-1-2G. (FIG. 3C) Side view of the S-protein highlighting the spike monomer region refined during image processing is shown. The density containing the RBD in the laid state was used for the atomic model fitting refinement.

FIGS. 4A-4D: Examining the ability of RBD-1-2G to bind the UK variant (N501Y). (FIG. 4A) Maximum response values reached during the association phase by RBD-1-2G-Fc binding wildtype (WT) and United Kingdom (UK) variant RBDs. Differences in pH were achieved using PBS (7.4 pH) or 10 mM acetate buffers with 150 mM NaCl (pH 4-pH 6.0), all buffers contained 0.1% BSA and 0.02% Tween. (FIG. 4B) Maximum response values reached during the association phase by RBD-1-2G-Fc binding WT and UK variant S1 proteins. (FIGS. 4C-4D) SARS-COV-2 pseudotyped particle entry assay using HEK293-ACE2 cells as target. Inhibition of WT (FIG. 4C) and UK (FIG. 4D) pseudotyped particles treated with various RBD-1-2G and RBD-2-1F formats. Representative biological replicate with n=2. Technical replicates were n=3 per concentration, all error bars represent S.D.

FIGS. 5A-5E: Molecular dynamics of RBD-1-2G with wild type and B.1.1.7 RBD variant. (FIGS. 5A-5B) Sausage plot representation of RBD-1-2G in complex with WT RBD (FIG. 5A) and B.1.1.7 RBD (FIG. 5B). Regions showing the most flexibility are indicated (470-490 and 355-375). (FIG. 5C) Energy contribution of RBD-1-2G residues in complex with WT RBD and B.1.1.7 (N501Y) RBD for the total free energy of binding. Highest contribution is observed for residues R76 (RBD-1-2G) and E484 (RBD). (FIGS. 5D-5E) Atomic model obtained after the atomic model fitting and molecular dynamic (MD) simulations of the (FIG. 5D) WT RBD and (FIG. 5E) B.1.1.7 RBD in complex with RBD-1-2G, highlighting the difference of the interactions involving E484 (RBD).

FIGS. 6A-6D: Nanobody purification and quality control. (FIG. 6A) Representative IMAC purification. T-total protein (lysate), L-column load (clarified lysate), FT-column flow through, W-column wash. (FIG. 6B) Representative preparative SDS-PAGE after size exclusion column purification (SEC). L-column load. (FIG. 6C) SDS-PAGE Coomassie Blue staining of purified nanobodies. All gels in FIGS. 6A-6C are SDS-PAGE/Coomassie staining with mass of protein standards noted in kDa. Solid bars indicate fractions pooled. (FIG. 6D) Electrospray ionization mass spectrometry (ESI-MS) of a representative purified nanobody.

FIGS. 7A-7H: Octet binding profiles for immobilized nanobodies binding RBD-mFC at concentrations of 200 nM, 100 nM and 50 nM.

FIGS. 8A-8K: Octet binding profiles for immobilized nanobodies binding S1-hFc at concentrations of 200 nM, 100 nM and 50 nM. RBD-1-2G (FIG. 8C), RBD-2-1F (FIG. 8B) and RBD-1-1E (FIG. 8A) also included concentrations of 25 nM and 12.5 nM.

FIGS. 9A-9C: QD ACE2-GFP endocytosis assay with nanobody treatment. (FIG. 9A) Representative images for nanobody inhibition of QD endocytosis. Representative image montage of ACE2-GFP HEK293T cells treated with QD₆₀₈-RBD that was preincubated for 30 minutes with RBD-2-1F, RBD-1-2G, RBD-1-1E, or RBD-2-1E starting at a concentration of 10 uM. Cells were treated for a total of three hours. Digital phase contrast (DPC) was used to visualize cell bodies. Scale bar, 20 μm. (FIGS. 9B-9C) Quantification of (FIG. 9B) QD₆₀₈-RBD and (FIG. 9C) ACE2-GFP using high-content image analysis in each channel. Data was normalized to Optimem I treated cells (100%) and QD608-RBD alone (0%). N=approximately 2500 cells from duplicate wells, representative of three independent experiments. Curves fit using non-linear regression. Error bars indicate S.D.

FIGS. 10A-10C: QD ACE2-GFP endocytosis assay with Fc treatment. (FIG. 10A) Representative images for Fc inhibition of QD endocytosis. Representative image montage of ACE2-GFP HEK293T cells treated with QD₆₀₈-RBD that was preincubated for 30 minutes with RBD-2-1F-Fc, RBD-1-2G-Fc, RBD-1-1E-Fc, or RBD-2-1E-Fc starting at a concentration of 5 μM or 10 μM. Cells were treated for a total of three hours. Digital phase contrast (DPC) was used to visualize cell bodies. Scale bar, 20 μm. (FIGS. 10B-10C) Quantification of (FIG. 10B) QD₆₀₈-RBD and (FIG. 10C) ACE2-GFP using high-content image analysis in each channel. Data was normalized to Optimem I treated cells (100%) and QD₆₀₈-RBD alone (0%). N=approximately 1600 cells from duplicate wells, representative of three independent experiments. Curves fit using non-linear regression. Error bars indicate S.D.

FIGS. 11A-11E: Global Fit curves of 1-2G-Fc and 2-1F-Fc binding to RBD-mFc. (FIGS. 11A-11B) Octet binding profiles using either RBD-1-2G-Fc (FIG. 11A) or RBD-2-1F-Fc (FIG. 11B) as load protein. Association of RBD-mFc ranging from 200 nM to 6.25 nM (1:2 serial dilutions) were used for global curve fitting. (FIGS. 11C-11D) Octet biosensors were loaded with RBD-mFc, then exposed to various concentrations of RBD-1-2G-Fc (FIG. 11C) or RBD-2-1F-Fc (FIG. 11D) (200 nM to 6.25 nM, 1:2 serial dilutions). (FIG. 11E) Calculations from global fit modeling for FIGS. 11A-11D.

FIGS. 12A-12B: SARS-COV-2 pseudotyped particle assay for non-blockers. SARS-COV-2 pseudotyped particle entry assay for nanobody (FIG. 12A) and Fc (FIG. 12B) formats.

FIGS. 13A-13B: Atomic fit models of the RBD/ACE2/nanobody interactions. (FIG. 13A) Model of RBD-1-2G binding overlaps with the ACE2 binding site, while RBD-1-1G fails to inhibit binding. (FIG. 13B) Overlap of RBD-2-1F and RBD-1-2G suggesting similar epitopes are targeted by ‘Group 1’ binders.

FIG. 14: RBD region sequence overlaps.

FIG. 15: CryoEM workflow chart and refinement strategy.

FIGS. 16A-16B: The effect of lyophilization on RBD-1-2G nanobody. RBD-1-2G vs a lyophilized reconstituted sample were tested for their ability to inhibit live virus infection. The top concentrations were 15.2 uM for the untreated RBD-1-2G and 15.8 UM for the reconstituted lyophilized RBD-1-2G sample, then diluted using 1:2 dilutions in dPBS. Technical replicates were n=3 per concentration, all error bars represent S.D. (FIG. 16B) Bar graph of percent CPE rescue from the live virus assay.

FIGS. 17A-17B: Root mean square deviation (RMSD) of the MD triplicates per column for RBD-1-2G in complex with the (FIG. 17A) WT RBD and (FIG. 17B) B.1.1.7 RBD showed that the systems were stable.

FIGS. 18A-18D: Root mean square fluctuation (RMSF) for the (FIG. 18A) WT RBD residues and (FIG. 18B) B.1.1.7 RBD residues, showing the same pattern of variations. Both graphs show two main peaks corresponding to residues 355-375 and 470-490. (FIGS. 18C-18D) The fluctuation of the residues of the RBD-1-2G in complex with (FIG. 18C) WT RBD and (FIG. 18D) B.1.1.7 RBD.

FIGS. 19A-19B: Hydrogen bonds and salt bridge interactions per time of the MD simulations in triplicate for the systems (per column) containing RBD-1-2G with (FIG. 19A) WT RBD and (FIG. 19B) with B.1.1.7 RBD.

FIGS. 20A-20B: Human membrane proteome array (MPA) results identified during a cross-reactivity screen (FIG. 20A). Follow-up validation screen to determine the reactivity of MIEF1 protein to RBD-1-2G (FIG. 20B).

FIG. 21: Heatmap of residues in RBD-1-2G vs RBD in WT and B.1.1.7 variant.

### SEQUENCE LISTING

The Sequence Listing is submitted as an ST.26 Sequence Listing XML file, named 4239-107021-02.xml, created on Sep. 1, 2022, having a size of 8,469 bytes, which is incorporated by reference herein. In the accompanying sequence listing:
SEQ ID NO: 1 is the amino acid sequence of Framework 1 (FR1):

EVQLVESGGGLVQPGGSLRLSCAAS

SEQ ID NO: 2 is the amino acid sequence of Framework 2 (FR2):

MGWFRQAPGKGRELVAA

SEQ ID NO: 3 is the amino acid sequence of Framework 3 (FR3):

YPDSVEGRFTISRDNAKRMVYLQMNSLRAEDTAVYYCA

SEQ ID NO: 4 is the amino acid sequence of Framework 4 (FR4):

WGQGTQVTVSS

SEQ ID NO: 5 is the amino acid sequence of single-domain antibody RBD-1-2G:

EVQLVESGGGLVQPGGSLRLSCAASGFSSIVYM
GWFRQAPGKGRELVAAIDASGSTTNYPDSVEGR
FTISRDNAKRMVYLQMNSLRAEDTAVYYCAIAY
FTSPEYVVSQGWGQGTQVTVSS
(CDR1 = residues 26-32;
CDR2 = residues 50-58;
CDR3 = residues 97-110).

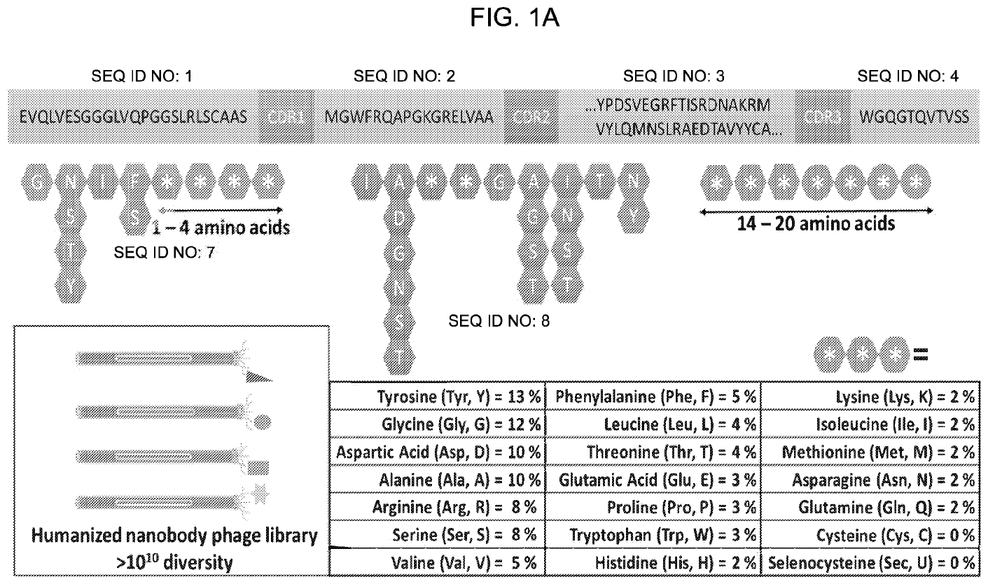

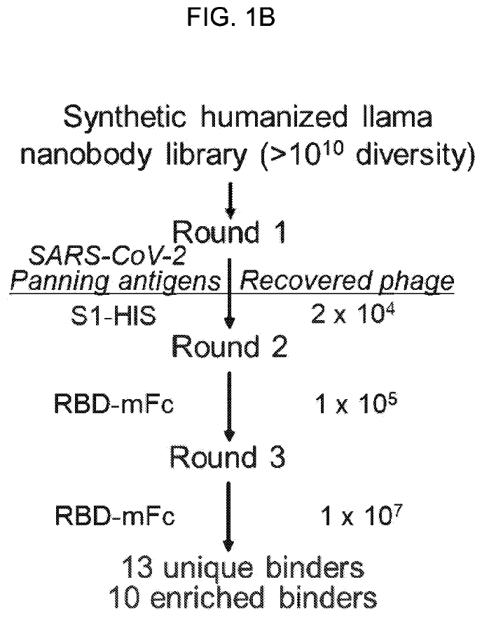

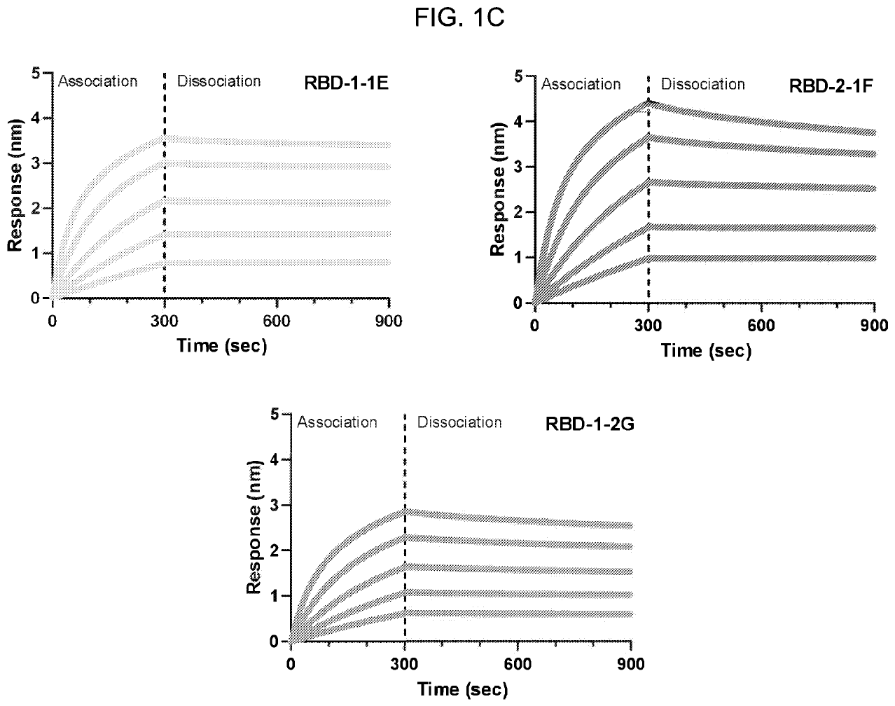

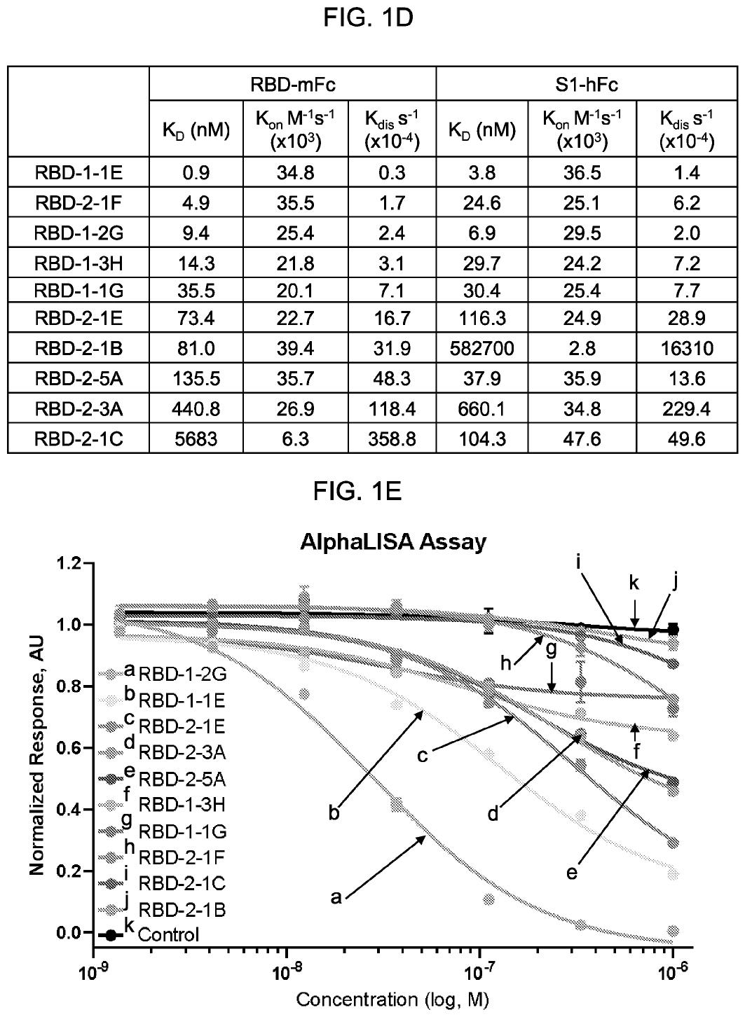

SEQ ID NO: 6 is the amino acid sequence of a protein tag:
   GQAGQHHHHHHGAYPYDVPDYAS, wherein residues 1-5, 12-13 and 23 are spacer residues, residues 6-11 are a His tag, and residues 14-22 are a human influenza hemagglutinin (HA) tag.
SEQ ID NO: 7 is an amino acid consensus sequence of a randomized CDR1: GX₁IX₂X₃, wherein X₁ is N, S, T or Y; X₂ is F or S; and X₃ is 1 to 4 amino acid residues individually selected from Y, G, D, A, R, S, V, F, L, T, E, P, W, H, K, I, M, N and Q (see FIG. 1A).
SEQ ID NO: 8 is an amino acid consensus sequence of a randomized CDR2:
   IX₁X₂X₂GX₃X₄TX₅, wherein X₁ is A, D, G, N, S or T; X₂ is any amino acid selected from Y, G, D, A, R, S, V, F, L, T, E, P, W, H, K, I, M, N and Q; X₃ is A, G, S or T; X₄ is I, N, S or T; and X₅ is N or Y (see FIG. 1A).
SEQ ID NO: 9 is the amino acid sequence of a peptide linker (GGGGSGGGGSGGGGS).
SEQ ID NO: 10 is the amino acid sequence of a mutated furin site (GSAS).

### DETAILED DESCRIPTION

### I. Abbreviations

ACE2 angiotensin converting enzyme 2
CDR complementarity determining region
CFU colony forming unit
CoV coronavirus
CPE cytopathic effect
CV column volume
EB equilibration buffer
FP fusion peptide
GFP green fluorescent protein
IMAC immobilized-metal affinity chromatography
MD molecular dynamic
MLV murine leukemia virus
MPA membrane proteome array
Nb nanobody
PP pseudotyped particle
QD quantum dot
RBD receptor binding domain
RBM receptor binding motif
RMSD root mean square deviation
RMSF root mean square fluctuation
RT room temperature
S spike
SARS severe acute respiratory syndrome
SEC size exclusion chromatography
UK United Kingdom
WT wild type

### II. Summary of Terms

Unless otherwise noted, technical terms are used according to conventional usage. Definitions of many common terms in molecular biology may be found in Krebs et al. (eds.), Lewin's genes XII, published by Jones & Bartlett Learning, 2017. As used herein, the singular forms “a,” “an,” and “the,” refer to both the singular as well as plural, unless the context clearly indicates otherwise. For example, the term “an antigen” includes singular or plural antigens and can be considered equivalent to the phrase “at least one antigen.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting.

To facilitate review of the various implementations, the following explanations of terms are provided:

About: Unless context indicated otherwise, “about” refers to plus or minus 5% of a reference value. For example, “about” 100 refers to 95 to 105.

Administration: The introduction of an agent, such as a disclosed antibody, into a subject by a chosen route. Administration can be local or systemic. For example, if the chosen route is intravascular, the agent (such as an antibody) is administered by introducing the composition into a blood vessel of the subject. Exemplary routes of administration include, but are not limited to, oral, injection (such as subcutaneous, intramuscular, intradermal, intraperitoneal, and intravenous), sublingual, rectal, transdermal (for example, topical), intranasal, vaginal, and inhalation routes.

Amino acid substitution: The replacement of one amino acid in a polypeptide with a different amino acid.

Antibody: An immunoglobulin, antigen-binding fragment, or derivative thereof, that specifically binds and recognizes an analyte (antigen) such as a coronavirus spike protein, such as a spike protein from SARS-COV-2. The term “antibody” is used herein in the broadest sense and encompasses various antibody structures, including but not limited to monoclonal antibodies, polyclonal antibodies, multispecific antibodies (e.g., bispecific antibodies), single-domain antibodies, and antigen binding fragments, so long as they exhibit the desired antigen-binding activity.

Non-limiting examples of antibodies include, for example, intact immunoglobulins and variants and fragments thereof that retain binding affinity for the antigen. Examples of antigen binding fragments include but are not limited to Fv, Fab, Fab′, Fab′-SH, F(ab′)₂; diabodies; linear antibodies; single-chain antibody molecules (e.g. scFv); and multispecific antibodies formed from antibody fragments. Antibody fragments include antigen binding fragments either produced by the modification of whole antibodies or those synthesized de novo using recombinant DNA methodologies (see, e.g., Kontermann and Dübel (Eds.), Antibody Engineering, Vols. 1-2, 2^{nd} ed., Springer-Verlag, 2010).

Antibodies also include genetically engineered forms such as chimeric antibodies (such as humanized murine antibodies) and heteroconjugate antibodies (such as bispecific antibodies).

An antibody may have one or more binding sites. If there is more than one binding site, the binding sites may be identical to one another or may be different. For instance, a naturally-occurring immunoglobulin has two identical binding sites, a single-chain antibody or Fab fragment has one binding site, while a bispecific or bifunctional antibody has two different binding sites.

Typically, a naturally occurring immunoglobulin has heavy (H) chains and light (L) chains interconnected by disulfide bonds. Mammalian immunoglobulin genes include the kappa, lambda, alpha, gamma, delta, epsilon and mu constant region genes, as well as the myriad immunoglobulin variable domain genes. There are two types of light chain, lambda (λ) and kappa (κ). There are five main heavy chain classes (or isotypes) which determine the functional activity of a mammalian antibody molecule: IgM, IgD, IgG, IgA and IgE. Antibody isotypes not found in mammals include IgX, IgY, IgW and IgNAR. IgY is the primary antibody produced by birds and reptiles, and has some functionally similar to mammalian IgG and IgE. IgW and IgNAR antibodies are produced by cartilaginous fish, while IgX antibodies are found in amphibians.

Each heavy and light chain contains a constant region (or constant domain) and a variable region (or variable domain). In combination, the heavy and the light chain variable regions specifically bind the antigen.

References to “VH” or “VH” refer to the variable region of an antibody heavy chain, including that of an antigen binding fragment, such as Fv, scFv, dsFv or Fab. References to “VL” or “VL” refer to the variable domain of an antibody light chain, including that of an Fv, scFv, dsFv or Fab.

The VH and VL contain a “framework” region interrupted by three hypervariable regions, also called “complementarity-determining regions” or “CDRs” (see, e.g., Kabat et al., Sequences of Proteins of Immunological Interest, 5^{th} ed., NIH Publication No. 91-3242, Public Health Service, National Institutes of Health, U.S. Department of Health and Human Services, 1991). The sequences of the framework regions of different light or heavy chains are relatively conserved within a species. The framework region of an antibody, that is the combined framework regions of the constituent light and heavy chains, serves to position and align the CDRs in three-dimensional space.

The CDRs are primarily responsible for binding to an epitope of an antigen. The amino acid sequence boundaries of a given CDR can be readily determined using any of a number of well-known schemes, including those described by Kabat et al. (Sequences of Proteins of Immunological Interest, 5^{th} ed., NIH Publication No. 91-3242, Public Health Service, National Institutes of Health, U.S. Department of Health and Human Services, 1991; “Kabat” numbering scheme), Al-Lazikani et al., (“Standard conformations for the canonical structures of immunoglobulins,” J. Mol. Bio., 273 (4): 927-948, 1997; “Chothia” numbering scheme), and Lefranc et al. (“IMGT unique numbering for immunoglobulin and T cell receptor variable domains and Ig superfamily V-like domains,” Dev. Comp. Immunol., 27 (1): 55-77, 2003; “IMGT” numbering scheme). The CDRs of each chain are typically referred to as CDR1, CDR2, and CDR3 (from the N-terminus to C-terminus), and are also typically identified by the chain in which the particular CDR is located. Thus, a V_{H} CDR3 is the CDR3 from the VH of the antibody in which it is found, whereas a VL CDR1 is the CDR1 from the V_{L} of the antibody in which it is found. Light chain CDRs are sometimes referred to as LCDR1, LCDR2, and LCDR3. Heavy chain CDRs are sometimes referred to as HCDR1, HCDR2, and HCDR3.

In some implementations, a disclosed antibody includes a heterologous constant domain. For example, the antibody includes a constant domain that is different from a native constant domain, such as a constant domain including one or more modifications (such as the “LS” mutation) to increase half-life.

A “monoclonal antibody” is an antibody obtained from a population of substantially homogeneous antibodies, that is, the individual antibodies comprising the population are identical and/or bind the same epitope, except for possible variant antibodies, for example, containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein. In some examples, single-domain antibodies are isolated from a phage display library. Monoclonal antibodies can have conservative amino acid substitutions which have substantially no effect on antigen binding or other immunoglobulin functions. (See, for example, Greenfield (Ed.), Antibodies: A Laboratory Manual, 2^{nd} ed. New York: Cold Spring Harbor Laboratory Press, 2014).

A “single-domain antibody” refers to an antibody having a single domain (a variable domain) that is capable of specifically binding an antigen, or an epitope of an antigen, in the absence of an additional antibody domain. Single-domain antibodies include, for example, camelid V_{H}H antibodies, V_{NAR} antibodies, VH domain antibodies and VL domain antibodies. V_{NAR} antibodies are produced by cartilaginous fish, such as nurse sharks, wobbegong sharks, spiny dogfish and bamboo sharks. Camelid V_{H}H antibodies are produced by several species including camel, llama, alpaca, dromedary, and guanaco, which produce heavy chain antibodies that are naturally devoid of light chains.

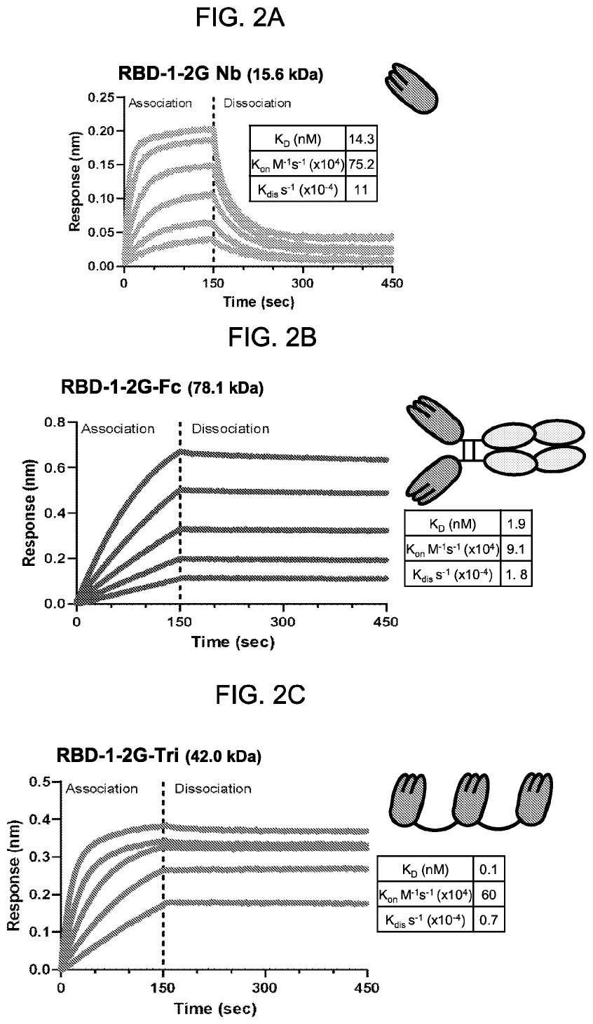

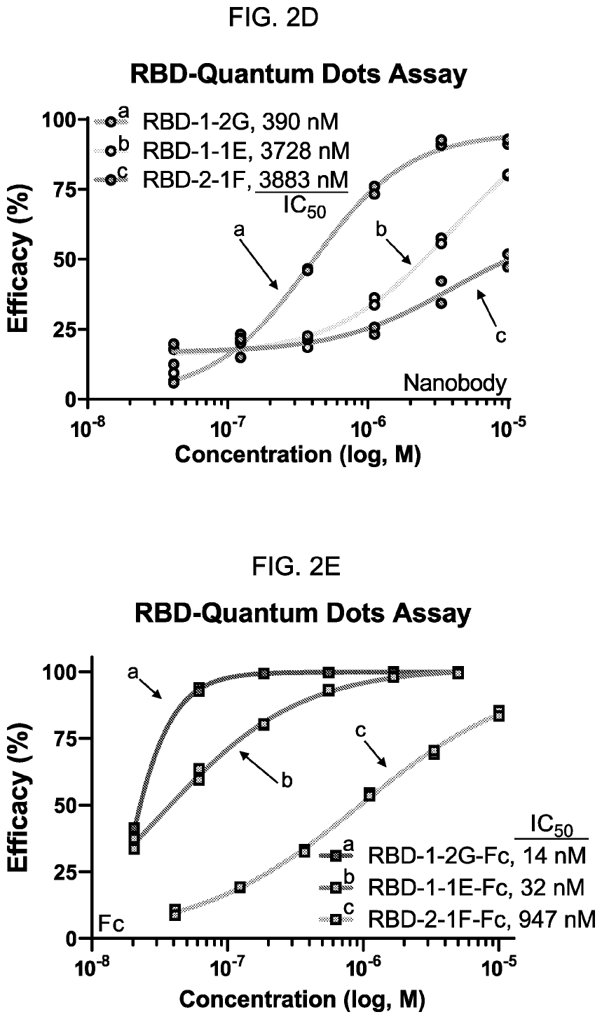

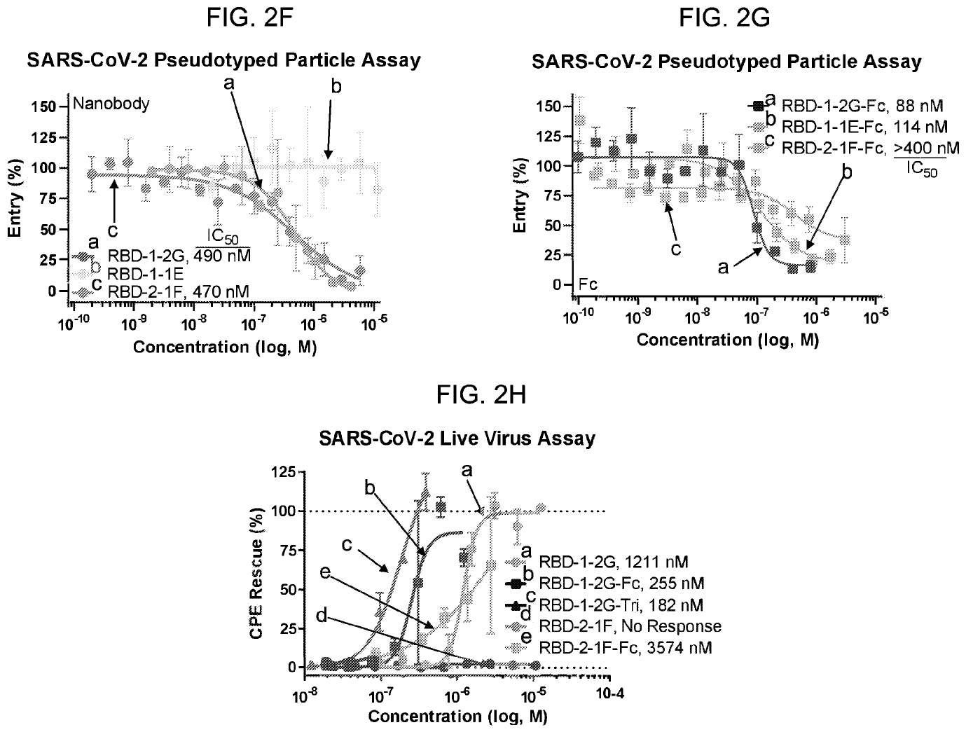

In the context of the present disclosure, a “bivalent antibody” is a molecule comprising two single-domain monoclonal antibody (“nanobody”) monomers. In some implementations, the monomers are fused to an Ig Fc region (see schematic in FIG. 2B).

In the context of the present disclosure, a “trivalent single-chain Fv” is a molecule comprising three single-domain antibody (“nanobody”) monomers. In some implementations, the monomers are connected by flexible peptide linkers (see schematic in FIG. 2C).

A “humanized” antibody or antigen binding fragment includes a human framework region and one or more CDRs from a non-human (such as a mouse, rat, or synthetic) antibody or antigen binding fragment. The non-human antibody or antigen binding fragment providing the CDRs is termed a “donor,” and the human antibody or antigen binding fragment providing the framework is termed an “acceptor.” In one implementation, all the CDRs are from the donor immunoglobulin in a humanized immunoglobulin. Constant regions need not be present, but if they are, they can be substantially identical to human immunoglobulin constant regions, such as at least about 85-90%, such as about 95% or more identical. Hence, all parts of a humanized antibody or antigen binding fragment, except possibly the CDRs, are substantially identical to corresponding parts of natural human antibody sequences.

A “chimeric antibody” is an antibody which includes sequences derived from two different antibodies, which typically are of different species. In some examples, a chimeric antibody includes one or more CDRs and/or framework regions from one human antibody and CDRs and/or framework regions from another human antibody.

A “fully human antibody” or “human antibody” is an antibody which includes sequences from (or derived from) the human genome, and does not include sequence from another species. In some implementations, a human antibody includes CDRs, framework regions, and (if present) an Fc region from (or derived from) the human genome. Human antibodies can be identified and isolated using technologies for creating antibodies based on sequences derived from the human genome, for example by phage display or using transgenic animals (see, e.g., Barbas et al. Phage display: A Laboratory Manuel. 1^{st} Ed. New York: Cold Spring Harbor Laboratory Press, 2004. Print.; Lonberg, Nat. Biotech., 23:1117-1125, 2005; Lonenberg, Curr. Opin. Immunol., 20:450-459, 2008).

Antibody that neutralizes SARS-COV-2: An antibody, such as a single-domain monoclonal antibody, that specifically binds to a SARS-COV-2 antigen (such as the spike protein) in such a way as to inhibit a biological function associated with SARS-COV-2 that inhibits infection. The antibody can neutralize the activity of SARS-COV-2. For example, an antibody that neutralizes SARS-COV-2 may interfere with the virus by binding it directly and limiting entry into cells. Alternately, an antibody may interfere with one or more post-attachment interactions of the pathogen with a receptor, for example, by interfering with viral entry using the receptor. In some examples, an antibody that is specific for a coronavirus spike protein neutralizes the infectious titer of SARS-COV-2.

In some implementations, an antibody that specifically binds to SARS-COV-2 and neutralizes SARS-COV-2 inhibits infection of cells, for example, by at least 50% compared to a control antibody or antigen binding fragment.

A “broadly neutralizing antibody” is an antibody that binds to and inhibits the function of related antigens, such as antigens that share at least 85%, 90%, 95%, 96%, 97%, 98% or 99% identity antigenic surface of antigen. With regard to an antigen from a pathogen, such as a virus, the antibody can bind to and inhibit the function of an antigen from more than one class and/or subclass of the pathogen. For example, with regard to a coronavirus, the antibody can bind to and inhibit the function of an antigen, such as the spike protein from coronaviruses including SARS-CoV-2.

Antigen: A compound, composition, or substance that can stimulate the production of antibodies or a T-cell response in an animal, including compositions that are injected or absorbed into an animal. An antigen reacts with the products of specific humoral or cellular immunity, including those induced by heterologous immunogens. In some implementations herein, the antigen is a viral antigen, such as a SARS-COV-2 spike protein.

Binding affinity: Affinity of an antibody for an antigen. In one embodiment, affinity is calculated by a modification of the Scatchard method described by Frankel et al., Mol. Immunol., 16:101-106, 1979. In another embodiment, binding affinity is measured by an antigen/antibody dissociation rate. In another embodiment, a high binding affinity is measured by a competition radioimmunoassay. In another embodiment, binding affinity is measured by ELISA. In some embodiments, binding affinity is measured using the Octet system (Creative Biolabs), which is based on bio-layer interferometry (BLI) technology. In other embodiments, Kd is measured using surface plasmon resonance assays using a BIACORES-2000 or a BIACORES-3000 (BIAcore, Inc., Piscataway, N.J.). In other embodiments, antibody affinity is measured by flow cytometry or by surface plasmon reference. An antibody that “specifically binds” an antigen (such as a coronavirus spike protein) is an antibody that binds the antigen with high affinity and does not significantly bind other unrelated antigens.

Biological sample: A sample obtained from a subject. Biological samples include all clinical samples useful for detection of disease or infection in subjects, including, but not limited to, cells, tissues, and bodily fluids, such as blood, derivatives and fractions of blood (such as serum), cerebrospinal fluid, as well as biopsied or surgically removed tissue, for example tissues that are unfixed, frozen, or fixed in formalin or paraffin. In a particular example, a biological sample is obtained from a subject having or suspected of having a SARS-COV-2 infection.

Bispecific antibody: A recombinant molecule composed of two different antigen binding domains that consequently binds to two different antigenic epitopes. Bispecific antibodies include chemically or genetically linked molecules of two antigen-binding domains. The antigen binding domains can be linked using a linker. The antigen binding domains can be monoclonal antibodies, antigen-binding fragments (e.g., Fab, scFv), or combinations thereof. A bispecific antibody can include one or more constant domains, but does not necessarily include a constant domain.

Conditions sufficient to form an immune complex: Conditions which allow an antibody to bind to its cognate epitope to a detectably greater degree than, and/or to the substantial exclusion of, binding to substantially all other epitopes. Conditions sufficient to form an immune complex are dependent upon the format of the binding reaction and typically are those utilized in immunoassay protocols or those conditions encountered in vivo. See Greenfield (Ed.), Antibodies: A Laboratory Manual, 2^{nd} ed. New York: Cold Spring Harbor Laboratory Press, 2014, for a description of immunoassay formats and conditions. The conditions employed in the methods are “physiological conditions” which include reference to conditions (e.g., temperature, osmolarity, pH) that are typical inside a living mammal or a mammalian cell. While it is recognized that some organs are subject to extreme conditions, the intra-organismal and intracellular environment normally lies around pH 7 (e.g., from pH 6.0 to pH 8.0, more typically pH 6.5 to 7.5), contains water as the predominant solvent, and exists at a temperature above 0° C. and below 50° C. Osmolarity is within the range that is supportive of cell viability and proliferation.

The formation of an immune complex can be detected through conventional methods, for instance immunohistochemistry (IHC), immunoprecipitation (IP), flow cytometry, immunofluorescence microscopy, ELISA, immunoblotting (for example, Western blot), magnetic resonance imaging (MRI), computed tomography (CT) scans, radiography, and affinity chromatography.

Conjugate: A complex of two molecules linked together, for example, linked together by a covalent bond. In one embodiment, an antibody is linked to an effector molecule; for example, an antibody that specifically binds to SARS-COV-2 covalently linked to an effector molecule, such as a detectable label. The linkage can be by chemical or recombinant means. In one embodiment, the linkage is chemical, wherein a reaction between the antibody moiety and the effector molecule has produced a covalent bond formed between the two molecules to form one molecule. A peptide linker (short peptide sequence) can optionally be included between the antibody and the effector molecule. Because conjugates can be prepared from two molecules with separate functionalities, such as an antibody and an effector molecule, they are also sometimes referred to as “chimeric molecules.”

Conservative variants: “Conservative” amino acid substitutions are those substitutions that do not substantially affect or decrease a function of a protein, such as the ability of the protein to interact with a target protein. For example, a SARS-COV-2-specific antibody can include up to 1, 2, 3, 4, 5, 6, 7, 8, 9, or up to 10 conservative substitutions compared to a reference antibody sequence and retain specific binding activity for spike protein binding, and/or SARS-COV-2 neutralization activity. The term conservative variation also includes the use of a substituted amino acid in place of an unsubstituted parent amino acid.

Individual substitutions, deletions or additions which alter, add or delete a single amino acid or a small percentage of amino acids (for instance less than 5%, in some embodiments less than 1%) in an encoded sequence are conservative variations where the alterations result in the substitution of an amino acid with a chemically similar amino acid.

The following six groups are examples of amino acids that are considered to be conservative substitutions for one another:
1) Alanine (A), Serine(S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

Non-conservative substitutions are those that reduce an activity or function of the antibody, such as the ability to specifically bind to a coronavirus spike protein. For instance, if an amino acid residue is essential for a function of the protein, even an otherwise conservative substitution may disrupt that activity. Thus, a conservative substitution does not alter the basic function of a protein of interest.

Contacting: Placement in direct physical association; includes both in solid and liquid form, which can take place either in vivo or in vitro. Contacting includes contact between one molecule and another molecule, for example the amino acid on the surface of one polypeptide, such as an antigen, that contacts another polypeptide, such as an antibody. Contacting can also include contacting a cell for example by placing an antibody in direct physical association with a cell.

Control: A reference standard. In some embodiments, the control is a negative control, such as sample obtained from a healthy patient not infected with a coronavirus. In other embodiments, the control is a positive control, such as a tissue sample obtained from a patient diagnosed with a coronavirus infection. In still other embodiments, the control is a historical control or standard reference value or range of values (such as a previously tested control sample, such as a group of patients with known prognosis or outcome, or group of samples that represent baseline or normal values).

A difference between a test sample and a control can be an increase or conversely a decrease. The difference can be a qualitative difference or a quantitative difference, for example a statistically significant difference. In some examples, a difference is an increase or decrease, relative to a control, of at least about 5%, such as at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, or at least about 500%.

Coronavirus: A family of positive-sense, single-stranded RNA viruses that are known to cause severe respiratory illness. Viruses from the coronavirus family that are currently known to infect humans from the alphacoronavirus and betacoronavirus genera. Additionally, it is believed that the gammacoronavirus and deltacoronavirus genera may infect humans in the future.

Non-limiting examples of betacoronaviruses include SARS-COV-2, Middle East respiratory syndrome coronavirus (MERS-COV), severe acute respiratory syndrome coronavirus (SARS-COV), human coronavirus HKU1 (HKU1-CoV), human coronavirus OC43 (OC43-CoV), murine hepatitis virus (MHV-CoV), bat SARS-like coronavirus WIV1 (WIV1-CoV), and human coronavirus HKU9 (HKU9-CoV). Non-limiting examples of alphacoronaviruses include human coronavirus 229E (229E-CoV), human coronavirus NL63 (NL63-CoV), porcine epidemic diarrhea virus (PEDV), and transmissible gastroenteritis coronavirus (TGEV). A non-limiting example of a deltacoronaviruses is the swine delta coronavirus (SDCV).

The viral genome is capped, polyadenylated, and covered with nucleocapsid proteins. The coronavirus virion includes a viral envelope containing type I fusion glycoproteins referred to as the spike(S) protein. Most coronaviruses have a common genome organization with the replicase gene.

COVID-19: The disease caused by the coronavirus SARS-COV-2.

Degenerate variant: In the context of the present disclosure, a “degenerate variant” refers to a polynucleotide encoding a polypeptide (such as an antibody heavy or light chain) that includes a sequence that is degenerate as a result of the genetic code. There are 20 natural amino acids, most of which are specified by more than one codon. Therefore, all degenerate nucleotide sequences encoding a peptide are included as long as the amino acid sequence of the peptide encoded by the nucleotide sequence is unchanged.

Detectable label: A detectable molecule (also known as a detectable marker) that is conjugated directly or indirectly to a second molecule, such as an antibody, to facilitate detection of the second molecule. For example, the detectable label can be capable of detection by ELISA, spectrophotometry, flow cytometry, microscopy or diagnostic imaging techniques (such as CT scans, MRIs, ultrasound, fiberoptic examination, and laparoscopic examination). Specific, non-limiting examples of detectable markers include fluorophores, chemiluminescent agents, enzymatic linkages, radioactive isotopes and heavy metals or compounds (for example super paramagnetic iron oxide nanocrystals for detection by MRI). Methods for using detectable markers and guidance in the choice of detectable markers appropriate for various purposes are discussed for example in Green and Sambrook (Molecular Cloning: A Laboratory Manual, 4^{th} ed., New York: Cold Spring Harbor Laboratory Press, 2012) and Ausubel et al. (Eds.) (Current Protocols in Molecular Biology, New York: John Wiley and Sons, including supplements, 2017).

Detecting: To identify the existence, presence, or fact of something.

Effective amount: A quantity of a specific substance sufficient to achieve a desired effect in a subject to whom the substance is administered. For instance, this can be the amount necessary to inhibit a coronavirus infection, such as a SARS-COV-2 infection, or to measurably alter outward symptoms of such an infection.

In one example, a desired response is to inhibit or reduce or prevent SARS-COV-2 infection. The SARS-COV-2 infection does not need to be completely eliminated or reduced or prevented for the method to be effective.

In some embodiments, administration of an effective amount of a disclosed antibody (or composition or conjugate thereof) that binds to a coronavirus spike protein can reduce or inhibit a SAR-COV-2 infection (for example, as measured by infection of cells, or by number or percentage of subjects infected by the coronavirus or by an increase in the survival time of infected subjects, or reduction in symptoms associated with the infection) by a desired amount, for example by at least 10%, at least 20%, at least 50%, at least 60%, at least 70%, at least 80%, at least 90%, at least 95%, at least 98%, or even at least 100% (elimination or prevention of detectable infection), as compared to a suitable control.

The effective amount of an antibody that specifically binds the coronavirus spike protein that is administered to a subject to inhibit infection will vary depending upon a number of factors associated with that subject, for example the overall health and/or weight of the subject. An effective amount can be determined by varying the dosage and measuring the resulting response, such as, for example, a reduction in pathogen titer. Effective amounts also can be determined through various in vitro, in vivo or in situ immunoassays.

An effective amount encompasses a fractional dose that contributes in combination with previous or subsequent administrations to attaining an effective response. For example, an effective amount of an agent can be administered in a single dose, or in several doses, for example daily, during a course of treatment lasting several days or weeks. However, the effective amount can depend on the subject being treated, the severity and type of the condition being treated, and the manner of administration. A unit dosage form of the agent can be packaged in an amount, or in multiples of the effective amount, for example, in a vial (e.g., with a pierceable lid) or syringe having sterile components.

Effector molecule: A molecule intended to have or produce a desired effect; for example, a desired effect on a cell to which the effector molecule is targeted, or a detectable marker. Effector molecules can include, for example, polypeptides and small molecules. Some effector molecules may have or produce more than one desired effect.

Epitope: An antigenic determinant. These are particular chemical groups or peptide sequences on a molecule that are antigenic, such that they elicit a specific immune response, for example, an epitope is the region of an antigen to which B and/or T cells respond. An antibody can bind to a particular antigenic epitope, such as an epitope on a coronavirus spike protein.

Expression: Transcription or translation of a nucleic acid sequence. For example, an encoding nucleic acid sequence (such as a gene) can be expressed when its DNA is transcribed into RNA or an RNA fragment, which in some examples is processed to become mRNA. An encoding nucleic acid sequence (such as a gene) may also be expressed when its mRNA is translated into an amino acid sequence, such as a protein or a protein fragment. In a particular example, a heterologous gene is expressed when it is transcribed into an RNA. In another example, a heterologous gene is expressed when its RNA is translated into an amino acid sequence. Regulation of expression can include controls on transcription, translation, RNA transport and processing, degradation of intermediary molecules such as mRNA, or through activation, inactivation, compartmentalization or degradation of specific protein molecules after they are produced.

Expression control sequences: Nucleic acid sequences that regulate the expression of a heterologous nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcriptional terminators, a start codon (ATG) in front of a protein-encoding gene, splice signals for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

Expression vector: A vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Non-limiting examples of expression vectors include cosmids, plasmids (e.g., naked or contained in liposomes) and viruses (e.g., lentiviruses, retroviruses, adenoviruses, and adeno-associated viruses) that incorporate the recombinant polynucleotide.

A polynucleotide can be inserted into an expression vector that contains a promoter sequence which facilitates the efficient transcription of the inserted genetic sequence of the host. The expression vector typically contains an origin of replication, a promoter, as well as specific nucleic acid sequences that allow phenotypic selection of the transformed cells.

Fc region: The constant region of an antibody excluding the first heavy chain constant domain. Fc region generally refers to the last two heavy chain constant domains of IgA, IgD, and IgG, and the last three heavy chain constant domains of IgE and IgM. An Fc region may also include part or all of the flexible hinge N-terminal to these domains. For IgA and IgM, an Fc region may or may not include the tailpiece, and may or may not be bound by the J chain. For IgG, the Fc region is typically understood to include immunoglobulin domains Cγ2 and Cγ3 and optionally the lower part of the hinge between Cγ1 and Cγ2. Although the boundaries of the Fc region may vary, the human IgG heavy chain Fc region is usually defined to include residues following C226 or P230 to the Fc carboxyl-terminus, wherein the numbering is according to Kabat. For IgA, the Fc region includes immunoglobulin domains Cα2 and Cα3 and optionally the lower part of the hinge between Cα1 and Cα2.

Fusion protein: A protein comprising at least a portion of two different (heterologous) proteins.

Heterologous: Originating from a different genetic source. A nucleic acid molecule that is heterologous to a cell originated from a genetic source other than the cell in which it is expressed. In one specific, non-limiting example, a heterologous nucleic acid molecule encoding a protein, such as an scFv, is expressed in a cell, such as a mammalian cell. Methods for introducing a heterologous nucleic acid molecule in a cell or organism are well known in the art, for example transformation with a nucleic acid, including electroporation, lipofection, particle gun acceleration, and homologous recombination.

Host cell: Cells in which a vector can be propagated and its DNA expressed. The cell may be prokaryotic or eukaryotic. The term also includes any progeny of the subject host cell. It is understood that all progeny may not be identical to the parental cell since there may be mutations that occur during replication. However, such progeny are included when the term “host cell” is used.

Immune complex: The binding of antibody (such as a single-domain monoclonal antibody disclosed herein) to a soluble antigen forms an immune complex. The formation of an immune complex can be detected through conventional methods, for instance immunohistochemistry, immunoprecipitation, flow cytometry, immunofluorescence microscopy, ELISA, immunoblotting (for example, Western blot), magnetic resonance imaging, CT scans, radiography, and affinity chromatography.

Inhibiting or treating a disease: Inhibiting the full development of a disease or condition, for example, in a subject who is at risk for a disease such as a SARS-COV-2 infection. “Treatment” refers to a therapeutic intervention that ameliorates a sign or symptom of a disease or pathological condition after it has begun to develop. The term “ameliorating,” with reference to a disease or pathological condition, refers to any observable beneficial effect of the treatment. Inhibiting a disease can include preventing or reducing the risk of the disease, such as preventing or reducing the risk of viral infection. The beneficial effect can be evidenced, for example, by a delayed onset of clinical symptoms of the disease in a susceptible subject, a reduction in severity of some or all clinical symptoms of the disease, a slower progression of the disease, a reduction in the viral load, an improvement in the overall health or well-being of the subject, or by other parameters that are specific to the particular disease. A “prophylactic” treatment is a treatment administered to a subject who does not exhibit signs of a disease or exhibits only early signs for the purpose of decreasing the risk of developing pathology.

The term “reduces” is a relative term, such that an agent reduces a disease or condition if the disease or condition is quantitatively diminished following administration of the agent, or if it is diminished following administration of the agent, as compared to a reference agent. Similarly, the term “prevents” does not necessarily mean that an agent completely eliminates the disease or condition, so long as at least one characteristic of the disease or condition is eliminated. Thus, a composition that reduces or prevents an infection, can, but does not necessarily completely, eliminate such an infection, so long as the infection is measurably diminished, for example, by at least about 50%, such as by at least about 70%, or about 80%, or even by about 90% of the infection in the absence of the agent, or in comparison to a reference agent.

Isolated: A biological component (such as a nucleic acid, peptide, protein or protein complex, for example an antibody) that has been substantially separated, produced apart from, or purified away from other biological components in the cell of the organism in which the component naturally occurs, that is, other chromosomal and extra-chromosomal DNA and RNA, and proteins. Thus, isolated nucleic acids, peptides and proteins include nucleic acids and proteins purified by standard purification methods. The term also embraces nucleic acids, peptides and proteins prepared by recombinant expression in a host cell, as well as, chemically synthesized nucleic acids. An isolated nucleic acid, peptide or protein, for example an antibody, can be at least 50%, at least 60%, at least 70%, at least 80%, at least 90%, at least 95%, at least 96%, at least 97%, at least 98%, or at least 99% pure.

Linker: A bi-functional molecule that can be used to link two molecules into one contiguous molecule, for example, to link a detectable marker to an antibody. Non-limiting examples of peptide linkers include glycine-serine linkers.

The terms “conjugating,” “joining,” “bonding,” or “linking” can refer to making two molecules into one contiguous molecule; for example, linking two polypeptides into one contiguous polypeptide, or covalently attaching an effector molecule or detectable marker radionuclide or other molecule to a polypeptide, such as an scFv. The linkage can be either by chemical or recombinant means. “Chemical means” refers to a reaction between the antibody moiety and the effector molecule such that there is a covalent bond formed between the two molecules to form one molecule.

Nucleic acid (molecule or sequence): A deoxyribonucleotide or ribonucleotide polymer or combination thereof including without limitation, cDNA, mRNA, genomic DNA, and synthetic (such as chemically synthesized) DNA or RNA. The nucleic acid can be double stranded (ds) or single stranded (ss). Where single stranded, the nucleic acid can be the sense strand or the antisense strand. Nucleic acids can include natural nucleotides (such as A, T/U, C, and G), and can include analogs of natural nucleotides, such as labeled nucleotides.

“cDNA” refers to a DNA that is complementary or identical to an mRNA, in either single stranded or double stranded form.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

Operably linked: A first nucleic acid sequence is operably linked with a second nucleic acid sequence when the first nucleic acid sequence is placed in a functional relationship with the second nucleic acid sequence. For instance, a promoter, such as the CMV promoter, is operably linked to a coding sequence if the promoter affects the transcription or expression of the coding sequence. Generally, operably linked DNA sequences are contiguous and, where necessary to join two protein-coding regions, in the same reading frame.

Pharmaceutically acceptable carriers: The pharmaceutically acceptable carriers of use are conventional. Remington: The Science and Practice of Pharmacy, 22^{nd} ed., London, UK: Pharmaceutical Press, 2013, describes compositions and formulations suitable for pharmaceutical delivery of the disclosed agents.

In general, the nature of the carrier will depend on the particular mode of administration being employed. For instance, parenteral formulations usually include injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, added preservatives (such as non-natural preservatives), and pH buffering agents and the like, for example sodium acetate or sorbitan monolaurate. In particular examples, the pharmaceutically acceptable carrier is sterile and suitable for parenteral administration to a subject for example, by injection. In some embodiments, the active agent and pharmaceutically acceptable carrier are provided in a unit dosage form such as a pill or in a selected quantity in a vial. Unit dosage forms can include one dosage or multiple dosages (for example, in a vial from which metered dosages of the agents can selectively be dispensed).

Polypeptide: A polymer in which the monomers are amino acid residues that are joined together through amide bonds. When the amino acids are alpha-amino acids, either the L-optical isomer or the D-optical isomer can be used, the L-isomers being preferred. The terms “polypeptide” or “protein” as used herein are intended to encompass any amino acid sequence and include modified sequences such as glycoproteins. A polypeptide includes both naturally occurring proteins, as well as those that are recombinantly or synthetically produced. A polypeptide has an amino terminal (N-terminal) end and a carboxy-terminal end. In some embodiments, the polypeptide is a disclosed antibody or a fragment thereof.

Purified: The term purified does not require absolute purity; rather, it is intended as a relative term. Thus, for example, a purified peptide preparation is one in which the peptide or protein (such as an antibody) is more enriched than the peptide or protein is in its natural environment, such as within a cell. In one embodiment, a preparation is purified such that the protein or peptide represents at least 50% of the total peptide or protein content of the preparation.

Recombinant: A recombinant nucleic acid is one that has a sequence that is not naturally occurring or has a sequence that is made by an artificial combination of two otherwise separated segments of sequence. This artificial combination can be accomplished by chemical synthesis or, more commonly, by the artificial manipulation of isolated segments of nucleic acids, for example, by genetic engineering techniques. A recombinant protein is one that has a sequence that is not naturally occurring or has a sequence that is made by an artificial combination of two otherwise separated segments of sequence. In several implementations, a recombinant protein is encoded by a heterologous (for example, recombinant) nucleic acid that has been introduced into a host cell, such as a bacterial or eukaryotic cell. The nucleic acid can be introduced, for example, on an expression vector having signals capable of expressing the protein encoded by the introduced nucleic acid or the nucleic acid can be integrated into the host cell chromosome.

SARS-COV-2: A coronavirus of the genus betacoronavirus that first emerged in humans in 2019. This virus is also known as Wuhan coronavirus, 2019-nCOV, or 2019 novel coronavirus. SARS-COV-2 is a positive-sense, single stranded RNA virus that has emerged as a fatal cause of severe acute respiratory infection. The viral genome is capped, polyadenylated, and covered with nucleocapsid proteins. The SARS-COV-2 virion includes a viral envelope with large spike glycoproteins. The SARS-COV-2 genome, like most coronaviruses, has a common genome organization with the replicase gene included in the 5′-two thirds of the genome, and structural genes included in the 3′-third of the genome. The SARS-COV-2 genome encodes the canonical set of structural protein genes in the order 5′-spike(S)-envelope (E)-membrane (M) and nucleocapsid (N)-3′.

The term “SARS-COV-2” includes variants thereof, such as, but not limited to, alpha (B.1.1.7 and Q lineages); beta (B.1.351 and descendent lineages); delta (B.1.617.2 and AY lineages); gamma (P.1 and descendent lineages); epsilon (B.1.427 and B.1.429); eta (B.1.525); iota (B.1.526); kappa (B.1.617.1); 1.617.3; mu (B.1.621, B.1.621.1), zeta (P.2), and omicron (B.1.1.529).

Symptoms of SARS-COV-2 infection include fever and respiratory illness, such as dry cough and shortness of breath. Cases of severe infection can progress to severe pneumonia, multi-organ failure, and death. The time from exposure to onset of symptoms is approximately 2 to 14 days.

Standard methods for detecting viral infection may be used to detect SARS-COV-2 infection, including but not limited to, assessment of patient symptoms and background and genetic tests such as reverse transcription-polymerase chain reaction (rRT-PCR). The test can be done on patient samples such as respiratory or blood samples.

SARS Spike(S) protein: A class I fusion glycoprotein initially synthesized as a precursor protein of approximately 1256 amino acids in size for SARS-COV, and 1273 for SARS-COV-2. Individual precursor S polypeptides form a homotrimer and undergo glycosylation within the Golgi apparatus as well as processing to remove the signal peptide, and cleavage by a cellular protease between approximately position 679/680 for SARS-COV, and 685/686 for SARS-COV-2, to generate separate S1 and S2 polypeptide chains, which remain associated as S1/S2 protomers within the homotrimer and is therefore a trimer of heterodimers. The S1 subunit is distal to the virus membrane and contains the N-terminal domain (NTD) and the receptor-binding domain (RBD) that is believed to mediate virus attachment to its host receptor. The S2 subunit is believed to contain the fusion protein machinery, such as the fusion peptide, two heptad-repeat sequences (HR1 and HR2) and a central helix typical of fusion glycoproteins, a transmembrane domain, and the cytosolic tail domain.

Scaffold: The four framework regions of a monoclonal antibody, such as a synthetic single-domain monoclonal antibody disclosed herein. In some implementations, the scaffold of the single-domain monoclonal antibody comprises the amino acid sequences of FR1, FR2, FR3 and FR4, set forth as SEQ ID NOs: 1, 2, 3 and 4, respectively. The scaffold can also include variants of FR1, FR2, FR3 and FR4, such as variants having 1-5 conservative amino acid substitutions, such as 1, 2, 3, 4 or 5 conservative amino acid substitutions with respect to SEQ ID NOs: 1, 2, 3 and/or 4.

Sequence identity: The identity between two or more nucleic acid sequences, or two or more amino acid sequences, is expressed in terms of the percentage identity between the sequences. Sequence identity can be measured in terms of percentage identity; the higher the percentage, the more identical the sequences. Homologs and variants of a VL or a VH of an antibody that specifically binds a target antigen are typically characterized by possession of at least about 75% sequence identity, for example at least about 80%, 85%, 90%, 91%, 92%, 93%, 94%, 95%, 96%, 97%, 98% or 99% sequence identity counted over the full-length alignment with the amino acid sequence of interest.

Any suitable method may be used to align sequences for comparison. Non-limiting examples of programs and alignment algorithms are described in: Smith and Waterman, Adv. Appl. Math. 2 (4): 482-489, 1981; Needleman and Wunsch, J. Mol. Biol. 48 (3): 443-453, 1970; Pearson and Lipman, Proc. Natl. Acad. Sci. U.S.A. 85 (8): 2444-2448, 1988; Higgins and Sharp, Gene, 73 (1): 237-244, 1988; Higgins and Sharp, Bioinformatics, 5 (2): 151-3, 1989; Corpet, Nucleic Acids Res. 16 (22): 10881-10890, 1988; Huang et al. Bioinformatics, 8 (2): 155-165, 1992; and Pearson, Methods Mol. Biol. 24:307-331, 1994., Altschul et al., J. Mol. Biol. 215 (3): 403-410, 1990, presents a detailed consideration of sequence alignment methods and homology calculations. The NCBI Basic Local Alignment Search Tool (BLAST) (Altschul et al., J. Mol. Biol. 215 (3): 403-410, 1990) is available from several sources, including the National Center for Biological Information and on the Internet, for use in connection with the sequence analysis programs blastp, blastn, blastx, tblastn, and tblastx. Blastn is used to compare nucleic acid sequences, while blastp is used to compare amino acid sequences. Additional information can be found at the NCBI web site.

Generally, once two sequences are aligned, the number of matches is determined by counting the number of positions where an identical nucleotide or amino acid residue is present in both sequences. The percent sequence identity between the two sequences is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100.

Specifically bind: When referring to an antibody, refers to a binding reaction which determines the presence of a target protein in the presence of a heterogeneous population of proteins and other biologics. Thus, under designated conditions, an antibody binds preferentially to a particular target protein, peptide or polysaccharide (such as an antigen present on the surface of a pathogen, for example a coronavirus spike protein) and does not bind in a significant amount to other proteins present in the sample or subject. With regard to a spike protein, the epitope may be present on SARS-COV-2 spike protein, such that the antibody binds to the spike protein on both types of virus, but does not bind to other proteins. Specific binding can be determined by standard methods. See Harlow & Lane, Antibodies, A Laboratory Manual, 2^{nd} ed., Cold Spring Harbor Publications, New York (2013), for a description of immunoassay formats and conditions that can be used to determine specific immunoreactivity.

With reference to an antibody-antigen complex, specific binding of the antigen and antibody has a K_{D} of less than about 10⁻⁷ Molar, such as less than about 10⁻⁸ Molar, 10⁻⁹, or even less than about 10⁻¹⁰ Molar. K_{D} refers to the dissociation constant for a given interaction, such as a polypeptide ligand interaction or an antibody antigen interaction. For example, for the bimolecular interaction of an antibody or antigen binding fragment and an antigen it is the concentration of the individual components of the bimolecular interaction divided by the concentration of the complex.

An antibody that specifically binds to an epitope on a coronavirus spike protein is an antibody that binds substantially to the coronavirus spike protein, such as the NTD or RBD of a spike protein from SARS-COV-2, including viruses, substrate to which the spike protein is attached, or the protein in a biological specimen. It is, of course, recognized that a certain degree of non-specific interaction may occur between an antibody and a non-target. Typically, specific binding results in a much stronger association between the antibody and a spike protein than between the antibody and other different coronavirus proteins (such as MERS), or from non-coronavirus proteins. Specific binding typically results in greater than 2-fold, such as greater than 5-fold, greater than 10-fold, or greater than 100-fold increase in amount of bound antibody (per unit time) to a protein including the epitope or cell or tissue expressing the target epitope as compared to a protein or cell or tissue lacking this epitope. Specific binding to a protein under such conditions requires an antibody that is selected for its specificity for a particular protein. A variety of immunoassay formats are appropriate for selecting antibodies or other ligands specifically immunoreactive with a particular protein. For example, solid-phase ELISA immunoassays are routinely used to select monoclonal antibodies specifically immunoreactive with a protein.

Subject: Living multi-cellular vertebrate organisms, a category that includes human and non-human mammals, such as non-human primates, pigs, camels, bats, sheep, cows, dogs, cats, rodents, and the like. In an example, a subject is a human. In a particular example, the subject is a human. In an additional example, a subject is selected that is in need of inhibiting a SARS-COV-2 infection. For example, the subject is either uninfected and at risk of the SARS-COV-2 infection or is infected and in need of treatment.

Synthetic: Produced by artificial means in a laboratory, for example a synthetic nucleic acid or protein (for example, an antibody) can be chemically synthesized in a laboratory.

Synthetic single-domain monoclonal antibody library: In the context of the present disclosure, the term “synthetic single-domain monoclonal antibody library” encompasses nucleic acid libraries comprising synthetic single-domain antibody coding sequences with a high degree of diversity among CDR sequences, and optionally a cloning vector or expression vector. A synthetic single-domain monoclonal antibody library may further include any transformed host cells or organisms containing the nucleic acid libraries, such as bacterial, yeast or filamentous fungi, or mammalian cells transformed with the nucleic acid libraries, or bacteriophages or viruses containing the nucleic acid libraries. This term may further include the corresponding mixture of diverse antibodies encoded by the nucleic acid library.

Transformed: A transformed cell is a cell into which a nucleic acid molecule has been introduced by molecular biology techniques. As used herein, the term transformed and the like (e.g., transformation, transfection, transduction, etc.) encompasses all techniques by which a nucleic acid molecule might be introduced into such a cell, including transduction with viral vectors, transformation with plasmid vectors, and introduction of DNA by electroporation, lipofection, and particle gun acceleration.

Vector: An entity containing a nucleic acid molecule (such as a DNA or RNA molecule) bearing a promoter(s) that is operationally linked to the coding sequence of a protein of interest and can express the coding sequence. Non-limiting examples include a naked or packaged (lipid and/or protein) DNA, a naked or packaged RNA, a subcomponent of a virus or bacterium or other microorganism that may be replication-incompetent, or a virus or bacterium or other microorganism that may be replication-competent. A vector is sometimes referred to as a construct. Recombinant DNA vectors are vectors having recombinant DNA. A vector can include nucleic acid sequences that permit it to replicate in a host cell, such as an origin of replication. A vector can also include one or more selectable marker genes and other genetic elements. Viral vectors are recombinant nucleic acid vectors having at least some nucleic acid sequences derived from one or more viruses. In some implementations, a viral vector comprises a nucleic acid molecule encoding a disclosed antibody or antigen binding fragment that specifically binds to a coronavirus spike protein and neutralizes the coronavirus. In some implementations, the viral vector can be an adeno-associated virus (AAV) vector.

Under conditions sufficient for: A phrase that is used to describe any environment that permits a desired activity.

### III. Introduction

Single-domain monoclonal antibodies, also known as “nanobodies,” are a unique class of heavy chain-only antibodies present in Camelidae (V_{H}H) and some shark species. V_{H}H and shark variable new antigen receptor (V_{NAR}) antibodies are one order of magnitude smaller than their full-length IgG counterparts. Advantages of using these types of single-domain monoclonal antibodies as anti-viral biologics include convenient bulk production at multi-kilogram scale in prokaryotic systems, long shelf-life, and greater permeability in tissues (Dumoulin et al., Protein Sci 11, 500-515, 2002; Hussack et al., PLOS One 6, e28218, 2011). Single-domain monoclonal antibodies can be administered orally (e.g., V565 for GIT activity) or through inhalation (e.g., ALX-0171), both of which routes are beneficial for the treatment of COVID-19 (Arezumand et al., Front Immunol 8, 1746, 2017; Nambulli et al., Science Advances 7, eabh0319, 2021).

Disclosed herein is a rapid and efficient method for identification of neutralizing single-domain monoclonal antibodies directed against a target of interest, such as the SARS-COV-2 spike protein, through phage display. Synthetic libraries were constructed by combining a humanized single-domain monoclonal antibody framework with randomized complementarity determining regions (CDRs) to diversify antigen recognition. Among ten enriched RBD binders, RBD-1-2G was the most potent with an IC₅₀ of 490 nM against SARS-COV-2 pseudotyped particles. Constructing bi- or tri-valent formats of the RBD-1-2G antibody resulted in improved affinity and neutralization potency against pseudotyped particles and authentic SARS-COV-2 virus. Furthermore, cryo-electron microscopy (cryo-EM) structures for four single-domain monoclonal antibodies in complex with a stabilized SARS-COV-2 S-protein ectodomain (Wrapp et al., Science 367, 1260-1263, 2020) revealed two distinct binding modes. The epitope for ‘Group 1’ single-domain monoclonal antibodies overlaps the receptor binding motif (RBM) at the distal end of the RBD. ‘Group 2’ binders target epitopes on the flat area proximal to the N-terminal domain of the adjacent monomer. This binding site was found to not overlap with the RBM and failed to inhibit ACE binding. Furthermore, cryo-EM showed that three copies of RBD-1-2G were capable of binding to the same spike trimer. The RBD-1-2G antibody was capable of neutralizing pseudotyped particles containing the N501Y mutation. Molecular dynamics simulation provides a basis for single-domain monoclonal antibody exclusively focused on the spike-ACE2 interface indicating that CDR3 is the most important region involved in the neutralization of the WT SARS-CoV-2 and the B.1.1.7 alpha variant (N501Y).

The studies disclosed herein demonstrated that panning synthetic humanized single-domain monoclonal antibody libraries is an efficient, fast-track method to identify potent neutralizing antibodies that may have strong therapeutic and prophylactic potential. This approach could greatly assist in the intervention of prevailing and emerging infectious diseases, such as COVID-19.

### IV. Synthetic Single-Domain Monoclonal Antibody Libraries

The present disclosure provides a method of making a synthetic single-domain monoclonal antibody library. In some implementations, the method includes introducing a diversity of nucleic acid molecules encoding complementarity determining region 1 (CDR1), CDR2 and CDR3 sequences between respective framework (FR) coding regions of a synthetic single-domain monoclonal antibody to generate nucleic acid molecules encoding a diversity of synthetic single-domain monoclonal antibodies with the same synthetic single-domain monoclonal antibody scaffold amino acid sequence. In some implementations, the synthetic single-domain monoclonal antibody scaffold includes a FR1 sequence comprising SEQ ID NO: 1, a FR2 sequence comprising SEQ ID NO: 2, a FR3 sequence comprising SEQ ID NO: 3 and a FR4 sequence comprising SEQ ID NO: 4.

In some implementations, CDR1 is 5 to 8 residues (5, 6, 7 or 8 residues) in length and the amino acid residues of the CDR1 sequence are determined according to the following rules: CDR1 position 1 is glycine (G); CDR1 position 2 is asparagine (N), serine(S), threonine (T) or tyrosine (Y); CDR1 position 3 is isoleucine (I); CDR1 position 4 is phenylalanine (F) or S; CDR1 position 5 is Y, G, aspartate (D), alanine (A), arginine (R), S, valine (V), F, leucine (L), T, glutamate (E), proline (P), tryptophan (W), histidine (H), lysine (K), I, methionine (M), N or glutamine (Q); and CDR1 positions 6, 7 and/or 8, if present, are individually selected from Y, G, D, A, R, S, V, F, L, T, E, P, W, H, K, I, M, N and Q. In some examples, the amino acid composition of each of position 5, position 6 if present, position 7 if present, and/or position 8 if present, includes 13% Y, 12% G, 10% D, 10% A, 8% R, 8% S, 5% V, 5% F, 4% L, 4% T, 3% E, 3% P, 3% W, 2% H, 2% K, 2% 1, 2% M, 2% N and 2% Q.

In some implementations, CDR2 is 9 residues in length and the amino acid residues of the CDR2 sequence are determined according to the following rules: CDR2 position 1 is I; CDR2 position 2 is A, D, G, N, S or T; CDR2 position 3 is Y, G, D, A, R, S, V, F, L, T, E, P, W, H, K, I, M, N or Q; CDR2 position 4 is Y, G, D, A, R, S, V, F, L, T, E, P, W, H, K, I, M, N or Q; CDR2 position 5 is G; CDR2 position 6 is A, G, S, or T; CDR2 position 7 is I, N, S or T; CDR2 position 8 is T; and CDR2 position 9 is N or Y. In some examples, the amino acid composition of each of position 3 and position 4 includes 13% Y, 12% G, 10% D, 10% A, 8% R, 8% S, 5% V, 5% F, 4% L, 4% T, 3% E, 3% P, 3% W, 2% H, 2% K, 2% 1, 2% M, 2% N and 2% Q.

In some implementations, CDR3 is 14 to 20 amino acids in length (14, 15, 16, 17, 18, 19, 20, 21, 22, 23 or 24 amino acids in length) and each position is individually selected from Y, G, D, A, R, S, V, F, L, T, E, P, W, H, K, I, M, N and Q. In some examples, the amino acid composition of each position of CDR3 includes 13% Y, 12% G, 10% D, 10% A, 8% R, 8% S, 5% V, 5% F, 4% L, 4% T, 3% E, 3% P, 3% W, 2% H, 2% K, 2% 1, 2% M, 2% N and 2% Q.

Also provided herein is a synthetic single-domain monoclonal antibody library obtainable by the method disclosed herein. In some implementations, the synthetic single-domain monoclonal antibody library includes at least 10⁸, 10⁹, 10¹⁰ or 10¹¹ unique antibody sequences. In some examples, the synthetic single-domain monoclonal antibody library includes at least 10¹⁰ unique antibody sequences.

Further provided herein is a screening method for identifying a synthetic single-domain monoclonal antibody that binds to a target of interest. In some implementations, the screening method includes the use of a synthetic single-domain antibody library disclosed herein. In some examples, the target of interest is a microbial antigen, such as a viral antigen, bacterial antigen, fungal antigen or a parasite antigen. In specific examples, the viral antigen is a SARS-COV-2 antigen, such as a spike protein. In other examples, the target of interest is a tumor antigen. In some implementations, the screening method is a phage display method.

Also provided herein are single-domain monoclonal antibodies having the following formula: FR1-CDR1-FR2-CDR2-FR3-CDR3-FR4, wherein the amino acid sequence of FR1 comprises SEQ ID NO: 1; the amino acid sequence of FR2 comprises SEQ ID NO: 2; the amino acid sequence of FR3 comprises SEQ ID NO: 3; and the amino acid sequence of FR4 comprises SEQ ID NO: 4.

In some implementations of the single-domain monoclonal antibodies, CDR1 is 5 to 8 (5, 6, 7 or 8) residues in length and the amino acid residues of the CDR1 sequence are determined according to the following rules: CDR1 position 1 is G; CDR1 position 2 is N, S, T or Y; CDR1 position 3 is I; CDR1 position 4 is F or S; CDR1 position 5 is Y, G, D, A, R, S, V, F, L, T, E, P, W, H, K, I, M, N or Q; and CDR1 positions 6, 7 and/or 8, if present, are randomly selected from Y, G, D, A, R, S, V, F, L, T, E, P, W, H, K, I, M, N or Q. In some examples, the amino acid composition of each of position 5, position 6 if present, position 7 if present, and/or position 8 if present, includes 13% Y, 12% G, 10% D, 10% A, 8% R, 8% S, 5% V, 5% F, 4% L, 4% T, 3% E, 3% P, 3% W, 2% H, 2% K, 2% I, 2% M, 2% N and 2% Q.

In some implementations, CDR2 is 9 residues in length and the amino acid residues of the CDR2 sequence are determined according to the following rules: CDR2 position 1 is I; CDR2 position 2 is A, D, G, N, S or T; CDR2 position 3 is Y, G, D, A, R, S, V, F, L, T, E, P, W, H, K, I, M, N or Q; CDR2 position 4 is Y, G, D, A, R, S, V, F, L, T, E, P, W, H, K, I, M, N or Q; CDR2 position 5 is G; CDR2 position 6 is A, G, S, or T; CDR2 position 7 is I, N, S or T; CDR2 position 8 is T; and CDR2 position 9 is N or Y. In some examples, the amino acid composition of each of position 3 and position 4 includes 13% Y, 12% G, 10% D, 10% A, 8% R, 8% S, 5% V, 5% F, 4% L, 4% T, 3% E, 3% P, 3% W, 2% H, 2% K, 2% 1, 2% M, 2% N and 2% Q.

In some implementations, CDR3 is 14 to 20 amino acids in length (14, 15, 16, 17, 18, 19, 20, 21, 22, 23 or 24 amino acids in length) and each position is individually selected from Y, G, D, A, R, S, V, F, L, T, E, P, W, H, K, I, M, N and Q. In some examples, the amino acid composition of each position of CDR3 includes 13% Y, 12% G, 10% D, 10% A, 8% R, 8% S, 5% V, 5% F, 4% L, 4% T, 3% E, 3% P, 3% W, 2% H, 2% K, 2% 1, 2% M, 2% N and 2% Q.

Further provided are nucleic acid molecules encoding the single-domain monoclonal antibodies disclosed herein. In some implementations, the nucleic acid molecule is operably linked to a promoter, such as a heterologous promoter. Also provided are vectors that include a disclosed nucleic acid molecule and isolated host cells that include a disclosed nucleic acid molecule or vector.

### A. Introducing CDR Diversity in the Single-Domain Antibody Scaffold

Methods for generating CDR diversity for antibody libraries, such as by random or directed synthesis of CDR coding sequences and cloning into corresponding framework sequences, have been described (e.g., US 2016/0237142; and Lindner et al., Molecules 16:1625-1641, 2011). Similarly, the synthetic single-domain monoclonal antibody libraries disclosed herein were generated by introducing high CDR diversity into a selected scaffold sequence (U.S. Pat. No. 10,919,980).

In some implementations, the position of each amino acid sequence of synthetic CDR1 and synthetic CDR2 is rationally designed to mimic natural diversity of CDRs found in the human repertoire. In some examples, cysteines are avoided because of their thiol groups which could interfere with intracellular expression and functionality.

The length of the CDR3 sequence may influence the binding potential of the antibody to different epitope shapes, particularly binding to an epitope in a protein cavity. Therefore, the disclosed single-domain monoclonal antibody libraries vary in the length of CDR3 sequences. In some examples, the CDR3 is 14 to 20 amino acids in length, such as 14, 15, 16, 17, 18, 19 or 20 amino acids in length.

In some implementations, CDR1 is 5 to 8 amino acids in length, such as 5, 6, 7 or 8 amino acids in length. In some examples, the amino acid residues of CDR1 are selected according to the following rules: CDR1 position 1 is G; CDR1 position 2 is N, S, T or Y; CDR1 position 3 is I; CDR1 position 4 is F or S; CDR1 position 5 is Y, G, D, A, R, S, V, F, L, T, E, P, W, H, K, I, M, N or Q; and CDR1 positions 6, 7 and/or 8, if present, are randomly selected from Y, G, D, A, R, S, V, F, L, T, E, P, W, H, K, I, M, N or Q. In some examples, the amino acid composition of each of position 5, position 6 if present, position 7 if present, and/or position 8 if present, includes 13% Y, 12% G, 10% D, 10% A, 8% R, 8% S, 5% V, 5% F, 4% L, 4% T, 3% E, 3% P, 3% W, 2% H, 2% K, 2% 1, 2% M, 2% N and 2% Q (see FIG. 1A).

In some implementations, CDR2 is 9 residues in length and the amino acid residues of the CDR2 sequence are determined according to the following rules: CDR2 position 1 is I; CDR2 position 2 is A, D, G, N, S or T; CDR2 position 3 is Y, G, D, A, R, S, V, F, L, T, E, P, W, H, K, I, M, N or Q; CDR2 position 4 is Y, G, D, A, R, S, V, F, L, T, E, P, W, H, K, I, M, N or Q; CDR2 position 5 is G; CDR2 position 6 is A, G, S, or T; CDR2 position 7 is I, N, S or T; CDR2 position 8 is T; and CDR2 position 9 is N or Y. In some examples, the amino acid composition of each of position 3 and position 4 includes 13% Y, 12% G, 10% D, 10% A, 8% R, 8% S, 5% V, 5% F, 4% L, 4% T, 3% E, 3% P, 3% W, 2% H, 2% K, 2% 1, 2% M, 2% N and 2% Q.

In some implementations, CDR3 is 14 to 20 amino acids in length (14, 15, 16, 17, 18, 19, 20, 21, 22, 23 or 24 amino acids in length) and each position is individually selected from Y, G, D, A, R, S, V, F, L, T, E, P, W, H, K, I, M, N and Q. In some examples, the amino acid composition of each position of CDR3 includes 13% Y, 12% G, 10% D, 10% A, 8% R, 8% S, 5% V, 5% F, 4% L, 4% T, 3% E, 3% P, 3% W, 2% H, 2% K, 2% 1, 2% M, 2% N and 2% Q.

The above rules are used as a guide for generating the single-domain monoclonal antibody libraries disclosed herein. However, other libraries with different amino acid diversity rules are also contemplated, as long as they contain the synthetic single-domain monoclonal antibody scaffold disclosed herein (SEQ ID NOs: 1-4 or variants thereof).

In particular implementations, only a significant proportion of the clones of the library strictly follow the above rules for CDR composition. For example, statistically, at least 50%, at least 60%, at least 70%, at least 80% or at least 90% of the clones of the library follow the above rules of composition of amino acid residues in CDR1, CDR2 and CDR3 positions.

In some implementations, to follow the above-listed rules for amino acid positions, and to avoid the occurrence of an in-frame stop codon or a cysteine residue, or to reduce occurrence of a frameshift, advanced gene synthesis approaches are used. These methods include, but are not limited to, double strand DNA triple blocks (see, e.g., Van den Brulle et al., Biotechniques 45 (3): 340-343, 2008), tri-nucleotide synthesis, or other codon-controlled and more generally position-controlled degenerate synthesis approaches.

In some implementations, codon bias is optimized, for example, for host cell species, for example, mammalian host cell expression, using well known methods. In some examples, the coding sequence is designed so that it does not contain undesired restriction sites, for example, restriction sites that are used for cloning the coding sequence into the appropriate cloning or expression vector.

The resulting diverse coding sequences are introduced into a suitable expression or cloning vectors for antibody libraries. In particular implementations, the expression vector is a plasmid. In other particular implementations, the expression vector is suitable for generating phage display libraries. Two different types of vectors may be used for generating phage display libraries: phagemid vectors and phage vectors.

Phagemids are derived from filamentous phage (Ff-phage-derived) vectors, containing the replication origin of a plasmid. The basic components of a phagemid primarily include a replication origin of a plasmid, a selective marker, an intergenic region (IG region, usually contains the packing sequence and replication origin of minus and plus strands), a gene of a phage coat protein, restriction enzyme recognition sites, a promoter and a DNA segment encoding a signal peptide. Additionally, a molecular tag can be included to facilitate screening of a phagemid-based library. Phagemids can be converted to filamentous phage particles with the same morphology as Ff phage by co-infection with the helper phages, such as R408, M13KO7 and VCSM13 (Stratagene). One example of a phage vector is fd-tet (Zacher et al., Gene 9:127-140, 1980), which consists of a fd-phage genome and a segment of Tn10 inserted near the phage genome origin of replication. Examples of promoters for use in phagemid vectors include, but are not limited to, PlacZ and PT7, examples of signal peptides include, but are not limited to, pelB leader, gIII, CAT leader, SRP and OmpA signal peptide.

Other phage-display methods include those using lytic phages, such as T4 or T7. Vectors other than phages may also be used to generate display libraries, including vectors for bacterial cell display (Daugherty et al., Protein Eng 12 (7): 613-621, 1999; Georgiou et al., Nat Biotechnol 15 (1): 29-34, 1997), yeast cell display (Boder and Wittrup, Nat Biotechnol 15 (6): 553-557, 1997), ribosome display (Zahnd et al., Nat Methods 4 (3): 269-279, 2007), DNA display (Eldridge et al., Protein Eng Des Sel 22 (11): 691-698, 2009), and surface display on mammalian cells (Rode et al., Biotechniques 21 (4): 650, 652-3, 655-6, 658, 1996). Non-display methods, such as yeast two-hybrid, may also be used to select relevant binders from the library (Visintin et al., Proc Natl Acad Sci USA 96:11723-11728, 1999).

In some implementations, to avoid generating empty vectors, positive selection of recombinant coding sequence in the cloning vectors bearing a suicide gene is used (see, e.g., Bernard, BioTechniques 21 (2) 320-323, 1996).

In some examples, the diversity as calculated by all possible combinations of CDR amino acid residues as designed for generating the antibody library is at least 10⁹, at least 10¹⁰, at least 10¹¹ or at least 10¹² unique sequences.

### B. Synthetic Single-Domain Monoclonal Antibody Libraries and Use Thereof

Also provided herein are synthetic single-domain monoclonal antibody libraries produced by the methods disclosed herein. In some implementations, the synthetic single-domain monoclonal antibody library includes at least 10⁸, at least 10⁹, at least 10¹⁰ or at least 10¹¹ unique antibody sequences. In some examples, the synthetic single-domain monoclonal antibody library includes at least 10¹⁰ unique antibody sequences.

In some implementations, the synthetic single-domain monoclonal antibody is used in a screening method, such as to identify a single-domain monoclonal antibody that specifically binds a target of interest. Any known screening methods for identifying binders with specific affinity to a target of interest may be used with the synthetic single-domain monoclonal antibody libraries disclosed herein. Such methods include, but are not limited to, phage display technologies, bacterial cell display, yeast cell display, mammalian cell display or ribosome display. In particular examples, the screening method is the phage display.

In some implementations, the target of interest is a therapeutic target, and the synthetic single-domain monoclonal antibody library is used to identify synthetic single-domain antibodies with specific binding to the therapeutic target. In specific implementations, the target of interest is an antigen, or includes at least an antigenic determinant. For example, the target can be a saccharide or polysaccharide, a protein or glycoprotein, or a lipid. In one specific implementation, the target of interest is of plant, yeast, fungus, insect, mammalian or other eukaryote cell origin. In another specific implementation, the target of interest is of bacterial, protozoan or viral origin. In particular examples, the target is an antigen, such as a viral, bacterial, fungal or protozoan antigen. In specific non-limiting examples, the target is a SARS-COV-2 antigen such as a spike protein.

C. Single-Domain Monoclonal Antibodies Obtained from a Library

Further provided are single-domain monoclonal antibodies selected from a single-domain monoclonal antibody library disclosed herein. In view of the high diversity of synthetic single-domain monoclonal antibodies of the disclosed libraries, the skilled person can obtain synthetic single domain antibodies with high affinity and high specificity to a target of interest (such as an antigen, for example SARS-COV-2 spike protein), by conventional screening methods, such a phage display/panning.

The selected single-domain monoclonal antibody can optionally be further modified for generating appropriate antigen-binding properties. In particular, the CDR residues may be modified for example to increase the antibody affinity to the target of interest, improve its folding or its production, or lower its viscosity, using technologies known in the art (e.g., mutagenesis, affinity maturation).

In some implementations, provided herein are single-domain monoclonal antibodies having the following formula: FR1-CDR1-FR2-CDR2-FR3-CDR3-FR4, wherein the framework regions FR1, FR2, FR3, and FR4 are humanized llama framework sequences. In particular examples, the amino acid sequences of the framework regions FR1, FR2, FR3 and FR4 respectively comprise SEQ ID NOs: 1, 2, 3 and 4. In other examples, the framework sequences are functional variants of SEQ ID NOs: 1, 2, 3 and 4 having no more than 1, 2, 3, 4, or 5 conservative amino acid substitutions with respect to SEQ ID NOs: 1, 2, 3 and 4, while retaining specific binding to the target of interest. In yet other examples, the framework sequences are functional variants of SEQ ID NOs: 1, 2, 3 and 4 having at least 80%, at least 90%, at least 95%, at least 96%, at least 97%, at least 98% or at least 99% sequence identity to SEQ ID NOs: 1, 2, 3 and 4, respectively, while retaining specific binding to the target of interest. Typically, one or more amino acid residues within the framework regions can be replaced with other amino acid residues from the same side chain family, and the new variant be tested in binding and/or functional assays.

In some implementations, CDR1 is 5 to 8 amino acids in length, such as 5, 6, 7 or 8 amino acids in length. In some examples, the amino acid residues of CDR1 are selected according to the following rules: CDR1 position 1 is G; CDR1 position 2 is N, S, T or Y; CDR1 position 3 is I; CDR1 position 4 is F or S; CDR1 position 5 is Y, G, D, A, R, S, V, F, L, T, E, P, W, H, K, I, M, N or Q; and CDR1 positions 6, 7 and/or 8, if present, are randomly selected from Y, G, D, A, R, S, V, F, L, T, E, P, W, H, K, I, M, N or Q. In some examples, the amino acid composition of each of position 5, position 6 if present, position 7 if present, and/or position 8 if present, includes 13% Y, 12% G, 10% D, 10% A, 8% R, 8% S, 5% V, 5% F, 4% L, 4% T, 3% E, 3% P, 3% W, 2% H, 2% K, 2% 1, 2% M, 2% N and 2% Q (see FIG. 1A).

In some implementations, CDR2 is 9 residues in length and the amino acid residues of the CDR2 sequence are determined according to the following rules: CDR2 position 1 is I; CDR2 position 2 is A, D, G, N, S or T; CDR2 position 3 is Y, G, D, A, R, S, V, F, L, T, E, P, W, H, K, I, M, N or Q; CDR2 position 4 is Y, G, D, A, R, S, V, F, L, T, E, P, W, H, K, I, M, N or Q; CDR2 position 5 is G; CDR2 position 6 is A, G, S, or T; CDR2 position 7 is I, N, S or T; CDR2 position 8 is T; and CDR2 position 9 is N or Y. In some examples, the amino acid composition of each of position 3 and position 4 includes 13% Y, 12% G, 10% D, 10% A, 8% R, 8% S, 5% V, 5% F, 4% L, 4% T, 3% E, 3% P, 3% W, 2% H, 2% K, 2% 1, 2% M, 2% N and 2% Q (see FIG. 1A).

In some implementations, CDR3 is 14 to 20 amino acids in length (14, 15, 16, 17, 18, 19, 20, 21, 22, 23 or 24 amino acids in length) and each position is individually selected from Y, G, D, A, R, S, V, F, L, T, E, P, W, H, K, I, M, N and Q. In some examples, the amino acid composition of each position of CDR3 includes 13% Y, 12% G, 10% D, 10% A, 8% R, 8% S, 5% V, 5% F, 4% L, 4% T, 3% E, 3% P, 3% W, 2% H, 2% K, 2% 1, 2% M, 2% N and 2% Q (see FIG. 1A).

### D. Nucleic Acid Molecules

Also provided herein are nucleic acid molecules that encode a single-domain monoclonal antibody selected from a single-domain monoclonal antibody library disclosed herein. In some implementations, the nucleic acid molecule is operably linked to a promoter, such as a heterologous promoter. The nucleic acid can be present in whole cells, in a cell lysate, or can be nucleic acid in a partially purified or substantially pure form. In some examples, the nucleic acid molecule is DNA or RNA and may or may not contain intronic sequences.

In some implementations, the nucleic acid is a DNA molecule. The nucleic acid can be present in a vector, such as a phage display vector, or in a recombinant plasmid vector. In some examples, provided is an isolated nucleic acid molecule or a vector comprising at least one or more nucleic acid sequences encoding framework regions FR1, FR2, FR3 and FR4 of SEQ ID NOs: 1-4, or encoding corresponding variant sequences having at least 80%, at least 90%, at least 95%, at least 96%, at least 97%, at least 98% or at least 99% sequence identity to said SEQ ID NOs: 1-4.

Nucleic acid molecules encoding the single-domain monoclonal antibodies can be further manipulated by standard recombinant DNA techniques, for example to include a signal sequence for appropriate secretion in an expression system, a purification tag and/or cleavable tag for further purification steps. In these manipulations, a nucleic acid molecules (such as a DNA molecule) is operatively linked to another DNA molecule, or to a fragment encoding another protein, such as a purification/secretion tag or a flexible linker. The term “operatively linked” as used in this context, is intended to mean that the two DNA molecules are joined in a functional manner, for example, such that the amino acid sequences encoded by the two DNA molecules remain in-frame, or such that the protein is expressed under control of a desired promoter.

Further provided are isolated host cells suitable for the production of the single-domain monoclonal antibodies. In some implementations, the host cell of is a mammalian cell line. Also provided is a process for the production of a single-domain monoclonal antibody, comprising culturing the host cell under appropriate conditions for the production of single-domain monoclonal antibody, and isolating the single-domain monoclonal antibody.

Mammalian host cells for secreting the single-domain monoclonal antibodies of the disclosure include CHO, such as DHFR-CHO cells (described in Urlaub and Chasin, 1980, Proc. Natl. Acad. Sci. USA 77:4216-4220) used with a DHFR selectable marker (e.g., as described in Kaufman and Sharp, 1982 Mol. Biol. 159:601-621), NSO myeloma cells, the pFuse expression system from Invivogen (as described in Moutel et al., BMC Biotechnol 9:14, 2009), COS cells, SP2 cells or human cell lines, including PER-C6 cell lines, Crucell or HEK293 cells (Durocher et al., 2002, Nucleic Acids Res 30 (2): 9). When the nucleic acid molecules encoding the single-domain monoclonal antibodies are introduced into mammalian host cells, the antibodies are produced by culturing the host cells for a period of time sufficient to allow for expression of the recombinant polypeptides in the host cells or secretion of the recombinant polypeptides into the culture medium in which the host cells are grown and proper refolding to produce the single-domain monoclonal antibodies. The single-domain monoclonal antibodies can then be recovered from the culture medium using standard protein purification methods.

### V. Monoclonal Antibodies Specific for Coronavirus Spike Protein

Described herein are synthetic single-domain monoclonal antibodies that specifically bind SARS-COV-2 spike protein with high affinity. Single-domain monoclonal antibody RBD-1-2G exhibits potent neutralizing activity against SARS-COV-2 infection. This antibody was selected from a synthetic single-domain monoclonal antibody phage display library.

The amino acid sequence of RBD-1-2G is provided below. CDR sequences determined using the method of IMGT are indicated by bold underline. One of skill in the art could readily determine the CDR boundaries using an alternative numbering scheme, such as the Kabat or Chothia numbering scheme.

RBD-1-2G
(SEQ ID NO: 5)
EVQLVESGGGLVQPGGSLRLSCAASGFSSIVYMGW
FRQAPGKGRELVAAIDASGSTTNYPDSVEGRFTIS
RDNAKRMVYLQMNSLRAEDTAVYYCAIAYFTSPEY
VVSQGWGQGTQVTVSS

| Feature: | FR1 | CDR1 | FR2 | CDR2 | FR3 | CDR3 | FR4 |
|---|---|---|---|---|---|---|---|
| Residues: | 1-25 | 26-32 | 33-49 | 50-58 | 59-96 | 97-110 | 111-121 |

Provided herein are singe-domain monoclonal antibodies that bind (for example, specifically bind) a SARS-COV-2 spike protein. In some implementations, the single-domain monoclonal antibody includes at least a portion of the amino acid sequence set forth herein as SEQ ID NO: 5, such as one or more (such as all three) CDR sequences of RBD-1-2G, as determined by any numbering convention (such as IMGT, Kabat or Chothia). In particular examples, the CDR sequences are determined using IMGT.

In some implementations, the single-domain monoclonal antibody includes the CDR1, CDR2 and CDR3 sequences of SEQ ID NO: 5. In specific implementations, the CDR1, CDR2 and CDR3 respectively include residues 26-32, 50-58 and 97-110 of SEQ ID NO: 5. In some examples, the amino acid sequence of the single-domain monoclonal antibody is at least 80%, at least 85%, at least 90%, at least 95%, at least 96%, at least 97%, at least 98% or at least 99% identical to SEQ ID NO: 5. In some examples, the single-domain monoclonal antibody includes a framework region 1 (FR1), a FR2, a FR3 and a FR4, and the amino acid sequence of FR1 comprises SEQ ID NO: 1; the amino acid sequence of FR2 comprises SEQ ID NO: 2; the amino acid sequence of FR3 comprises SEQ ID NO: 3; and/or the amino acid sequence of FR4 comprises SEQ ID NO: 4. In particular examples, the amino acid sequence of the single-domain monoclonal antibody comprises or consists of SEQ ID NO: 5.

In some implementations, the single-domain monoclonal antibody is a humanized antibody.

In some implementations, the monoclonal antibody or antigen-binding fragment neutralizes SARS-COV-2 and/or SARS-COV-1.

In some implementations, the single-domain monoclonal antibody is conjugated to a detectable label, such as a fluorophore, an enzyme or a radioisotope.

Also provided herein are fusion proteins that include a single-domain monoclonal antibody disclosed herein and a heterologous protein. In some implementations, the heterologous protein includes an Fc protein, such as a human Fc protein or a murine Fc protein. In some examples, the Fc protein is a human Fc protein that includes a modification that increases half-life of the fusion protein. In some examples, the modification increases binding to the neonatal Fc receptor. In other implementations, the heterologous protein includes a protein tag. In some examples, the protein tag includes a His tag and/or a human influenza hemagglutinin tag. In particular examples, the amino acid sequence of the protein tag comprises or consists of SEQ ID NO: 6.

Also provided herein are bivalent antibodies that include a single-domain monoclonal antibody disclosed herein. In some implementations, the bivalent antibody includes two monomers of the single-domain monoclonal antibody fused to an Fc protein, such as a human Fc protein. In some examples, the Fc protein is a human Fc protein that includes a modification that increases half-life of the fusion protein. In some examples, the modification increases binding to the neonatal Fc receptor.

Further provided are trivalent single-chain Fv antibodies that include three monomers of a single-domain monoclonal antibody disclosed herein. In some implementations, each monomer of the trimer is separated by a flexible linker, such as (GGGGS)₃ (SEQ ID NO: 9).

Also provided herein are multi-specific antibodies, such as bispecific antibodies, that include a single-domain monoclonal antibody disclosed herein and a second antibody that specifically binds a different antigen or a different epitope of the spike protein.

Further provided herein are nucleic acid molecules that encode a single-domain monoclonal antibody, a fusion protein, a bivalent antibody, a trivalent single-chain Fv, or a bispecific antibody disclosed herein. In some implementations, the nucleic acid molecule is a cDNA sequence encoding the single-domain monoclonal antibody, fusion protein, bivalent antibody, trivalent single-chain Fv or bispecific antibody. In some implementations, the nucleic acid molecule is operably linked to a promoter, such as a heterologous promoter.

Also provided are vectors that include a nucleic acid molecule disclosed herein. In some implementations, the vector is an expression vector. In other implementations, the vector is a viral vector. Host cells that include a disclosed nucleic acid molecule or vector are further provided.

Further provided are compositions that include a pharmaceutically acceptable carrier and a single-domain monoclonal antibody, fusion protein, bivalent antibody, trivalent single-chain Fv, bispecific antibody, nucleic acid molecule or vector disclosed herein.

Also provided herein is a method of producing a single-domain monoclonal antibody that specifically binds to a SARS-COV-2 spike protein. In some implementations, the method includes expressing one or more nucleic acid molecules encoding a single-domain monoclonal antibody disclosed herein in a host cell; and purifying the single-domain monoclonal antibody.

Further provided are methods of detecting the presence of a coronavirus in a biological sample from a subject. In some implementations, the method includes contacting the biological sample with an effective amount of a single-domain monoclonal antibody disclosed herein under conditions sufficient to form an immune complex; and detecting the presence of the immune complex in the biological sample. The presence of the immune complex in the biological sample indicates the presence of the coronavirus in the sample. In some examples, the presence of the immune complex in the biological sample indicates that the subject has a SARS-COV-2 infection.

Also provided are methods of inhibiting a coronavirus infection in a subject who has or is at risk of a coronavirus infection. In some implementations, the method includes administering an effective amount of a disclosed single-domain monoclonal antibody, fusion protein, bivalent antibody, trivalent single-chain Fv, bispecific antibody, nucleic acid molecule, vector or composition to the subject. In some examples, the coronavirus is SARS-COV-2. In other examples, the coronavirus is SARS-COV-1.

Use of a disclosed single-domain monoclonal antibody, fusion protein, bivalent antibody, trivalent single-chain Fv, bispecific antibody, nucleic acid molecule, vector or composition for inhibiting a coronavirus infection in a subject or to detect the presence of a coronavirus in a biological sample is also provided. In some implementations, the coronavirus is SARS-COV-2. In other implementations, the coronavirus is SARS-COV-1.

### VI. Multispecific Antibodies

Multi-specific antibodies are recombinant proteins comprised of two or more monoclonal antibodies (such as single-domain antibodies) or antigen-binding fragments of two or more different monoclonal antibodies. For example, bispecific antibodies are comprised of two different monoclonal antibodies or antigen-binding fragments thereof. Thus, bispecific antibodies bind two different antigens (or two different epitopes) and trispecific antibodies bind three different antigens (or three different epitopes). Multi-specific antibodies can be used for treating a coronavirus infection by simultaneously targeting, for example, a coronavirus S protein (including N-terminal domain (NTD), receptor binding domain (RBD), or whole S1 or S2 subunits) and a carbohydrate (including N-glycans), envelope protein, or hemagglutinin-esterase dimer (HE). In some examples, the multi-specific antibody includes a first binding domain that targets a portion of a coronavirus S protein (such as the NTD, RBD, S1 subunit or S2 subunit) and a second binding domain that targets a different portion of the same coronavirus S protein (such as the NTD, RBD, S1 subunit or S2 subunit).

Provided herein are multi-specific, such as trispecific or bispecific, monoclonal antibodies that include an S protein-specific single-domain monoclonal antibody. In some implementations, the multi-specific monoclonal antibody further includes a monoclonal antibody that specifically binds S protein RBD, NTD, S1 subunit or S2 subunit, or a carbohydrate (such as an N-glycan), or other viral proteins (such as envelope or HE). Also provided are isolated nucleic acid molecules and vectors encoding the multi-specific antibodies, and host cells comprising the nucleic acid molecules or vectors. Multi-specific antibodies comprising a spike protein-specific antibody can be used for the treatment of a coronavirus infection. Thus, provided herein are methods of treating a subject with a coronavirus infection by administering to the subject a therapeutically effective amount of the spike protein-targeting multi-specific antibody.

Any suitable method can be used to design and produce the multi-specific antibody, such as crosslinking two or more antibodies, antigen binding fragments (such as scFvs) of the same type or of different types. Exemplary methods of making multispecific antibodies include those described in PCT Pub. No. WO 2013/163427, which is incorporated by reference herein in its entirety. Non-limiting examples of suitable crosslinkers include those that are heterobifunctional, having two distinctly reactive groups separated by an appropriate spacer (such as m-maleimidobenzoyl-N-hydroxysuccinimide ester) or homobifunctional (such as disuccinimidyl suberate).

The multi-specific antibody may have any suitable format that allows for binding to the coronavirus spike protein by the single-domain monoclonal antibody as provided herein. Bispecific single chain antibodies can be encoded by a single nucleic acid molecule. Non-limiting examples of bispecific single chain antibodies, as well as methods of constructing such antibodies are provided in U.S. Pat. Nos. 8,076,459, 8,017,748, 8,007,796, 7,919,089, 7,820,166, 7,635,472, 7,575,923, 7,435,549, 7,332,168, 7,323,440, 7,235,641, 7,229,760, 7,112,324, 6,723,538. Additional examples of bispecific single chain antibodies can be found in PCT application No. WO 99/54440; Mack et al., J. Immunol., 158 (8): 3965-3970, 1997; Mack et al., Proc. Natl. Acad. Sci. U.S.A., 92 (15): 7021-7025, 1995; Kufer et al., Cancer Immunol. Immunother., 45 (3-4): 193-197, 1997; Löffler et al., Blood, 95 (6): 2098-2103, 2000; and Brühl et al., J. Immunol., 166 (4): 2420-2426, 2001. Production of bispecific Fab-scFv (“bibody”) molecules are described, for example, in Schoonjans et al. (J. Immunol., 165 (12): 7050-7057, 2000) and Willems et al. (J. Chromatogr. B Analyt. Technol. Biomed Life Sci. 786 (1-2): 161-176, 2003). For bibodies, a scFv molecule can be fused to one of the VL-CL (L) or VH-CH1 chains, e.g., to produce a bibody one scFv is fused to the C-term of a Fab chain.

### VII. Conjugates

The single-domain monoclonal antibodies, fusion proteins, bivalent antibodies, trivalent single-chain Fvs and bispecific antibodies that specifically bind to a coronavirus spike protein, as disclosed herein, can be conjugated to an agent, such as an effector molecule or detectable marker. Both covalent and noncovalent attachment means may be used. Various effector molecules and detectable markers can be used, including (but not limited to) toxins and radioactive agents such as ¹²⁵I, ³²P, ¹⁴C, ³H and ³⁵S and other labels, target moieties, enzymes and ligands, etc. The choice of a particular effector molecule or detectable marker depends on the particular target molecule or cell, and the desired biological effect.

The procedure for attaching a detectable marker to an antibody varies according to the chemical structure of the effector. Polypeptides typically contain a variety of functional groups, such as carboxyl (—COOH), free amine (—NH₂) or sulfhydryl (—SH) groups, which are available for reaction with a suitable functional group on a polypeptide to result in the binding of the effector molecule or detectable marker. Alternatively, the antibody is derivatized to expose or attach additional reactive functional groups. The derivatization may involve attachment of any suitable linker molecule. The linker is capable of forming covalent bonds to both the antibody and to the effector molecule or detectable marker. Suitable linkers include, but are not limited to, straight or branched-chain carbon linkers, heterocyclic carbon linkers, or peptide linkers. Where the antibody and the effector molecule or detectable marker are polypeptides, the linkers may be joined to the constituent amino acids through their side chains (such as through a disulfide linkage to cysteine) or the alpha carbon, or through the amino, and/or carboxyl groups of the terminal amino acids.

In view of the large number of methods that have been reported for attaching a variety of radiodiagnostic compounds, radiotherapeutic compounds, labels (such as enzymes or fluorescent molecules), toxins, and other agents to antibodies, a suitable method for attaching a given agent to a single-domain monoclonal antibody, fusion protein, bivalent antibody, trivalent single-chain Fv or bispecific antibody can be determined.

The single-domain monoclonal antibody, fusion protein, bivalent antibody, trivalent single-chain Fv or bispecific antibody can be conjugated with a detectable marker; for example, a detectable marker capable of detection by ELISA, spectrophotometry, flow cytometry, microscopy or diagnostic imaging techniques (such as CT, computed axial tomography (CAT), MRI, magnetic resonance tomography (MTR), ultrasound, fiberoptic examination, and laparoscopic examination). Specific, non-limiting examples of detectable markers include fluorophores, chemiluminescent agents, enzymatic linkages, radioactive isotopes and heavy metals or compounds (for example super paramagnetic iron oxide nanocrystals for detection by MRI). For example, useful detectable markers include fluorescent compounds, including fluorescein, fluorescein isothiocyanate, rhodamine, 5-dimethylamine-1-napthalenesulfonyl chloride, phycoerythrin, lanthanide phosphors and the like. Bioluminescent markers are also of use, such as luciferase, green fluorescent protein (GFP), and yellow fluorescent protein (YFP). A single-domain monoclonal antibody, fusion protein, bivalent antibody, trivalent single-chain Fv or bispecific antibody can also be conjugated with enzymes that are useful for detection, such as horseradish peroxidase, β-galactosidase, luciferase, alkaline phosphatase, glucose oxidase and the like. When an antibody is conjugated with a detectable enzyme, it can be detected by adding additional reagents that the enzyme uses to produce a reaction product that can be discerned. For example, when the agent horseradish peroxidase is present, the addition of hydrogen peroxide and diaminobenzidine leads to a colored reaction product, which is visually detectable. A single-domain monoclonal antibody, fusion protein, bivalent antibody, trivalent single-chain Fv or bispecific antibody may also be conjugated with biotin, and detected through indirect measurement of avidin or streptavidin binding. It should be noted that the avidin itself can be conjugated with an enzyme or a fluorescent label.

The single-domain monoclonal antibody, fusion protein, bivalent antibody, trivalent single-chain Fv or bispecific antibody can be conjugated with a paramagnetic agent, such as gadolinium. Paramagnetic agents, such as superparamagnetic iron oxide, are also of use as labels. Antibodies can also be conjugated with lanthanides (such as europium and dysprosium) and manganese. A single-domain monoclonal antibody, fusion protein, bivalent antibody, trivalent single-chain Fv or bispecific antibody may also be labeled with a predetermined polypeptide epitope recognized by a secondary reporter (such as leucine zipper pair sequences, binding sites for secondary antibodies, metal binding domains, epitope tags).

The single-domain monoclonal antibody, fusion protein, bivalent antibody, trivalent single-chain Fv or bispecific antibody can also be conjugated with a radiolabeled amino acid, for example, for diagnostic purposes. For instance, the radiolabel may be used to detect a coronavirus by radiography, emission spectra, or other diagnostic techniques. Examples of labels for polypeptides include, but are not limited to, the following radioisotopes: ³H, ¹⁴C, ³⁵S, ⁹⁰Y, ^{99m}Tc, ¹¹¹In, ¹²⁵I, ¹³¹I. The radiolabels may be detected, for example, using photographic film or scintillation counters, fluorescent markers may be detected using a photodetector to detect emitted illumination. Enzymatic labels are typically detected by providing the enzyme with a substrate and detecting the reaction product produced by the action of the enzyme on the substrate, and colorimetric labels are detected by simply visualizing the colored label.

The average number of detectable marker moieties per antibody in a conjugate can range, for example, from 1 to 20 moieties per antibody. In some implementations, the average number of effector molecules or detectable marker moieties per antibody in a conjugate range from about 1 to about 2, from about 1 to about 3, about 1 to about 8; from about 2 to about 6; from about 3 to about 5; or from about 3 to about 4. The loading (for example, effector molecule per antibody ratio) of a conjugate may be controlled in different ways, for example, by: (i) limiting the molar excess of effector molecule-linker intermediate or linker reagent relative to antibody, (ii) limiting the conjugation reaction time or temperature, (iii) partial or limiting reducing conditions for cysteine thiol modification, and (iv) engineering by recombinant techniques the amino acid sequence of the antibody such that the number and position of cysteine residues is modified for control of the number or position of linker-effector molecule attachments.

### VIII. Antibody Variants

In some implementations, amino acid sequence variants of the single-domain monoclonal antibodies, fusion proteins, bivalent antibodies, trivalent single-chain Fvs or bispecific antibodies disclosed herein are provided. For example, it may be desirable to improve the binding affinity and/or other biological properties of the antibody, fusion protein, bivalent antibody, trivalent single-chain Fv or bispecific antibody. Amino acid sequence variants of an antibody may be prepared by introducing appropriate modifications into the nucleotide sequence encoding the single-domain monoclonal antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen binding.

In some implementations, variants having one or more amino acid substitutions are provided. Sites of interest for substitutional mutagenesis include the CDRs and the framework regions. Amino acid substitutions may be introduced into an antibody of interest and the products screened for a desired activity, e.g., retained/improved antigen binding, decreased immunogenicity, or improved ADCC or CDC.

The variants typically retain amino acid residues necessary for correct folding and will retain the charge characteristics of the residues in order to preserve the low pI and low toxicity of the molecules. Amino acid substitutions can be made in the antibody to increase yield.

In some implementations, the single-domain monoclonal antibody includes up to 10 (such as up to 1, up to 2, up to 3, up to 4, up to 5, up to 6, up to 7, up to 8, or up to 9) amino acid substitutions (such as conservative amino acid substitutions) compared to the amino acid sequence set forth as SEQ ID NO: 5.

In some implementations, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in CDRs. In some implementations of the variant antibody sequences provided above, each CDR either is unaltered, or contains no more than one, two or three amino acid substitutions. In some implementations of the variant antibody sequences provided above, only the framework residues are modified so the CDRs are unchanged.

To increase binding affinity of the single-domain monoclonal antibody, the antibody can be randomly mutated, such as within the CDR3, in a process analogous to the in vivo somatic mutation process responsible for affinity maturation of antibodies during a natural immune response. Thus in vitro affinity maturation can be accomplished by amplifying the single-domain monoclonal antibody using PCR primers complementary to the CDR3. In this process, the primers have been “spiked” with a random mixture of the four nucleotide bases at certain positions such that the resultant PCR products encode a single-domain antibody into which random mutations have been introduced into the CDR3. These randomly single-domain antibodies can be tested to determine the binding affinity for the spike protein.

In some implementations, an antibody is altered to increase or decrease the extent to which the antibody is glycosylated. Addition or deletion of glycosylation sites may be conveniently accomplished by altering the amino acid sequence such that one or more glycosylation sites is created or removed.

Where the antibody (or fusion protein) includes an Fc region, the carbohydrate attached thereto may be altered. Native antibodies produced by mammalian cells typically comprise a branched, biantennary oligosaccharide that is generally attached by an N-linkage to Asn297 of the CH₂ domain of the Fc region. See, e.g., Wright et al. Trends Biotechnol. 15 (1): 26-32, 1997. The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some implementations, modifications of the oligosaccharide in an antibody may be made in order to create antibody variants with certain improved properties.

In one implementation, variants are provided having a carbohydrate structure that lacks fucose attached (directly or indirectly) to an Fc region. For example, the amount of fucose in such an antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region; however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO 2002/031140; Okazaki et al., J. Mol. Biol., 336 (5): 1239-1249, 2004; Yamane-Ohnuki et al., Biotechnol. Bioeng. 87 (5): 614-622, 2004. Examples of cell lines capable of producing defucosylated antibodies include Lec 13 CHO cells deficient in protein fucosylation (Ripka et al., Arch. Biochem. Biophys. 249 (2): 533-545, 1986; US Pat. Appl. No. US 2003/0157108 and WO 2004/056312, especially at Example 11), and knockout cell lines, such as alpha-1,6-fucosyltransferase gene, FUT8, knockout CHO cells (see, e.g., Yamane-Ohnuki et al., Biotechnol. Bioeng., 87 (5): 614-622, 2004; Kanda et al., Biotechnol. Bioeng., 94 (4): 680-688, 2006; and WO2003/085107).

Antibody variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087; WO 1998/58964; and WO 1999/22764.

In several implementations in which the single-domain monoclonal antibody is fused to an Fc protein, the Fc region includes one or more amino acid substitutions to optimize in vivo half-life of the antibody. The serum half-life of IgG Abs is regulated by the neonatal Fc receptor (FcRn). Thus, in several implementations, the Fc region includes an amino acid substitution that increases binding to the FcRn. Non-limiting examples of such substitutions include substitutions at IgG constant regions T250Q and M428L (see, e.g., Hinton et al., J Immunol., 176 (1): 346-356, 2006); M428L and N434S (the “LS” mutation, see, e.g., Zalevsky, et al., Nature Biotechnol., 28 (2): 157-159, 2010); N434A (see, e.g., Petkova et al., Int. Immunol., 18 (12): 1759-1769, 2006); T307A, E380A, and N434A (see, e.g., Petkova et al., Int. Immunol., 18 (12): 1759-1769, 2006); and M252Y, S254T, and T256E (see, e.g., Dall'Acqua et al., J. Biol. Chem., 281 (33): 23514-23524, 2006). The disclosed single-domain antibodies can be linked to or comprise an Fc polypeptide including any of the substitutions listed above, for example, the Fc polypeptide can include the M428L and N434S (“LS”) substitutions.

In some implementations, a single-domain monoclonal antibody, fusion protein, bivalent antibody, trivalent single-chain Fv or bispecific antibody provided herein may be further modified to contain additional nonproteinaceous moieties. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone) polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in an application under defined conditions, etc.

### IX. Binding Affinity

In several implementations, the single-domain monoclonal antibody (including variants and conjugates thereof) specifically binds the coronavirus spike protein with an affinity (e.g., measured by K_{D}) of no more than 1.0×10⁻⁸ M, no more than 5.0×10⁻⁸ M, no more than 1.0×10⁻⁹ M, no more than 5.0×10⁻⁹ M, no more than 1.0×10⁻¹⁰ M, no more than 5.0×10⁻¹⁰ M, or no more than 1.0×10⁻¹¹ M. K_{D} can be measured, for example, by a radiolabeled antigen binding assay (RIA) performed with the antibody of interest and its antigen.

In one assay, K_{D} can be measured using surface plasmon resonance assays using biolayer interferometry (BLI). In other implementations, K_{D} can be measured using a BIACORE®-2000 or a BIACORE®-3000 (BIAcore, Inc., Piscataway, N.J.) at 25° C. with immobilized antigen CM5 chips at ˜10 response units (RU). Briefly, carboxymethylated dextran biosensor chips (CM5, BIACORE®, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 uM) before injection at a flow rate of 5 l/minute to achieve approximately 10 response units (RU) of coupled protein. Following the injection of antigen, 1 M ethanolamine is injected to block unreacted groups. For kinetics measurements, two-fold serial dilutions of antibody (0.78 nM to 500 nM) are injected in PBS with 0.05% polysorbate 20 (TWEEN-20™) surfactant (PBST) at 25° C. at a flow rate of approximately 25 l/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (K_{D}) is calculated as the ratio k_{off}/kₒₙ. See, e.g., Chen et al., J. Mol. Biol. 293:865-881 (1999). If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody in PBS, pH 7.2, in the presence of increasing concentrations of antigen as measured in a spectrometer, such as a stop-flow equipped spectrophotometer (Aviv Instruments) or a 8000-series SLM-AMINCO™ spectrophotometer (ThermoSpectronic) with a stirred cuvette. Affinity can also be measured by high throughput SPR using the Carterra LSA, competition radioimmunoassay, ELISA, flow cytometry, or the Octet system (Creative Biolabs), which is based on bio-layer interferometry (BLI) technology.

### X. Nucleic Acid Molecules

Nucleic acid molecules (for example, DNA, cDNA or RNA molecules) encoding the amino acid sequences of the disclosed antibodies, fusion proteins, and conjugates that specifically bind to a coronavirus spike protein, are provided. Nucleic acid molecules encoding these molecules can readily be produced using the amino acid sequences provided herein, sequences available in the art (such as framework or constant region sequences), and the genetic code. In some implementations, the nucleic acid molecules can be expressed in a host cell (such as a mammalian cell or a bacterial cell) to produce a disclosed antibody (for example, single-domain antibody, trivalent single-chain Fv or bispecific antibody), fusion protein or antibody conjugate.

The genetic code can be used to construct a variety of functionally equivalent nucleic acid sequences, such as nucleic acids that differ in their sequence but which encode the same antibody sequence, or encode a conjugate or fusion protein including the single-domain antibody sequence.

Nucleic acid molecules encoding the antibodies, fusion proteins, and conjugates that specifically bind to a coronavirus spike protein can be prepared by any suitable method including, for example, cloning of appropriate sequences or by direct chemical synthesis by standard methods. Chemical synthesis produces a single stranded oligonucleotide. This can be converted into double stranded DNA by hybridization with a complementary sequence or by polymerization with a DNA polymerase using the single strand as a template.

Exemplary nucleic acids can be prepared by cloning techniques. Examples of appropriate cloning and sequencing techniques can be found, for example, in Green and Sambrook (Molecular Cloning: A Laboratory Manual, 4^{th} ed., New York: Cold Spring Harbor Laboratory Press, 2012) and Ausubel et al. (Eds.) (Current Protocols in Molecular Biology, New York: John Wiley and Sons, including supplements).

Nucleic acids can also be prepared by amplification methods. Amplification methods include the polymerase chain reaction (PCR), the ligase chain reaction (LCR), the transcription-based amplification system (TAS), and the self-sustained sequence replication system (3SR).

The nucleic acid molecules can be expressed in a recombinantly engineered cell such as in bacterial, plant, yeast, insect and mammalian cells. The antibodies and conjugates can be expressed as individual proteins including the single-domain monoclonal antibody (linked to an effector molecule or detectable marker as needed), or can be expressed as a fusion protein. Any suitable method of expressing and purifying antibodies may be used; non-limiting examples are provided in Al-Rubeai (Ed.), Antibody Expression and Production, Dordrecht; New York: Springer, 2011).

One or more DNA sequences encoding the single-domain monoclonal antibodies, trivalent single-chain Fvs, bispecific antibodies and fusion proteins can be expressed in vitro by DNA transfer into a suitable host cell. The cell may be prokaryotic or eukaryotic. Numerous expression systems available for expression of proteins including E. coli, other bacterial hosts, yeast, and various higher eukaryotic cells, for example mammalian cells, such as the COS, CHO, HeLa and myeloma cell lines, can be used to express the disclosed antibodies and antigen binding fragments. Methods of stable transfer, meaning that the foreign DNA is continuously maintained in the host may be used.

The expression of nucleic acids encoding the single-domain monoclonal antibodies, trivalent single-chain Fvs, bispecific antibodies and fusion proteins described herein can be achieved by operably linking the DNA or cDNA to a promoter (which is either constitutive or inducible), followed by incorporation into an expression cassette. The promoter can be any promoter of interest, including a cytomegalovirus promoter. Optionally, an enhancer, such as a cytomegalovirus enhancer, is included in the construct. The cassettes can be suitable for replication and integration in either prokaryotes or eukaryotes. Typical expression cassettes contain specific sequences useful for regulation of the expression of the DNA encoding the protein. For example, the expression cassettes can include appropriate promoters, enhancers, transcription and translation terminators, initiation sequences, a start codon (i.e., ATG) in front of a protein-encoding gene, splicing signals for introns, sequences for the maintenance of the correct reading frame of that gene to permit proper translation of mRNA, and stop codons. The vector can encode a selectable marker, such as a marker encoding drug resistance (for example, ampicillin or tetracycline resistance).

To obtain high level expression of a cloned gene, it is desirable to construct expression cassettes which contain, for example, a strong promoter to direct transcription, a ribosome binding site for translational initiation (e.g., internal ribosomal binding sequences), and a transcription/translation terminator. For E. coli, this can include a promoter such as the T7, trp, lac, or lambda promoters, a ribosome binding site, and a transcription termination signal. For eukaryotic cells, the control sequences can include a promoter and/or an enhancer derived from, for example, an immunoglobulin gene, HTLV, SV40 or cytomegalovirus, and a polyadenylation sequence, and can further include splice donor and/or acceptor sequences (for example, CMV and/or HTLV splice acceptor and donor sequences). The cassettes can be transferred into the chosen host cell by any suitable method such as transformation or electroporation for E. coli and calcium phosphate treatment, electroporation or lipofection for mammalian cells. Cells transformed by the cassettes can be selected by resistance to antibiotics conferred by genes contained in the cassettes, such as the amp, gpt, neo and hyg genes.

Modifications can be made to a nucleic acid encoding an antibody described herein without diminishing its biological activity. Some modifications can be made to facilitate the cloning, expression, or incorporation of the antibody into a fusion protein. Such modifications include, for example, termination codons, sequences to create conveniently located restriction sites, and sequences to add a methionine at the amino terminus to provide an initiation site, or additional amino acids (such as poly His) to aid in purification steps.

Once expressed, the single-domain monoclonal antibodies, trivalent single-chain Fvs, bispecific antibodies and fusion proteins can be purified according to standard procedures in the art, including ammonium sulfate precipitation, affinity columns, column chromatography, and the like (see, generally, Simpson et al. (Eds.), Basic methods in Protein Purification and Analysis: A Laboratory Manual, New York: Cold Spring Harbor Laboratory Press, 2009). The single-domain monoclonal antibodies, trivalent single-chain Fvs, bispecific antibodies and fusion proteins need not be 100% pure. Once purified, partially or to homogeneity as desired, if to be used prophylactically, the antibodies should be substantially free of endotoxin.

Methods for expression of antibodies and/or refolding to an appropriate active form, from mammalian cells, and bacteria such as E. coli have been described and are applicable to the antibodies disclosed herein. See, e.g., Greenfield (Ed.), Antibodies: A Laboratory Manual, 2^{nd} ed. New York: Cold Spring Harbor Laboratory Press, 2014, Simpson et al. (Eds.), Basic methods in Protein Purification and Analysis: A Laboratory Manual, New York: Cold Spring Harbor Laboratory Press, 2009, and Ward et al., Nature 341 (6242): 544-546, 1989.

### XI. Antibody Compositions and Methods of Use

The single-domain monoclonal antibodies, trivalent single-chain Fvs, multi-specific (such as bispecific) antibodies, fusion proteins, compositions, nucleic acid molecules and vectors disclosed herein can be used, for example, in methods for the detection/diagnosis of coronavirus (such as SARS-COV-2) infection and in the prophylaxis and treatment of a coronavirus (such as SARS-COV-2) infection.

### A. Methods of Detection and Diagnosis

Methods are provided for the detection of the presence of a coronavirus spike protein in vitro or in vivo. In one example, the presence of a coronavirus spike protein is detected in a biological sample from a subject and can be used to identify a subject with an infection. The sample can be any sample, including, but not limited to, tissue from biopsies, autopsies and pathology specimens. Biological samples also include sections of tissues, for example, frozen sections taken for histological purposes. Biological samples further include body fluids, such as blood, serum, plasma, sputum, spinal fluid or urine. The method of detection can include contacting a cell or sample, with a single-domain domain monoclonal antibody, fusion protein, bivalent antibody, trivalent single-chain Fv or bispecific antibody that specifically binds to a coronavirus spike protein, or conjugate thereof (e.g., a conjugate including a detectable marker) under conditions sufficient to form an immune complex, and detecting the immune complex (e.g., by detecting a detectable marker conjugated to the antibody or antigen binding fragment).

In some examples, the presence of a coronavirus spike protein is detected in a biological sample from a subject and can be used to identify a subject with a coronavirus infection. The sample can be any sample, including, but not limited to, sputum, saliva, mucus, nasal wash, nasopharyngeal samples, oropharyngeal samples, peripheral blood, tissue, cells, urine, tissue biopsy, fine needle aspirate, surgical specimen, feces, cerebral spinal fluid (CSF), and bronchoalveolar lavage (BAL) fluid. Biological samples also include sections of tissues, for example, frozen sections taken for histological purposes. The method of detection can include contacting a cell or sample, with an antibody or antibody conjugate (e.g., a conjugate including a detectable marker) that specifically binds to a coronavirus spike protein, under conditions sufficient to form an immune complex, and detecting the immune complex (e.g., by detecting a detectable marker conjugated to the antibody.

In one implementation, the antibody is directly labeled with a detectable marker. In another implementation, the antibody that binds the coronavirus spike protein (the primary antibody) is unlabeled and a secondary antibody or other molecule that can bind the primary antibody is utilized for detection. The secondary antibody that is chosen is able to specifically bind the specific species and class of the first antibody. For example, if the first antibody is a human IgG, then the secondary antibody may be an anti-human-IgG. Other molecules that can bind to antibodies include, without limitation, Protein A and Protein G, both of which are available commercially.

Suitable labels for the antibody or secondary antibody include various enzymes, prosthetic groups, fluorescent materials, luminescent materials, magnetic agents and radioactive materials. Non-limiting examples of suitable enzymes include horseradish peroxidase, alkaline phosphatase, beta-galactosidase, or acetylcholinesterase. Non-limiting examples of suitable prosthetic group complexes include streptavidin/biotin and avidin/biotin. Non-limiting examples of suitable fluorescent materials include umbelliferone, fluorescein, fluorescein isothiocyanate, rhodamine, dichlorotriazinylamine fluorescein, dansyl chloride or phycoerythrin. A non-limiting exemplary luminescent material is luminol; a non-limiting exemplary a magnetic agent is gadolinium, and non-limiting exemplary radioactive labels include ¹²⁵I, ¹³¹I, ³⁵S or ³H.

In an alternative implementation, spike protein can be assayed in a biological sample by a competition immunoassay utilizing spike protein standards labeled with a detectable substance and an unlabeled antibody that specifically binds spike protein. In this assay, the biological sample, the labeled spike protein standards and the antibody that specifically bind spike protein are combined and the amount of labeled spike protein standard bound to the unlabeled antibody is determined. The amount of spike protein in the biological sample is inversely proportional to the amount of labeled spike protein standard bound to the antibody that specifically binds spike protein.

The immunoassays and methods disclosed herein can be used for a number of purposes. In one implementation, the antibody that specifically binds coronavirus spike protein may be used to detect the production of spike protein in cells in cell culture. In another implementation, the antibody can be used to detect the amount of spike protein in a biological sample, such as a sample obtained from a subject having or suspected or having a coronavirus infection.

In one implementation, a kit is provided for detecting coronavirus spike protein in a biological sample, such as a nasopharyngeal, oropharyngeal, sputum, saliva, or blood sample. Kits for detecting a coronavirus infection will typically include a single-domain monoclonal antibody that specifically binds coronavirus spike protein, such as any of the antibodies or conjugates disclosed herein. In a further implementation, the antibody is labeled (for example, with a fluorescent, radioactive, or an enzymatic label).

In one implementation, a kit includes instructional materials disclosing means of use of an antibody that binds coronavirus spike protein. The instructional materials may be written, in an electronic form (such as a computer diskette or compact disk) or may be visual (such as video files). The kits may also include additional components to facilitate the particular application for which the kit is designed. Thus, for example, the kit may additionally contain means of detecting a label (such as enzyme substrates for enzymatic labels, filter sets to detect fluorescent labels, appropriate secondary labels such as a secondary antibody, or the like). The kits may additionally include buffers and other reagents routinely used for the practice of a particular method. Such kits and appropriate contents are well known to those of skill in the art.

In one implementation, the diagnostic kit includes an immunoassay. Although the details of the immunoassays may vary with the particular format employed, the method of detecting spike protein in a biological sample generally includes the steps of contacting the biological sample with an antibody which specifically reacts, under immunologically reactive conditions, to coronavirus spike protein. The antibody is allowed to specifically bind under immunologically reactive conditions to form an immune complex, and the presence of the immune complex (bound antibody) is detected directly or indirectly.

The antibodies disclosed herein can also be utilized in immunoassays, such as, but not limited to radioimmunoassays (RIAs), ELISA, lateral flow assay (LFA), or immunohistochemical assays. The antibodies can also be used for fluorescence activated cell sorting (FACS), such as for identifying/detecting virus-infected cells. FACS employs a plurality of color channels, low angle and obtuse light-scattering detection channels, and impedance channels, among other more sophisticated levels of detection, to separate or sort cells (see U.S. Pat. No. 5,061,620). Any of the monoclonal antibodies or antigen-binding fragments that bind spike protein, as disclosed herein, can be used in these assays. Thus, the antibodies can be used in a conventional immunoassay, including, without limitation, ELISA, RIA, LFA, FACS, tissue immunohistochemistry, Western blot or immunoprecipitation. The disclosed antibodies can also be used in nanotechnology methods, such as microfluidic immunoassays, which can be used to capture coronavirus (such as SARS-COV-2), or exosomes containing coronavirus. Suitable samples for use with a microfluidic immunoassay or other nanotechnology method, include but are not limited to, saliva, blood, and fecal samples. Microfluidic immunoassays are described in U.S. Patent Application No. 2017/0370921, 2018/0036727, 2018/0149647, 2018/0031549, 2015/0158026 and 2015/0198593; and in Lin et al., JALA June 2010, pages 254-274; Lin et al., Anal Chem 92:9454-9458, 2020; and Herr et al., Proc Natl Acad Sci USA 104 (13): 5268-5273, 2007, all of which are herein incorporated by reference).

In some implementations, the disclosed single-domain monoclonal antibodies, fusion proteins, bivalent antibodies, trivalent single-chain Fvs or bispecific antibodies are used to test vaccines. For example, to test if a vaccine composition including a coronavirus spike protein or fragment thereof assumes a conformation including the epitope of a disclosed antibody. Thus, provided herein is a method for testing a vaccine, wherein the method includes contacting a sample containing the vaccine, such as a coronavirus spike protein immunogen, with a disclosed single-domain monoclonal antibody, fusion protein, bivalent antibody, trivalent single-chain Fv or bispecific antibody, under conditions sufficient for formation of an immune complex, and detecting the immune complex, to detect the vaccine including the epitope of interest in the sample. In one example, the detection of the immune complex in the sample indicates that vaccine component, such as the immunogen assumes a conformation capable of binding the antibody or antigen binding fragment.

### B. Methods of Treating or Inhibiting a Coronavirus Infection

Methods are disclosed herein for treating or inhibiting a coronavirus infection in a subject, such as a SARS-COV-2 infection. The methods include administering to the subject an effective amount (such as an amount effective to inhibit the infection in the subject) of a disclosed single-domain monoclonal antibody, fusion protein, bivalent antibody, trivalent single-chain Fv, bispecific antibody, nucleic acid molecule, vector or composition, to a subject at risk of a coronavirus infection or having the coronavirus infection. The methods can be used pre-exposure or post-exposure.

The infection does not need to be completely eliminated or inhibited for the method to be effective. For example, the method can decrease the infection by a desired amount, for example by at least 10%, at least 20%, at least 50%, at least 60%, at least 70%, at least 80%, at least 90%, at least 95%, at least 98%, or even at least 100% (elimination or prevention of detectable coronavirus infection) as compared to the coronavirus infection in the absence of the treatment. In some implementations, the subject can also be treated with an effective amount of an additional agent, such as an anti-viral agent.

In some implementations, administration of an effective amount of a disclosed single-domain monoclonal antibody, fusion protein, bivalent antibody, trivalent single-chain Fv, bispecific antibody, nucleic acid molecule, vector or composition inhibits the establishment of an infection and/or subsequent disease progression in a subject, which can encompass any statistically significant reduction in activity (for example, virus replication) or symptoms of the coronavirus infection in the subject (such as fever or cough).

Methods are disclosed herein for the inhibition of coronavirus replication in a subject. The methods include administering to the subject an effective amount (that is, an amount effective to inhibit replication in the subject) of a disclosed single-domain monoclonal antibody, fusion protein, bivalent antibody, trivalent single-chain Fv, bispecific antibody, nucleic acid molecule, vector or composition to a subject at risk of a coronavirus infection or having a coronavirus infection. The methods can be used pre-exposure or post-exposure.

Methods are disclosed for treating a coronavirus infection in a subject. Methods are also disclosed for preventing a coronavirus infection in a subject. These methods include administering one or more of the disclosed single-domain monoclonal antibodies, fusion proteins, bivalent antibodies, trivalent single-chain Fvs, bispecific antibodies, nucleic acid molecules, vectors or compositions.

Antibodies and fusion proteins can be administered, for example, by intravenous infusion. Doses of the single-domain monoclonal antibody, fusion protein, bivalent antibody, trivalent single-chain Fv or bispecific antibody vary, but generally range between about 0.5 mg/kg to about 50 mg/kg, such as a dose of about 1 mg/kg, about 5 mg/kg, about 10 mg/kg, about 20 mg/kg, about 30 mg/kg, about 40 mg/kg, or about 50 mg/kg. In some implementations, the dose of the single-domain monoclonal antibody, fusion protein, bivalent antibody, trivalent single-chain Fv or bispecific antibody can be from about 0.5 mg/kg to about 5 mg/kg, such as a dose of about 1 mg/kg, about 2 mg/kg, about 3 mg/kg, about 4 mg/kg or about 5 mg/kg. The single-domain monoclonal antibody, fusion protein, bivalent antibody, trivalent single-chain Fv or bispecific antibody is administered according to a dosing schedule determined by a medical practitioner. In some examples, the single-domain monoclonal antibody, fusion protein, bivalent antibody, trivalent single-chain Fv or bispecific antibody is administered weekly, every two weeks, every three weeks or every four weeks.

In some implementations, the method of inhibiting the infection in a subject further comprises administration of one or more additional agents to the subject. Additional agents of interest include, but are not limited to, anti-viral agents such as hydroxychloroquine, arbidol, remdesivir, favipiravir, baricitinib, lopinavir/ritonavir, Zinc ions, and interferon beta-1b, or their combinations.

In some implementations, the method includes administration of a first antibody that specifically binds to a coronavirus spike protein as disclosed herein and a second antibody that also specifically binds to a coronavirus protein, such as a different epitope of the coronavirus protein.

In some implementations, a subject is administered DNA or RNA encoding a disclosed single-domain monoclonal antibody, fusion protein, bivalent antibody, trivalent single-chain Fv or bispecific antibody to provide in vivo antibody production, for example using the cellular machinery of the subject. Any suitable method of nucleic acid administration may be used; non-limiting examples are provided in U.S. Pat. Nos. 5,643,578, 5,593,972 and 5,817,637. U.S. Pat. No. 5,880,103 describes several methods of delivery of nucleic acids encoding proteins to an organism. One approach to administration of nucleic acids is direct administration with plasmid DNA, such as with a mammalian expression plasmid. The nucleotide sequence encoding the disclosed single-domain monoclonal antibody, fusion protein, bivalent antibody, trivalent single-chain Fv or bispecific antibody can be placed under the control of a promoter to increase expression. The methods include liposomal delivery of the nucleic acids. Such methods can be applied to the production of a single-domain monoclonal antibody, fusion protein, bivalent antibody, trivalent single-chain Fv or bispecific antibody. In some implementations, a disclosed single-domain monoclonal antibody, fusion protein, bivalent antibody, trivalent single-chain Fv or bispecific antibody is expressed in a subject using the pVRC8400 vector (described in Barouch et al., J. Virol., 79 (14), 8828-8834, 2005, which is incorporated by reference herein).

In several implementations, a subject (such as a human subject at risk of a coronavirus infection or having a coronavirus infection) can be administered an effective amount of a viral vector that comprises one or more nucleic acid molecules encoding a disclosed single-domain monoclonal antibody, fusion protein, bivalent antibody, trivalent single-chain Fv or bispecific antibody. The viral vector is designed for expression of the nucleic acid molecules encoding a disclosed single-domain monoclonal antibody, fusion protein, bivalent antibody, trivalent single-chain Fv or bispecific antibody, and administration of the effective amount of the viral vector to the subject leads to expression of an effective amount of the single-domain monoclonal antibody, fusion protein, bivalent antibody, trivalent single-chain Fv or bispecific antibody in the subject. Non-limiting examples of viral vectors that can be used to express a disclosed single-domain monoclonal antibody, fusion protein, bivalent antibody, trivalent single-chain Fv or bispecific antibody in a subject include those provided in Johnson et al., Nat. Med., 15 (8): 901-906, 2009 and Gardner et al., Nature, 519 (7541): 87-91, 2015, each of which is incorporated by reference herein in its entirety.

In one implementation, a nucleic acid encoding a disclosed single-domain monoclonal antibody, fusion protein, bivalent antibody, trivalent single-chain Fv or bispecific antibody is introduced directly into tissue. For example, the nucleic acid can be loaded onto gold microspheres by standard methods and introduced into the skin by a device such as Bio-Rad's HELIOS™ Gene Gun. The nucleic acids can be “naked,” consisting of plasmids under control of a strong promoter.

Typically, the DNA is injected into muscle, although it can also be injected directly into other sites. Dosages for injection are usually around 0.5 μg/kg to about 50 mg/kg, and typically are about 0.005 mg/kg to about 5 mg/kg (see, e.g., U.S. Pat. No. 5,589,466).

Single or multiple administrations of a composition including a disclosed single-domain monoclonal antibody, fusion protein, bivalent antibody, trivalent single-chain Fv or bispecific antibody, or nucleic acid molecule encoding such molecules, can be administered depending on the dosage and frequency as required and tolerated by the patient. The dosage can be administered once, but may be applied periodically until either a desired result is achieved or until side effects warrant discontinuation of therapy. Generally, the dose is sufficient to inhibit a coronavirus infection without producing unacceptable toxicity to the patient.

Data obtained from cell culture assays and animal studies can be used to formulate a range of dosage for use in humans. The dosage normally lies within a range of circulating concentrations that include the ED₅₀, with little or minimal toxicity. The dosage can vary within this range depending upon the dosage form employed and the route of administration utilized. The effective dose can be determined from cell culture assays and animal studies.

The coronavirus spike protein-specific single-domain monoclonal antibody, fusion protein, bivalent antibody, trivalent single-chain Fv or bispecific antibody or nucleic acid molecule encoding such molecules, or a composition including such molecules, can be administered to subjects in various ways, including local and systemic administration, such as, e.g., by injection subcutaneously, intravenously, intra-arterially, intraperitoneally, intramuscularly, intradermally, or intrathecally. In an implementation, the single-domain monoclonal antibody, fusion protein, bivalent antibody, trivalent single-chain Fv or bispecific antibody or nucleic acid molecule encoding such molecules, or a composition including such molecules, is administered by a single subcutaneous, intravenous, intra-arterial, intraperitoneal, intramuscular, intradermal or intrathecal injection once a day. The single-domain monoclonal antibody, fusion protein, bivalent antibody, trivalent single-chain Fv or bispecific antibody, or nucleic acid molecule encoding such molecules, or a composition including such molecules, can also be administered by direct injection at or near the site of disease. A further method of administration is by osmotic pump (e.g., an Alzet pump) or mini-pump (e.g., an Alzet mini-osmotic pump), which allows for controlled, continuous and/or slow-release delivery of the single-domain monoclonal antibody, fusion protein, bivalent antibody, trivalent single-chain Fv or bispecific antibody, or nucleic acid molecule encoding such molecules, or a composition including such molecules, over a pre-determined period. The osmotic pump or mini-pump can be implanted subcutaneously, or near a target site.

### C. Compositions

Compositions are provided that include one or more of the coronavirus spike protein-specific single-domain monoclonal antibody, fusion protein, bivalent antibody, trivalent single-chain Fv or bispecific antibody, or nucleic acid molecule encoding such molecules, that are disclosed herein in a pharmaceutically acceptable carrier. In some implementations, the composition comprises the RBD-1-2G antibody disclosed herein, or a conjugate there. In some implementations, the composition comprises two, three, four or more antibodies (including RBD-1-2G) that specifically bind a coronavirus spike protein. The compositions are useful, for example, for example, for the inhibition or detection of a coronavirus infection, such as a SARS-COV-2 infection.

The compositions can be prepared in unit dosage forms, such as in a kit, for administration to a subject. The amount and timing of administration are at the discretion of the administering physician to achieve the desired purposes. The single-domain monoclonal antibody, fusion protein, bivalent antibody, trivalent single-chain Fv or bispecific antibody, or nucleic acid molecule encoding such molecules can be formulated for systemic or local administration. In one example, the single-domain monoclonal antibody, fusion protein, bivalent antibody, trivalent single-chain Fv or bispecific antibody, or nucleic acid molecule encoding such molecules, is formulated for parenteral administration, such as intravenous administration.

In some implementations, the single-domain monoclonal antibody, fusion protein, bivalent antibody, trivalent single-chain Fv or bispecific antibody in the composition is at least 70% (such as at least 75%, at least 80%, at least 85%, at least 90%, at least 95%, at least 96%, at least 97%, at least 98% or at least 99%) pure. In some implementations, the composition contains less than 10% (such as less than 5%, less than 4%, less than 3%, less than 2%, less than 1%, less than 0.5%, or even less) of macromolecular contaminants, such as other mammalian (e.g., human) proteins.

The compositions for administration can include a solution of the single-domain monoclonal antibody, fusion protein, bivalent antibody, trivalent single-chain Fv or bispecific antibody, or nucleic acid molecule encoding such molecules, dissolved in a pharmaceutically acceptable carrier, such as an aqueous carrier. A variety of aqueous carriers can be used, for example, buffered saline and the like. These solutions are sterile and generally free of undesirable matter. These compositions may be sterilized by any suitable technique. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of antibody in these formulations can vary widely, and will be selected primarily based on fluid volumes, viscosities, body weight and the like in accordance with the particular mode of administration selected and the subject's needs.

A typical composition for intravenous administration comprises about 0.01 to about 30 mg/kg of single-domain monoclonal antibody, fusion protein, bivalent antibody, trivalent single-chain Fv or bispecific antibody per subject per day (or the corresponding dose of a conjugate including the antibody). Any suitable method may be used for preparing administrable compositions; non-limiting examples are provided in such publications as Remington: The Science and Practice of Pharmacy, 22^{nd} ed., London, UK: Pharmaceutical Press, 2013. In some implementations, the composition can be a liquid formulation including one or more single-domain monoclonal antibodies, fusion proteins, bivalent antibodies, trivalent single-chain Fvs, or bispecific antibodies, in a concentration range from about 0.1 mg/ml to about 20 mg/ml, or from about 0.5 mg/ml to about 20 mg/ml, or from about 1 mg/ml to about 20 mg/ml, or from about 0.1 mg/ml to about 10 mg/ml, or from about 0.5 mg/ml to about 10 mg/ml, or from about 1 mg/ml to about 10 mg/ml.

A single-domain monoclonal antibody, fusion protein, bivalent antibody, trivalent single-chain Fv or bispecific antibody, or a nucleic acid encoding such molecules, can be provided in lyophilized form and rehydrated with sterile water before administration, although they are also provided in sterile solutions of known concentration. A solution including the single-domain monoclonal antibody, fusion protein, bivalent antibody, trivalent single-chain Fv or bispecific antibody, or a nucleic acid encoding such molecules, can then be added to an infusion bag containing 0.9% sodium chloride, USP, and typically administered at a dosage of from 0.5 to 15 mg/kg of body weight. Considerable experience is available in the art in the administration of antibody drugs, which have been marketed in the U.S. since the approval of Rituximab in 1997. Single-domain monoclonal antibodies, fusion proteins, bivalent antibodies, trivalent single-chain Fvs or bispecific antibodies, or a nucleic acid encoding such molecules, can be administered by slow infusion, rather than in an intravenous push or bolus. In one example, a higher loading dose is administered, with subsequent, maintenance doses being administered at a lower level. For example, an initial loading dose of 4 mg/kg may be infused over a period of some 90 minutes, followed by weekly maintenance doses for 4-8 weeks of 2 mg/kg infused over a 30-minute period if the previous dose was well tolerated.

Controlled-release parenteral formulations can be made as implants, oily injections, or as particulate systems. For a broad overview of protein delivery systems see, Banga, Therapeutic Peptides and Proteins: Formulation, Processing, and Delivery Systems, Lancaster, PA: Technomic Publishing Company, Inc., 1995. Particulate systems include microspheres, microparticles, microcapsules, nanocapsules, nanospheres, and nanoparticles. Microcapsules contain the active protein agent, such as a cytotoxin or a drug, as a central core. In microspheres, the active protein agent is dispersed throughout the particle. Particles, microspheres, and microcapsules smaller than about 1 μm are generally referred to as nanoparticles, nanospheres, and nanocapsules, respectively. Capillaries have a diameter of approximately 5 μm so that only nanoparticles are administered intravenously. Microparticles are typically around 100 μm in diameter and are administered subcutaneously or intramuscularly. See, for example, Kreuter, Colloidal Drug Delivery Systems, J. Kreuter (Ed.), New York, NY: Marcel Dekker, Inc., pp. 219-342, 1994; and Tice and Tabibi, Treatise on Controlled Drug Delivery: Fundamentals, Optimization, Applications, A. Kydonieus (Ed.), New York, NY: Marcel Dekker, Inc., pp. 315-339, 1992.

Polymers can be used for ion-controlled release of the compositions disclosed herein. Any suitable polymer may be used, such as a degradable or nondegradable polymeric matrix designed for use in controlled drug delivery. Alternatively, hydroxyapatite has been used as a microcarrier for controlled release of proteins. In yet another aspect, liposomes are used for controlled release as well as drug targeting of the lipid-capsulated drug.

The following examples are provided to illustrate certain particular features and/or implementations. These examples should not be construed to limit the disclosure to the particular features or implementations described.

### EXAMPLES

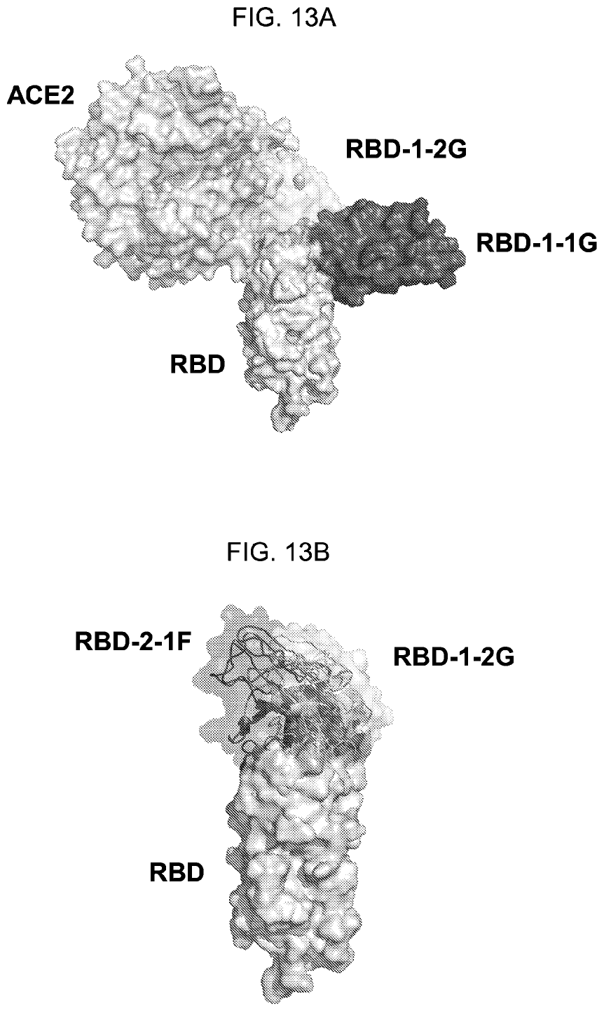

The Examples below describe the isolation and characterization of SARS-COV-2 neutralizing single-domain antibodies from a humanized phage library. These nanobodies bind the SARS-COV-2 spike RBD with single-digit nM to uM affinity, and are capable of neutralizing S-protein pseudotyped and authentic viruses in mammalian cell models of SARS-COV-2 infection. Nanobody RBD-1-2G showed the best overall potency and improved viral neutralization when incorporated into bivalent and trivalent modalities. RBD-1-2G binds an epitope on the top of the RBD that overlaps with the binding site for ACE2 (FIG. 13A). The high activity observed for RBD-1-2G was not only attributed to its high affinity, but also due to the ability of this nanobody to bind the RBD in a wide range of conformations from the “up” to the “down” states. Cryo-EM studies have revealed two prevalent states of S trimer: three RBD domains in “down” conformation which may indicate a conformational immune escape mechanism of action (Walls et al., Cell 181, 281-292 e286, 2020), or only one RBD in the “up” conformation corresponding to the ACE2-accessible state (Walls et al., Cell 181, 281-292 e286, 2020; Wrapp et al., Cell 181, 1004-1015, 2020). Some nanobodies could not be imaged in multiple RBD conformations, which would suggest that conformation switching to a protected, three RBD down conformation may sterically restrict binding. The ability of RBD-1-2G to bind in both the “up” and “half down” states allows three nanobodies to bind to a single spike trimer regardless of the RBD conformation state.

### Example 1: Methods

This example describes the materials and experimental procedures for the studies described in Examples 2-7.

### Cell Lines

Cell lines were obtained from ATCC (HEK293 cells). ACE2-GFP HEK293T (CB-97100-203) cells were purchased from Codex Biosolutions. Expi293F cells with stable expression of human ACE2 (HEK293-ACE2) were custom produced by Codex Biosolutions (Gaithersburg, MD) (Huang et al., Nat Biotechnol 39, 747-753, 2021). All cell lines were routinely tested for mycoplasma and found to be mycoplasma-free.

### Antibody Library Construction

The DNA library of nanobodies was constructed by three-step overlap-extension PCR (OE-PCR). CDR mutagenesis strategies were designed based on analysis of nanobody and 892 human heavy chain of choice of amino acids in CDR3 from PDB since 2019 or IDT trimer 19 mix (McMahon et al., Nat Struct Mol Biol 25, 289-296, 2018). Codon balanced libraries as shown in FIG. 1A were synthesized by Glen Research and Keck Biotechnology Resource Laboratory. Primers were used to amplify the backbone of a humanized nanobody backbone of caplacizumab (U.S. Pat. No. 10,919,980) to generate a library of nanobody using vector pComb3XLambda (Addgene, Plasmid #63892). Following ligation, the DNA library was transformed into TG1 electrocompetent cells (Lucigen) with an efficiency of approximately 10¹⁰ CFU per library before amplification. To amplify the library, the phagemid library was added into 1 L 2TYAG medium (2% glucose, 100 μg/ml ampicillin) and shaken at 30° C. overnight. The overnight culture of library phagemids was collected through centrifugation at 3,500 rpm for 45 minutes, and resuspended in 10 ml 50% 2TY/50% glycerol (v/v) to freeze down aliquots for making phage library. Each aliquot of 1.5 ml phagemids glycerol stock contained approximately 5× cell number of the library size. One aliquot was shaken at 37° C., 250 rpm until OD600 reached 0.9-1.0 in 1 L of 2TYAG medium, then 250 μl of M13KO7 helper phage (5×10¹²/ml) was added to each 1 L culture solution for a final concentration of 5×10⁹ and used to infect cells at 37° C. for 60 minutes. The culture was centrifuged at 3500 rpm for 10 minutes and the pellet was resuspended in 1 L 2TYKA (50 μg/ml kanamycin, 100 g/ml ampicillin). The culture was shaken overnight at 30° C. to produce phage particles. The supernatant containing the phage library was precipitated in 30% volume of PEG/NaCl overnight under refrigeration and dissolved in a total of 12 ml PBS to reach a concentration at 1014/ml in PBS/glycerol stock (UV-Vis). To recapitulate different lengths of CDRs observed in nanobody V_{H}H domains and human antibody heavy chains, different libraries containing different lengths of CDRs were made individually.

Isolation of RBD Binders from Phage Library

On Day 1:500 μl of 10 μg/ml RBD-mFc was coated in a 5 ml immunotube (NUNC, 444202) overnight at 4° C. Day 2: A series of 10 libraries were mixed and used for phage panning (250 μL in EP tube). The libraries and the coated immunotube (pre-washed three times with PBS) were blocked with 10% (w/v) skim milk for 1.5 hours at room temperature (RT). The immunotube was rinsed with PBS three times, then 0.5 ml pre-blocked phage solution was added before shaking at RT (300 rpm) for 1.5 hours. The immunotube was rinsed with PBST ten times, then PBS ten times to remove the unbound phage. Finally, RBD-bound phage were eluted by incubating with 0.5 ml freshly made 100 mM triethylamine at RT for 15 minutes. The eluted phage was further neutralized by 250 μl 1 M Tris-HCL buffer (pH 7.4). The neutralized phage (375 L) was added to 3 mL TG1 (OD=0.6-0.8) to infect at 37° C. shaking for 60 minutes. The infected TG1 cells were collected at 3500 rpm for 10 minutes and spread in vented QTray bioassay trays and titrations were plated in 2XYT agar plates with 2% glucose, 100 μg/ml ampicillin (Teknova, Y4295 and Y4204) and incubated overnight at 30° C. On Day 3: All colonies were scraped off the plates with 5 ml 2TY media, and 200 μL was removed and grown in 25 mL 2TYAG until an OD600 of 0.5-0.8 was reached. At this point, helper phage (final concentration of 5×10⁹ per ml) was added and incubated with shaking at 37° C. for 60 minutes before changing the medium to 2TYKA for phage production at 30° C. with shaking overnight.

### Antibody and Spike Protein Expression and Purification

Nanobody-Fc format was produced through CRO company using HEK293 transient expression system (Sino Biological). Spike protein was expressed and purified using the method published previously (Esposito et al., Protein Expres Purif 174, 105686, 2020). Nanobodies were expressed in Vibrio natriegens in auto-induction media (ZYM-20052) as outlined in Taylor et al. (Methods Mol Biol 1586, 65-82, 2017), with minor modifications for Vibrio. Specifically, media was amended with 1.5% (w/v) NaCl or Instant Ocean (Aquarium Systems), no lactose was added, IPTG induction began at an OD600 of 4-5, induction temperature was 30° C., and cells were harvested ˜6-8 hours after induction and frozen at −80° C. Frozen cell pellets were thawed and resuspended in 10 ml of PBS per 1000 optical density (OD600) units. Homogenized cells were lysed by passing thrice through a microfluidizer at 9,000 psi. Lysates were clarified by centrifugation at 7,900×g for 30 minutes at 4° C. Clarified lysates were filtered through 0.45 μM Whatman PES syringe filters. Nanobodies were purified using NGC medium-pressure chromatography systems (BioRad, Inc.). Clarified lysates were thawed, adjusted to 35 mM imidazole, and loaded at 3 ml/minute onto HiTrap HP IMAC columns equilibrated in IMAC equilibration buffer (EB) of PBS, pH 7.4, 35 mM imidazole, and 1:1000 protease inhibitor cocktail. The columns were washed to baseline with EB and proteins eluted with a 20 column-volume (CV) gradient from 35 mM to 500 mM imidazole in EB. Elution fractions were analyzed by SDS-PAGE and Coomassie-staining. Positive fractions were pooled and further purified by size exclusion chromatography (SEC) using appropriately sized columns packed with Superdex 75 resin and equilibrated with PBS. After analysis of fractions from the SEC by SDS-PAGE and Coomassie-staining, appropriate fractions were pooled, concentrated in 10K MWCO Amicon Ultra centrifugation units, and snap frozen in liquid nitrogen (Taylor et al., Methods Mol Biol 1586, 65-82, 2017).

### Nanobody Trimer Expression and Purification

Protein expression constructs for production of trimeric nanobodies in insect cells were generated by synthesis of optimized DNA templates (ATUM, Inc.). Trimeric nanobody proteins were preceded by a honeybee mellitin (HBM) leader sequence for secretion and followed by a C-terminal His6 tag. DNA was flanked by attB1 and attB2 Gateway recombinational cloning sites and optimized for insect cell expression using ATUM's algorithms. Templates were recombined using Gateway BP recombination (ThermoFisher) to generate entry clones, and subcloned into pDest-8, a pFastbac style baculovirus expression vector which utilizes the polyhedrin promoter to generate recombinant protein. Bacmid DNA was produced using the standard instructions for the Bac-to-Bac kit (ThermoFisher) and used to transfect Sf9 cells to generate baculovirus supernatants. Expression was performed in Tni-FNL cells at 7×10⁵ cells/ml and set to shake at 27° C. for 24 hours prior to infection. After 24 hours, the cells were counted and infected with baculovirus expressing the nanobody of interest at a MOI of 3, and cultures were shaken at 27° C. for 72 hours. After 72 hours, the cells were centrifuged at 1700×g for 15 minutes and the supernatant was collected. The supernatant was then dialyzed against 1×PBS, pH 7.4 overnight at 4° C. to remove any additives that might strip the nickel column. The dialyzed supernatant was amended with 2 M imidazole to a final concentration of 25 mM imidazole and was then loaded at 5 ml/minute onto a HiTrap HP IMAC column equilibrated in IMAC EB of 1×PBS, pH 7.4 25 mM imidazole. The column was washed to baseline with EB and proteins were eluted with a 20 CV gradient from 25 mM-500 mM imidazole in EB. Elution fractions were analyzed by SDS-PAGE and Coomassie-staining. Positive fractions were pooled and further purified by SEC using appropriately sized columns packed with Superdex S-75 resin and equilibrated with 1×PBS, pH 7.4. After analysis of fractions from SEC by SDS-PAGE and Coomassie-staining, appropriate fractions were pooled, concentrated in a 10K MWCO Amicon ultra centrifugation unit and snap frozen in liquid nitrogen. All purification processes were carried out using BioRad NGC Quest FPLC systems.

### Affinity Determination Using Octet (BLI, Biolayer Interferometry)

Affinity determination of VHHs were carried out using RBD-mFc or S1-hFc recombinant proteins (Sino Biological, 40592-V05H and 4059I-V02H, respectively) as analytes. VHHs were loaded at 10 μg/ml in kinetics buffer (PBS with 0.1% protease-free BSA and 0.02% Tween-20 in PBS) onto Ni-NTA biosensors (Molecular Devices, ForteBio). Biosensors were hydrated for 10 minutes in water, then the plate was preincubated for 10 minutes at 30° C. before the experiment started. Experimental parameters were baseline=1 minute, loading=5 minutes, baseline 2=3 minutes, association=5 minutes, dissociation=10 minutes. Association of RBD-mFc or S1-hFc was performed at 200, 100, 50 and 0 nM for all conditions, with 25 and 12.5 nM also included for the RBD-1-2G, RBD-2-1F and RBD-1-1E nanobodies. For data analysis, the 0 nM analyte with nanobody loaded was subtracted from corresponding nanobody loaded sensors. Alignment of curves to the last 5 seconds of baseline 2, inter-step correction aligned to dissociation and Stavitzky-Golay filtering was used. Data was processed, then a 1:2 bivalent analyte model with Global Fit was used for affinity calculations (Data Analysis HT 11.1).

### Affinity Determination RBD-1-2G Multimeric Modalities

Affinity of bi- and tri-valent modalities of RBD-1-2G was determined against RBD-His (SinoBiological, 40592-V08H) using ForteBio amine reactive 2^{nd} generation (AR2G) biosensors. Biosensors were pre-hydrated in UltraPure DNase/RNase-free distilled water (Invitrogen, 10977015) for 10 minutes at 30° C. before use. A baseline in water for 60 seconds was followed by a 3 minute activation in a mixture of 20 mM 1-Ethyl-3-[3-dimethylaminopropyl] carbodiimide hydrochloride (EDC) and 10 mM N-hydroxysulfosuccinimide (s-NHS). Biosensor tips were then subjected to 2.5 μg/ml of RBD-His in kinetics buffer (PBS with 0.02% Tween20 and 0.1% BSA) for 150 seconds or until an average binding of ˜1.5 nm was reached. Sensors were quenched using 1 M ethanolamine (pH 8.5) for 150 seconds. Another baseline of 180 seconds in kinetics buffer was performed, before continuing to an association phase of 150 seconds. Analytes RBD-1-2G, RBD-1-2G-Fc and RBD-1-2G-Tri, were prepared at 100 nM in kinetics buffer, then diluted 1:2 until 6.125 nM concentration was reached. Disassociation was measured in kinetics buffer for 300 seconds. The 0 nM control wells were subtracted during data analysis. Data processing (Data Analysis HT 11.1) included alignment of curves to baseline 2 (last 5 seconds), inter-step correction aligned to dissociation, and Stavitzky-Golay Filtering. Data were analyzed using a 1:1 binding model, with global curve fitting used to calculate apparent affinities.

### SARS-COV-2 Mutant S1 Binding

Determination of RBD-1-2G-Fc ability to bind S1 mutant variants was determined by loading of mutant protein onto an anti-human IgG Capture (AHC) biosensor, then exposing them to various S1 modalities at 200 nM. RBD-1-2G-Fc was prepared at 10 μg/ml in kinetics buffer (PBS pH 7.4+0.02% tween and 0.1% protease-free BSA). Wildtype S1 (Sino Biological, 4059I-V08H) and B.1.1.7 variant (Sino Biological, 4059I-V08H12) were prepared at 200 nM in in kinetic buffer (PBS pH 7.4+0.02% tween and 0.1% protease-free BSA) or 10 mM acetate buffers with 150 mM NaCl (pH 4-pH 6.0). Biosensors were hydrated for 10 minutes in water, and the plate was preincubated for 10 minutes at 30° C. before the experiment started. Experimental parameters were baseline 1 minute, conditioning 20 seconds in 10 mM glycine (pH 1.5), neutralization 20 seconds (condition and neutralization performed 3 times each), loading 2 minutes, baseline 1 minute, association 1.5 minutes, dissociation 3 minutes. For data analysis, the 0 nM analyte with S1 protein loaded was subtracted from corresponding loaded sensors. Alignment of curves to the last 5 seconds of baseline 2, inter-step correction aligned to dissociation and Stavitzky-Golay filtering were used. Data were presented as the maximum response achieved during the association phase for the various pH conditions tested.

### AlphaLISA Assay

The ability of nanobodies to disrupt recombinant spike protein RBD binding to ACE2 was assessed using an AlphaLISA assay (Hanson et al., Acs Pharmacol Transl 3, 1352-1360, 2020). In dose response (1 μM-6 μM) nanobodies were preincubated with 4 nM SARS-COV-2 spike protein receptor binding domain fused to an Fc tag (RBD-Fc, SARS-COV-2 spike protein residues 319-541) (Sino Biological, Wayne, PA) in PBS supplemented with 0.05 mg/mL BSA at 25° C. for 30 minutes. After incubation, ACE2 with a C-terminal His and AviTag (ACE2-Avi, human ACE2 residues 18-740) (ACROBiosystems, Newark, DE) was added to a final concentration of 4 nM and the resulting mixture was incubated at 25° C. for 30 minutes. To produce the AlphaLISA signal a streptavidin donor bead, which recognizes ACE2-Avi, and Protein-A acceptor beads, which recognizes RBD-Fc, were added to a final concentration of 10 μg/ml each and the resulting mixture was incubated at 25° C. for 40 minutes in the dark. AlphaLISA luminescent signal was measured using a PheraSTAR (BMG Labtech, Cary, NC) plate reader with a 384-well format focal lens equipped with an AlphaLISA optical module (BMG Labtech, Cary, NC). All experiments were performed in triplicate.

### QD₆₀₈-RBD Neutralization Assay

QD₆₀₈-RBD was synthesized as previously described (Gorshkov et al., Acs Nano 14, 12234-12247, 2020). ACE2-GFP cells (25,000) were seeded into PDL-coated 96-well plates and incubated overnight. Nanobodies or nanobody-Fc constructs were pre-incubated with 10 nM QD₆₀₈-RBD for 30 minutes. Cells were washed 1× with Optimem I Reduced Serum Media before 3 hour treatment with 10 nM QD₆₀₈-RBD pre-incubated with nanobodies. Cells were imaged on the Perkin Elmer Opera using a 40× water immersion objective. 8-10 fields per well were captured from duplicate wells in a single plate. Approximately 1600-2500 cells were imaged per condition.

### Pseudotyped Particle (PP) Neutralization Assay

SARS-COV-2 pseudotyped particles (PPs) were purchased from Codex Biosolutions (Gaithersburg, MD), and were produced using a murine leukemia virus (MLV) pseudotyping system (Chen et al., Acs Pharmacol Transl 3, 1165-1175, 2020). All SARS-COV2-S constructs were C-terminally truncated by 19 amino acids to reduce ER retention for pseudotyping. The WTS sequence was the Wuhan-Hu-1 sequence (BEI #NR-52420). The variant B1.1.7 (Alpha) contains mutations del69-70, del144, N501Y, A570D, D614G, P681H, T716I, S982A, and D1118H.

The PP neutralization assays were performed as previously described (24). For WT PP assay, HEK293-ACE2 cells were seeded in white, solid bottom 1536-well microplates (Greiner BioOne) at 2000 cells/well in 2 μL/well media (DMEM, 10% FBS, 1×L-glutamine, 1×Pen/Strep, 1 μg/ml puromycin) and incubated at 37° C. with 5% CO₂ overnight (˜16 hours). Test articles were titrated 1:2 in PBS and acoustically dispensed to assay plates at 200 nL/well. Cells were incubated with test articles for 3 hours at 37° C. with 5% CO₂, before 2 μL/well of SARS-COV-2-S PP was added. The plates were then spinoculated by centrifugation at 1500 rpm (453×g) for 45 minutes, then incubated at 37° C. for 48 hours at 37° C. with 5% CO₂ to allow cell entry of PP and for the expression of luciferase reporter. After the incubation, the supernatant was removed with gentle centrifugation using a Blue Washer (BlueCat Bio). Then 4 μL/well of Bright-Glo Luciferase detection reagent (Promega) was added to assay plates and incubated for 5 minutes at room temperature. The luminescence signal was measured using a PHERAStar plate reader (BMG Labtech). For SARS-COV-2 variants PP neutralization assays, HEK293-ACE2 cells were seeded in white, solid bottom 384-well microplates (Greiner BioOne) at 6000 cells/well in 15 μL/well media. The cells were incubated at 37° C. with 5% CO₂ overnight (˜16 hours). Nanobodies were titrated 1:2 in PBS and added to cells at 1 μl/well. Cells were incubated with nanobodies for 1 hour at 37° C. 5% CO₂, before 15 L/well of SARS-COV-2 PP was added. The plates were then spinoculated by centrifugation at 1500 rpm (453×g) for 45 minutes, and incubated at 37° C. for 48 hours at 37° C. 5% CO₂ to allow cell entry of PP and expression of luciferase reporter. After the incubation, the supernatant was removed with gentle centrifugation using a Blue Washer (BlueCat Bio). Then 20 μL/well of Bright-Glo Luciferase detection reagent (Promega) was added to assay plates and incubated for 5 minutes at room temperature. The luminescence signal was measured using a PHERAStar plate reader (BMG Labtech). Data was normalized with wells containing SARS-COV-2 PP as 100%, and wells containing bald PP as 0%. An ATP content cytotoxicity assay was performed by omitting the PP and adding media instead. Data was normalized with wells containing cells as 100%, and wells containing media only as 0%.

### SARS-COV-2 Cytopathic Effect (CPE) Assay

The SARS-COV-2 cytopathic effect (CPE) assay was conducted in the BSL-3 facilities at Southern Research (Birmingham, AL), as previously described (Chen et al., Front Pharmacol 11, 592737, 2021). Briefly, nanobodies were titrated in PBS and added to 384-well assay plates at 3 μL/well to make assay-ready plates (ARPs), which were then frozen and shipped to the testing facility. Cell culture media (MEM, 1% Pen/Strep/GlutaMax, 1% HEPES, 2% HI FBS) was dispensed at 5 μL/well into ARPs and incubated at room temperature to allow for compound dissolution. Vero E6 African green monkey kidney epithelial cells (selected for high ACE2 expression) were inoculated with SARS-COV-2 (USA_WA1/2020) at a multiplicity of infection (MOI) of 0.002 in media, and quickly dispensed into assay plates as 25 μL/well. The final cell density was 4000 cells/well. Assay plates were incubated for 72 hours at 37° C., 5% CO₂, and 90% humidity. CellTiter-Glo (30 μL/well, Promega #G7573) was dispensed into the assay plates. Plates were incubated for 10 minutes at room temperature. Luminescence signal was measured on Perkin Elmer Envision or BMG CLARIOstar plate readers. Data was normalized with buffer-only wells as 0% CPE rescue, and no-virus control wells as 100% CPE rescue. An ATP content cytotoxicity counter-assay was conducted using the same protocol as the CPE assay, without the addition of SARS-COV-2 virus. Data was normalized with buffer-only wells as 100% viability, and cells treated with hyamine (benzethonium chloride) control compound as 0% viability.

### Membrane Protein Array Assay

Membrane Proteome Array (MPA) screening was conducted at Integral Molecular (Philadelphia, PA). The MPA is a protein library composed of 6,000 human membrane protein clones, each overexpressed in live cells from expression plasmids. Each clone was individually transfected in separate wells of a 384-well plate followed by a 36-hour incubation (Tucker et al., Proc Natl Acad Sci USA 115, E4990-E4999, 2018). Cells expressing each individual MPA protein clone were arrayed in duplicate in a matrix format for high-throughput screening. Before screening on the MPA, the test antibody (RBD-1-2G) concentration for screening was determined on cells expressing positive (membrane-tethered Protein A) and negative (mock-transfected) binding controls, followed by detection by flow cytometry using a fluorescently-labeled secondary antibody. Each test antibody was added to the MPA at the predetermined concentration, and binding across the protein library was measured on an Intellicyt iQue using a fluorescently-labeled secondary antibody. Each array plate contains both positive (Fc-binding) and negative (empty vector) controls to ensure plate-to-plate reproducibility. Test antibody interactions with any off targets identified by MPA screening were confirmed in a second flow cytometry experiment using serial dilutions of the test antibody, and the target identity was re-verified by sequencing.

### SARS-COV-2 Prefusion S-Protein Ectodomain

The construct VRC 7471 (Dale and Betty Bumpers Vaccine Research Center, NIAID) encodes for nCOV S-2P-dFu-F-3C-H-2S,S ectodomain, with proline substitutions at residues 986 and 987, the furin site mutated to GSAS (SEQ ID NO: 10), a T4 fibritin trimerization motif, a PreScission protease cleavage site, and 8×His and Strep tags. It is based on the construct used for an early published structure (Wrapp et al., Science 367, 1260-1263, 2020). Expression was carried out in Expi293 cells (Thermo Fisher Scientific) following the manufacturer's protocol. Briefly, 1 L Expi293 cells were transiently transfected using ExpiFectamine and incubated at 37° C. for 18 hours. At this point enhancers were added and the temperature shifted to 32° C. for 96 hours. To harvest the secreted protein, cells were pelleted by centrifugation at 400 g for 15 minutes and the supernatant was filtered through a 0.45 μm filter. A total of 5 ml of Talon metal affinity resin (Takara Bio USA) was added to the supernatant and mixed overnight at 4° C. The supernatant/resin mixture was gravity loaded on a column and washed with 100 ml 50 mM Tris pH 8.0/150 mM NaCl followed by 100 ml of 50 mM Tris pH 8.0/500 mM NaCl. The spike trimer was batch eluted with 50 mM Tris pH 8.0/150 mM NaCl/200 mM Imidazole. Fractions containing spike trimer, as assessed by SDS-PAGE, were pooled and buffer exchanged/concentrated to ˜1 mg/ml using Amicon Ultra 4 centrifugal filters (Millipore Inc). Concentrated protein was divided into small aliquots, flash frozen in liquid nitrogen and stored at −80° C.

### Cryo-EM Structure Determination of the Nanobodies Bound to the Trimeric Spike Protein Specimen Preparation

To increase hydrophilicity, grids were pretreated in a Tergeo EM plasma cleaner (PIE Scientific) under an argon atmosphere for 1 minute, using the immersion mode at 25w. A 3 μL aliquot of 2.73 μM SARS COV-2 S in Tris pH 8.0 50 mM and 160 mM NaCl buffer, mixed with each nanobody (Nb) was applied onto a clean UltrAufoil R1.2/1.3 300-mesh grid. Excess solution was removed by blotting with Whatman No. #1 prior to plunging into liquid ethane (−182° C.) in a Leica EM GP 2 (Leica) vitrification robot (automated blot pressure, chamber at 25° C. and 95% RH). The RBD-1-1G (1.5 mg/ml), RBD-1-2G (1.3 mg/ml), RBD-2-1F (1.8 mg/ml), RBD-1-3H (1.2 mg/ml), RBD-2-1B (1.7 mg/ml) and RBD-2-3A (4.2 mg/ml) were prepared in PBS (pH 7.4).

### Data Collection

Data was collected on either a Titan Krios or in a Talos Arctica transmission electron microscope (TFS) operated at 300 and 200 KeV respectively. The former was equipped with a post column energy filter (Gatan) and operated with a 20 eV slit size. Multiframe images were collected on direct electron detectors (Gatan K2). Details for each data set are described in Table 1.

TABLE 1
Data collection parameters for each data set

| | | | | | Exposure | | |
|---|---|---|---|---|---|---|---|
| | Voltage | Mag | Pixel Size | Underfocus | Rate | Total | Movies |
| Data set | (KeV) | (kX) | (Å) | Range (Å) | (e/px/s) | Dose | Collected |
| RBD-1-2G | 300 | 130 | 0.532 (SR) | 1.2-2.4 | 8 | 60 | 4230 |
| RBD-1-1G | 200 | 36 | 1.187 (CM) | 1.2-2.2 | 8 | 60 | 2743 |
| RBD-1-3H | 200 | 36 | 1.187 (CM) | 1.5-2.8 | 8 | 60 | 4977 |
| RBD-2-1F | 200 | 36 | 1.187 (CM) | 1.2-2.8 | 8 | 60 | 3347 |
| RBD-2-1B | 200 | 36 | 1.187 (CM) | 1.4-2.4 | 8 | 60 | 1693 |
| RBD-2-3A | 200 | 36 | 1.187 (CM) | 1.5-2.2 | 8 | 60 | 1755 |

*SR—Super Resolution
**CM—Counting Mode

### Image Processing

All image processing was carried out in the context of RELION-3.1.0. Motion correction was performed using the internal implementation and standard parameters without binning. CTF was estimated using CTFFIND4 in a resolution range of 3.0-30 Å, defocus range of 5000-35000 Å and a defocus step size of 500 Å. Laplacian-of-Gaussian detection with minimum and maximum diameters of 150 and 180 Å was used to pick particles. These were extracted using a box size of 300 pixels and Fourier down-sampled to 100 pixels (final size 3.562 A/pixel). Contamination and outliers were initially removed running 2 or 3 rounds of 2D classification. “Clean” particles were used to generate an initial 3D map using the stochastic gradient descent algorithm. Further elimination of outliers was achieved using 3D classification. A consensus C3 symmetric map was refined from each clean dataset and used for full defocus refinement (anisotropy, defocus and higher order aberration). A variety of approaches to classification (with/without alignment, symmetry expansion, etc.) were taken after this step in order to improve the resolution of the asymmetric portions of the spike.

### Cryo-EM Model Building and Analysis

An atomic model for the C3 symmetric closed structure of the SARS-COV-2 spike protein (pdb: 6zp0) was used as the starting point for the fitting in the cryo-EM maps. To generate the “one-up” model, one of the RBDs in 6zp0 (residues 336-520) was manually aligned to the open state using pdb: 6vyb as reference. The atomic model of the spike protein was positioned as a rigid body into each map using Fit in map tool on Chimera. After this, the atomic models were flexibly fitted to the maps using default real space refinement strategies (local grid search, global minimization, morphing and atomic displacement parameters refinement) on Phenix 1.18.2. This refinement was performed without either secondary restrains nor refinement of NCS operator and while applying Ramachandran restrains. The maximum number of iterations was increased to 150. The Ramachandran plot and Fourier Shell Correlation plot between the atomic model simulated map and the cryo-EM map, were used to validate the fitting. In order to obtain the proper binding mode between each nanobody with the RBD, molecular docking calculations were combined with flexible fitting into the cryo-EM maps as follows. The 3D structural models of nanobody were generated using the I-TASSER program (Roy et al., Nat Protoc 5, 725-738, 2010). The top 10 template structures of antibodies used by the threading program share 60-70% of sequence identities with the nanobody. All generated models showed a good quality in general (C-score>0 and Z-score>1). The best structural model was further refined with energy minimization and molecular dynamic (MD) simulations using the Amber20 program (Case et al., University of California, San Francisco, 2020). After fitting the spike atomic model to each density map, the Nb bound conformation of the RBD was extracted from the atomic model. The binding mode between nanobodies RBD-1-3H, RBD-2-1F, RBD-1-1G and RBD-1-2G to the corresponding fitted RBD was then predicted using ZDOCK server. All ten binding modes for each Nb-RBD complex reported by ZDOCK were fitted as rigid bodies to the corresponding RBD density maps in UCSF Chimera. Models best correlating with the map were then refined using the Cryo_fit routine in Phenix (default parameters for version 1.18-3855).

### Molecular Dynamics

To better understand the interaction of RBD-1-2G with the RBM region, atomic model fitting was performed to identify the key binding residues. Since the resolution of the RBD region in the map was not high enough to directly derive an atomic model, fitting the atomic models of RBD and RBD-1-2G into the low-resolution map was the next best solution. However, the apparent symmetry of VHHs at low resolution leads to ambiguous assignments of their orientation in the context of the complex. A model of the spike (6zp0) was fitted into the map and the portion corresponding to the RBD coordinates was extracted (residues 328 to 531). Molecular docking calculations, where the RBD coordinates were kept rigid while the nanobodies' coordinates were allowed to fluctuate were performed using the ZDOCK platform. Out of ten conformations requested, a pair that showed CDRs facing the RBM was positioned in the map relative to the RBD in the “half down” state of the spike model. The simulated density of the two complexes were compared with the map and the result exhibiting the highest correlation coefficient (CC) was selected for further refinement. This atomic model fitting strategy combined with molecular docking using the Cryo-EM map as a filter to choose the best predicted binding modes was tested against the nanobodies RBD-1-2G, RBD-2-1F, RBD-1-1G, and RBD-1-3H and showed successful predictions in all the cases.

Molecular dynamics simulations were performed on RBD-1-2G and WT RBD as well as RBD-1-2G and the alpha variant of RBD complexes to further characterize the energetics and the involvement of various residues located in the interacting interface. Starting protein structures were obtained from the cryo-EM density matched molecular docking. All MD simulations were performed using Amber. 18 (Wang et al., Nature 593, 130-135, 2021) with the ff14SB force field representing protein atoms. Initial minimization, equilibration, and all production runs were carried out with the GPU enhanced PMEMD module of Amber.18. Initial coordinates and topology files were generated using the Leap module, with the selection of rectangular TIP3P water box solvating the protein complexes. The box boundaries were extended at least 20 Å from the solute. Both systems contained 57 Na⁺ ions and 61 Cl⁻ ions that provided the charge neutralization and the 100 mM salt concentration. The WT system was comprised of 97,998 atoms and the mutant system had 98,006. After the initial equilibration of water and ions with each protein system was subjected to positional restraints with the 100 kcal/mol/Å² force constant, a minimization was followed with the same force constants on the protein atoms. While maintaining the force constants at 10 kcal/mol/Å², each system was subjected to a 2 ns low temperature (T=100 K) constant pressure simulation to get the system density adjusted to a realistic value. After a step-wise slow heat-up to 300 K within 1 ns, each system was further equilibrated for up to 10 ns with the position constraints on the protein atoms. Within the next 10 ns the positional constants were removed step-wise, and each system was subjected to a further 10 ns of equilibration. Three independent equilibrations were performed on each system (with the starting conformations for the second and third runs selected from the 10th and 20th ns configurations of the first MD run). The constant pressure production runs were performed for 500 ns with the 2 fs time step for all systems. The particle mesh Ewald method was used in the treatment of long-range electrostatics with the short-range cut-off of 9 Å. The MMGBSA module of Amber.18 was implemented in free energy estimations with the selection of 0.15 M salt concentration and the default parameters (IGB=5) within the Amber module. 500 configurations selected at each nanosecond of each trajectory were used in this calculation. All other analysis was done using the CPPTRAJ module of Amber.18.

### Example 2: Identification of SARS-COV-2 Neutralizing VHHs from Synthetic, Humanized Nanobody Libraries

To develop a platform for rapid in vitro nanobody discovery, a number of synthetic phage-displayed nanobody libraries were designed, starting from a backbone derived from the first approved humanized nanobody drug caplacizumab (U.S. Pat. No. 10,919,980) (FIG. 1A). It was hypothesized that by basing the library on the caplacizumab framework that has been optimized for in-human use, that potent binders identified through phage panning could be rapidly progressed for investigational new drug (IND) studies and trials. The caplacizumab framework region was combined with synthetized complementarity-determining loops (CDRs) to diversify the highly variable antigen-binding interface of the nanobody (McMahon et al., Nat Struct Mol Biol 25, 289-296, 2018). This resulted in library diversity of >10¹⁰ transformants that could theoretically be rapidly translated to human trials. Phage panning was performed for one round against S1 protein of SARS-COV-2, with two additional panning rounds against RBD of SARS-COV-2 (FIG. 1B). A total of 13 nanobodies were identified during the panning process, with 10 of these showing sequence enrichment.

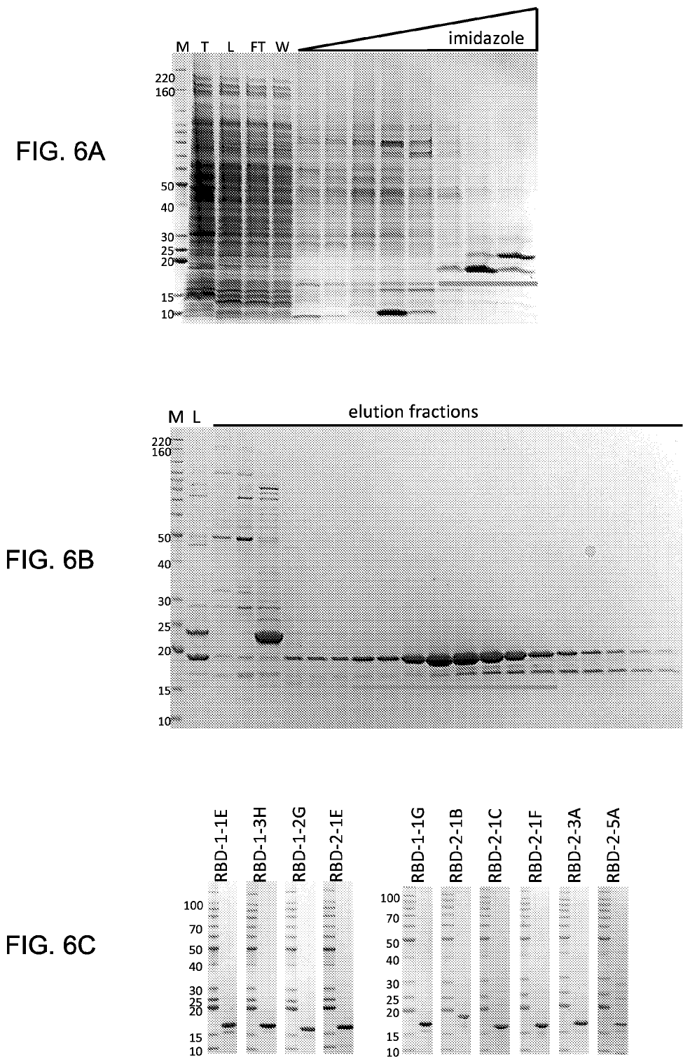

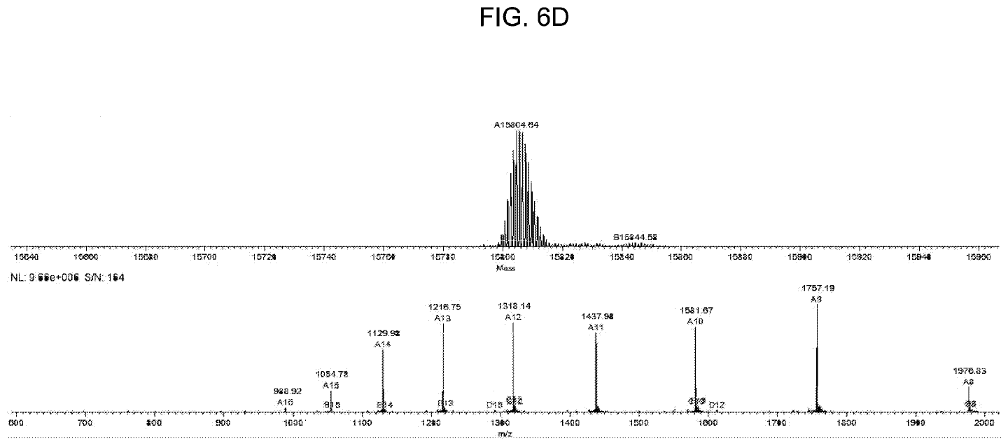

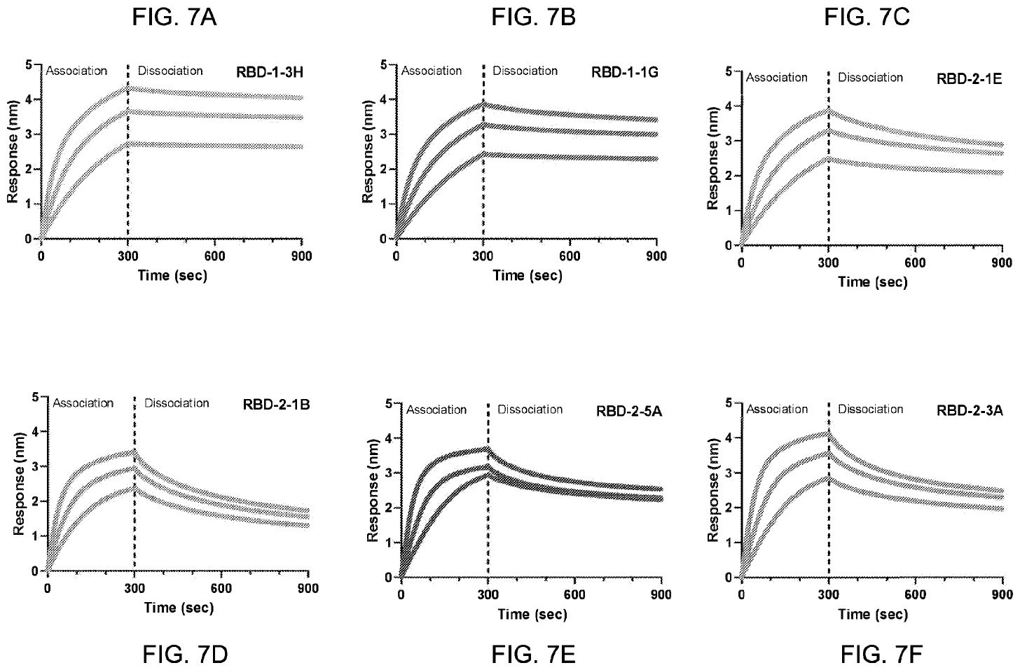

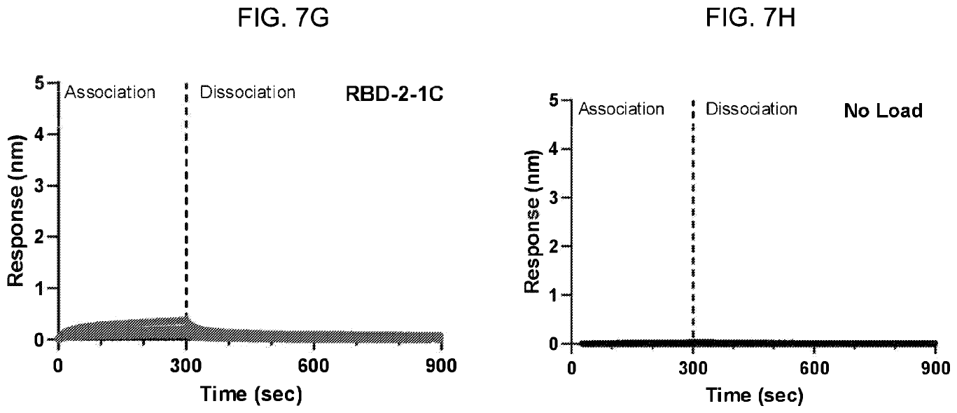

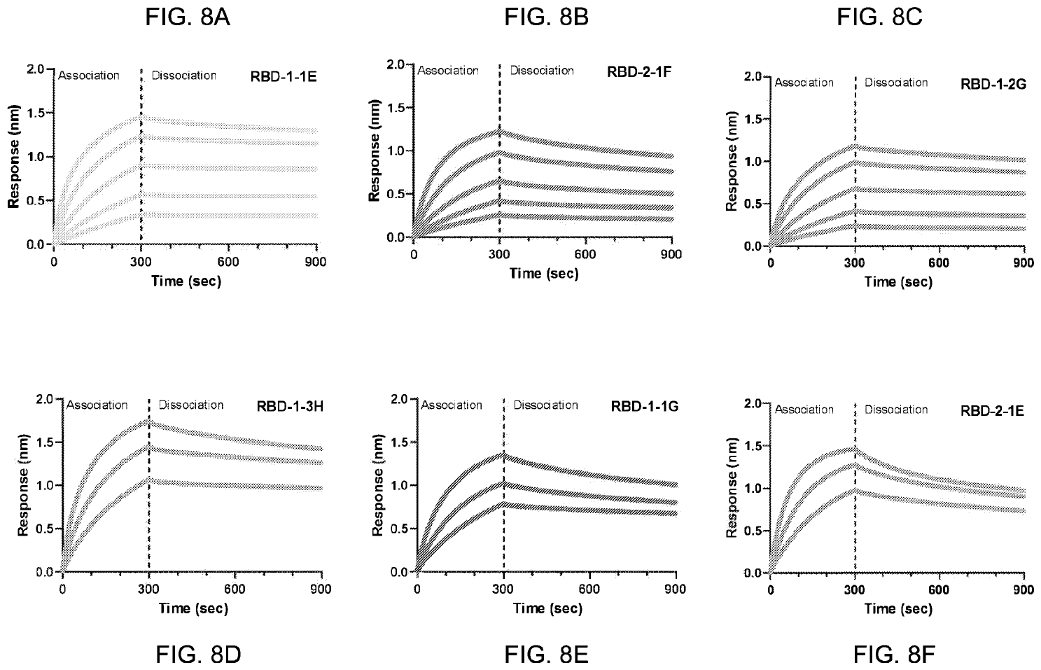

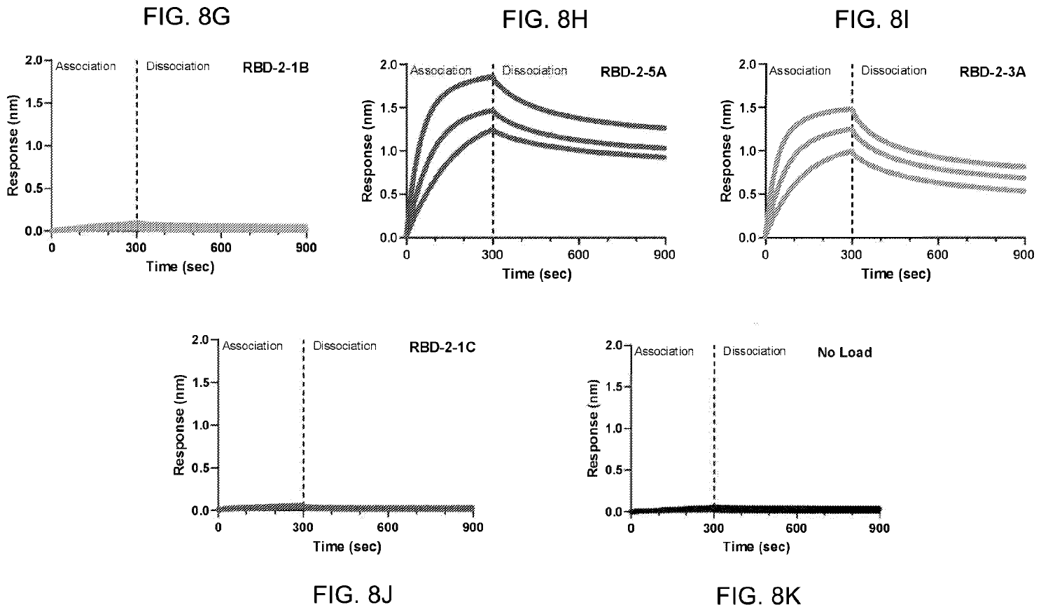

Nanobodies were produced in Vibrio nutriegens and purified using Ni-NTA affinity chromatography followed by size exclusion chromatography (SEC). Nanobody size and MW were confirmed by SDS-PAGE and high-resolution MS (FIGS. 6A-6D). Octet analysis was used to visualize real time association and dissociation of RBD-mFc and S1-hFc to the various nanobodies. Global fit modeling revealed binding affinities of 0.9 nM (RBD-1-1E), 4.9 nM (RBD-2-1F) and 9.4 nM (RBD-1-2G) towards RBD-mFc (FIGS. 1C-1D, FIGS. 7A-7H). When S1-hFc was used as the analyte, RBD-1-2G showed an affinity of 6.9 nM, a 26.6% improvement (6.9 nM vs 9.4 nM) over the RBD-mFc analyte. Reduced binding affinity was observed for both RBD-2-1F (24.6 nM vs 4.9 nM) and RBD-1-1E (3.8 nM to 0.9 nM) when switching to the S1 format (FIG. 1D, FIGS. 8A-8K). The observed differences on affinity are probably due to some epitopes on RBD being less accessible in the context of dimerized S1. To evaluate whether these nanobodies inhibit RBD-ACE2 complex formation, an AlphaLISA assay was developed (Hanson et al., Acs Pharmacol Transl 3, 1352-1360, 2020). ACE2-Avi and RBD-Fc association was determined in the presence of increasing concentration of nanobodies to evaluate their receptor-blocking capabilities (FIG. 1E). Despite RBD-1-2G having the second highest affinity for S1-hFc, it yielded a lower IC₅₀ than RBD-1-1E (28.3 nM vs. 126 nM) (FIG. 1E). These results support RBD-1-2G as a potential candidate for therapeutic development.

### Example 3: Multimerization of RBD-1-2G to Improve Neutralization of SARS-COV-2 Virus

To further evaluate RBD-1-2G and for improved SARS-COV-2 neutralization performance, bivalent and trivalent modalities were constructed. The bivalent format, RBD-1-2G-Fc, had an improved binding affinity of 1.9 nM compared to the monovalent format with 14.3 nM (FIG. 2A, FIG. 2B). The trivalent format (RBD-1-2G-Tri) was constructed through the linking of three monovalent formats with (GGGGS) 3 (SEQ ID NO: 9) flexible linkers. This further improved the apparent affinity to 0.1 nM for RBD binding (FIG. 2C).

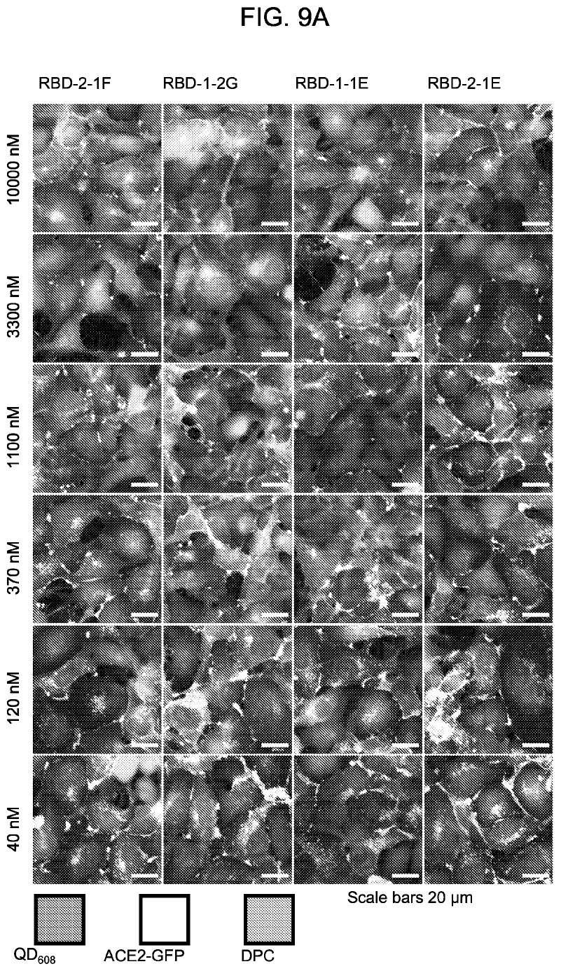

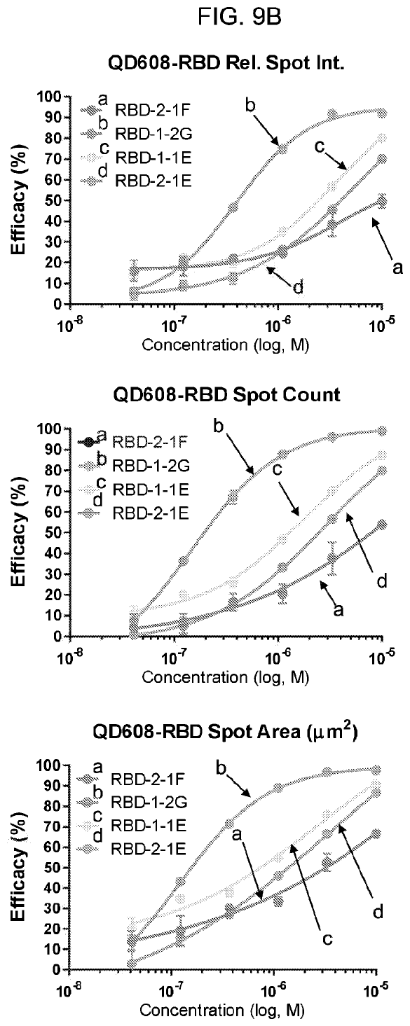

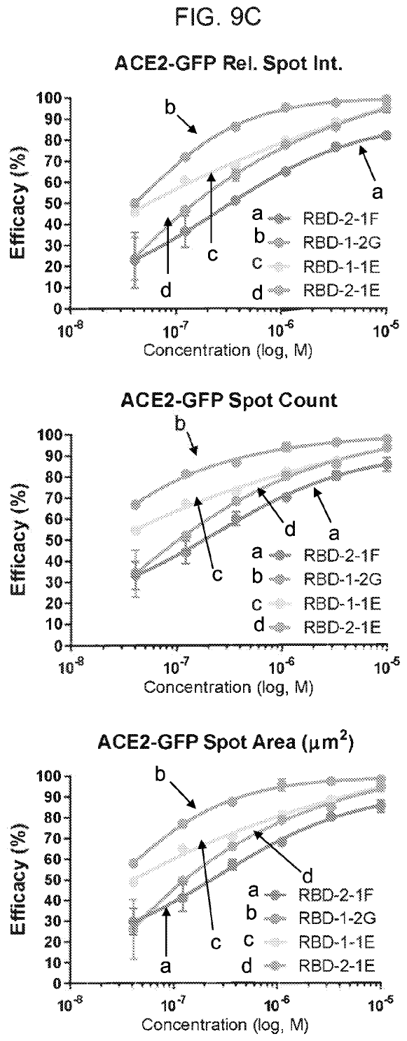

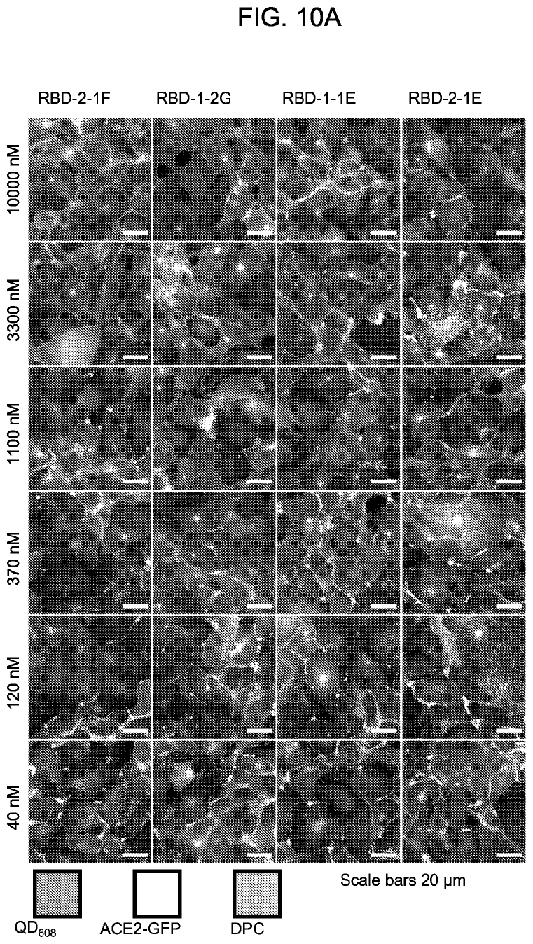

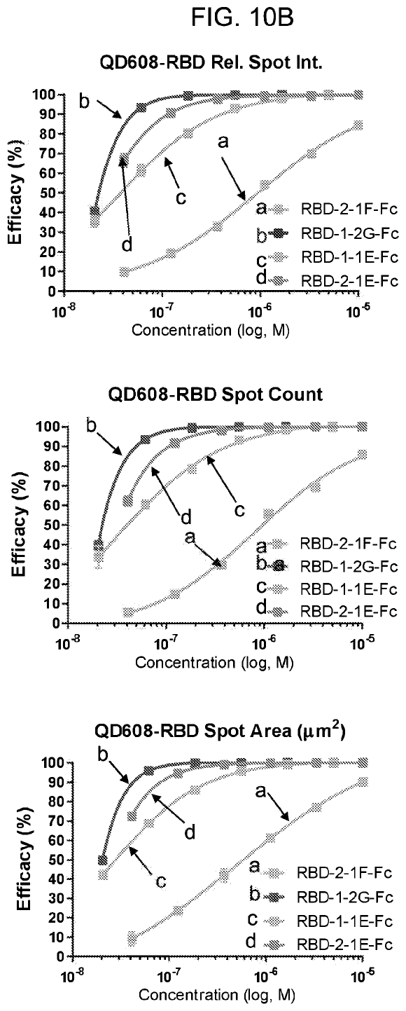

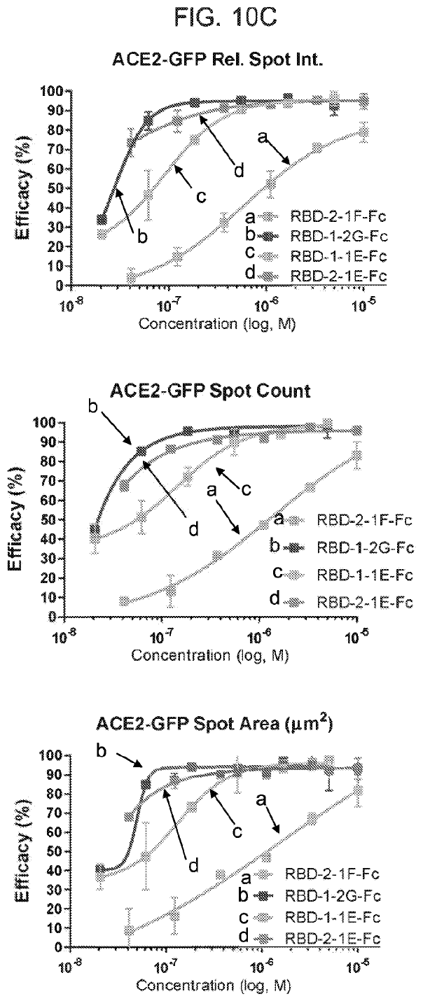

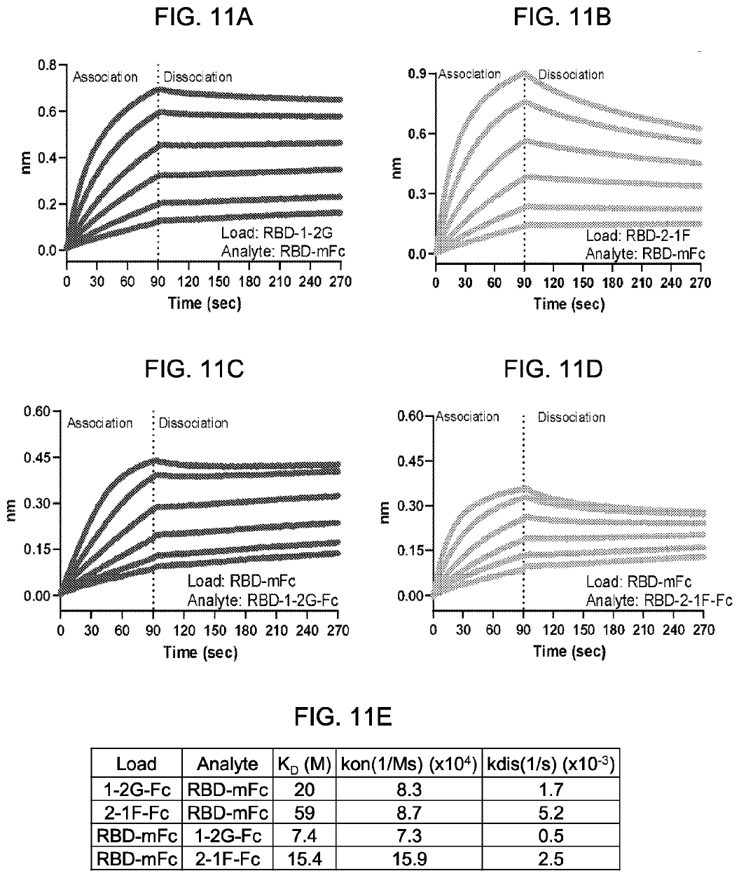

To test if this improved affinity would translate to improved therapeutic potential, an endocytosis assay was performed using SARS-COV-2 RBD quantum dots (QD₆₀₈-RBD) (FIGS. 9A-9C, FIGS. 10A-10C) (Gorshkov et al., Acs Nano 14, 12234-12247, 2020). RBD-1-2G showed the highest potency with an IC₅₀ of 390 nM (FIG. 2D). Conversion of RBD-1-2G to the Fc format reduced the IC₅₀ to 14 nM (FIG. 2E). A similar trend was observed for RBD-1-1E-Fc (3728 nM to 32 nM), but not for RBD-2-1F-Fc (947 nM to 3883 nM) (FIG. 2D, FIG. 2E). Despite RBD-2-1F having a better affinity than RBD-1-2G (4.9 nM vs 9.4 nM, FIG. 1D), RBD-1-2G-Fc showed higher affinity than RBD-2-1F-Fc (FIGS. 11A-11E). The binding of RBD-2-1F to RBD may be inhibited by the presence of a Fc domain.

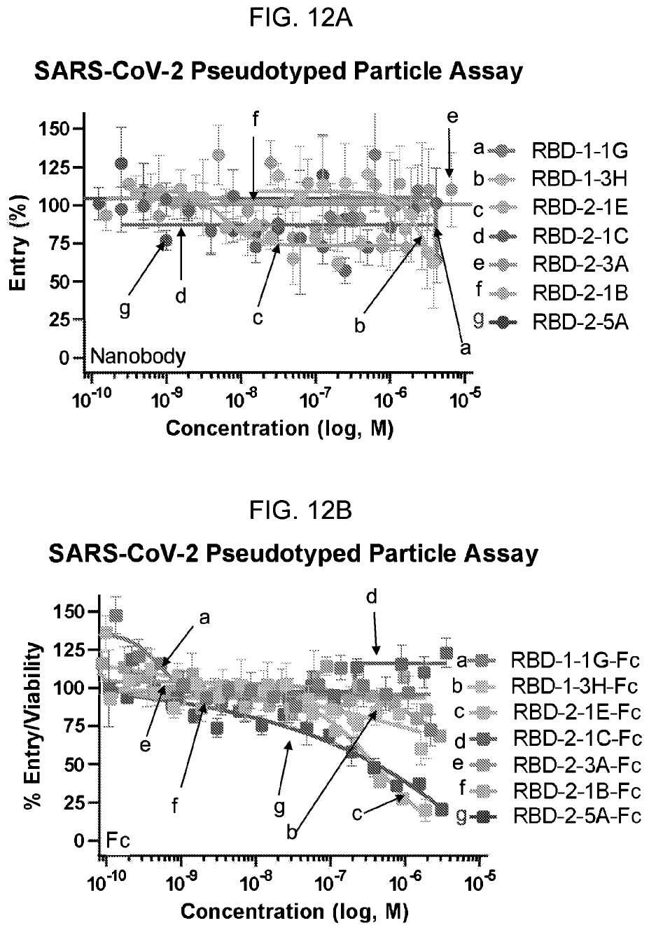

In vitro neutralization assays using SARS-COV-2 S-protein pseudotyped murine leukemia virus (MLV) vector particles were employed to further characterize the antiviral activity (Chen et al., Acs Pharmacol Transl 3, 1165-1175, 2020). RBD-1-2G was found to neutralize SARS-COV-2 pseudotyped viruses with an IC₅₀ of 490 nM (FIG. 2F). Significant improvements were seen with the Fc and trimer modalities showing inhibition at 88 nM (RBD-1-2G-Fc) (FIG. 2G). Consistent with the QD internalization assay, RBD-2-1F-Fc showed improvement over RBD-2-1F but failed to neutralize better than the RBD-1-2G modalities (FIG. 2F). The other nanobodies and Fc modalities tested showed little activity (FIGS. 12A-12B). A SARS-COV-2 live virus screen was performed to assess the neutralizing effect of nanobodies. RBD-1-2G-Tri produced an IC₅₀ of 182 nM, followed closely by RBD-1-2G-Fc with an IC₅₀ of 255 nM (FIG. 2H). The trimer and Fc modalities of RBD-1-2G were found to be more potent than RBD-1-2G (IC₅₀=1211 nM) and the RBD-2-1F-Fc (IC₅₀=3574 nM). These data support RBD-1-2G and its multimeric modalities for further development as SARS-COV-2 therapeutics.

### Example 4: Cryo-EM Revealed Two Distinct Modes of Binding for Neutralizing Nanobodies

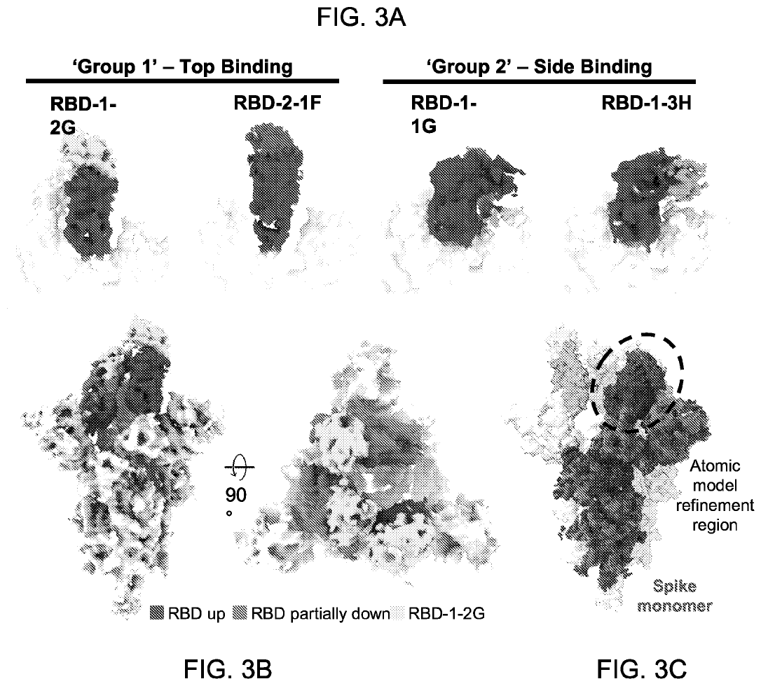

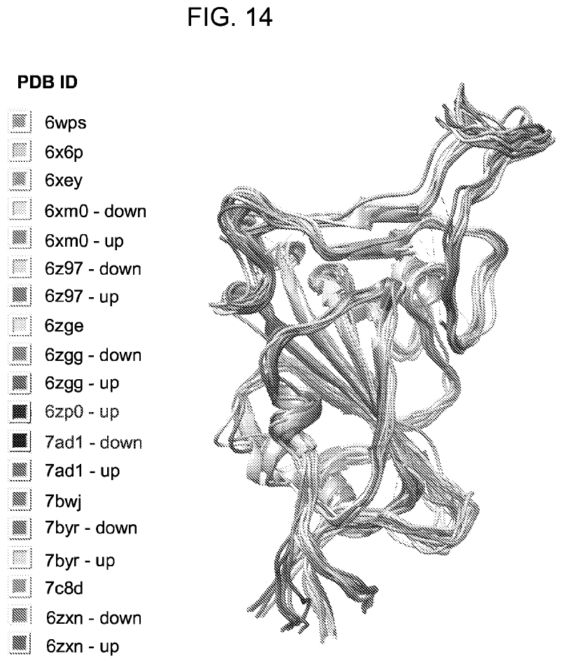

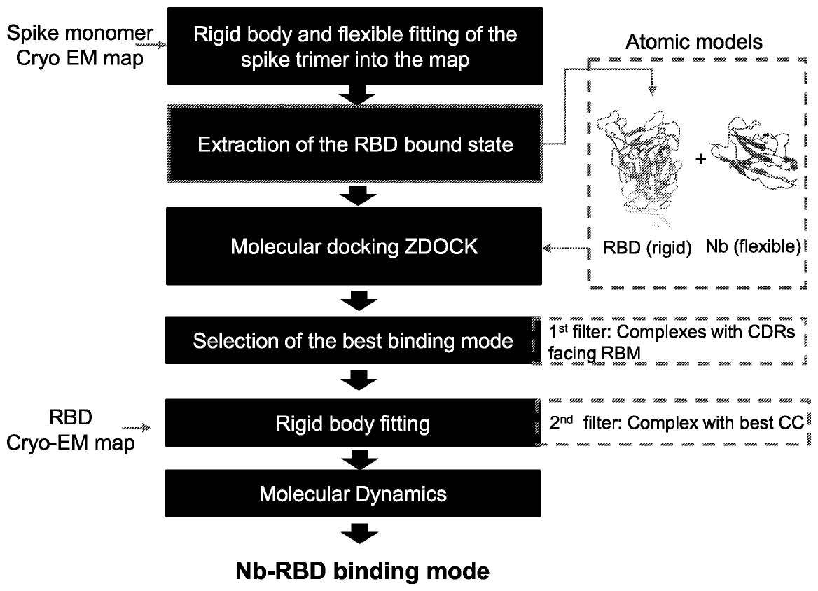

To uncover the region bound by the selected nanobodies, single particle cryo-EM was used to obtain electron scattering density maps of a soluble form of S-protein ectodomain in complex with 4 nanobodies (FIGS. 13A, 13B, 14 and 15). Maps of the complexes with RBD-1-2G, RBD-2-1F, RBD-1-1G and RBD-1-3H featured extra density in the RBD region (FIG. 3A). Two additional nanobodies (RBD-2-3A and RBD-2-1B) were tested, but the observed electron densities were found to be similar to unliganded spike. Low affinity was also observed when these nanobodies bound S1-hFc by Octet (FIG. 1D), suggesting their epitopes are not accessible in the context of the trimeric spike.

The accessible epitopes can be classified into two groups. ‘Group 1’ includes the binding area for RBD-1-2G and RBD-2-1F located at the distal end of the ‘up’ RBD in an area that overlaps with the RBM. Nanobodies RBD-1-1G and RBD-1-3H bind to epitopes in ‘group 2’, exposed on the external face of the erect RBD in an area not overlapping the RBM (FIG. 3A). The atomic model fitting of the spike (PDBID: 6zp0) suggests that the ‘group 1’ binders overlap with the ACE2 binding site, while ‘group 2’ binders do not prevent ACE2 binding (FIGS. 13A-13B). In the down conformation, epitopes of ‘group 2’ on the RBD are sterically occluded by the NTD of the neighboring monomer, thus nanobodies of this group were only detected when bound to the RBD in the ‘up’ conformation. In contrast, densities corresponding to RBD-1-2G can be detected in the ‘up’ and ‘half down’ conformations of the RBD. This unique intermediate between the ‘up’ and ‘down’ states allows for the accommodation of three molecules of RBD-1-2G without steric hindrance (FIG. 3B), likely explaining the high activity for RBD-1-2G. In order to improve resolution of the binding area, independent refinement of “RBD down” spike monomers (shown in maroon in FIG. 3C) was carried out using symmetry expansion and signal subtraction with the spike monomer.

### Example 5: RBD-1-2G Binds to WT and B.1.1.7 Variant (Alpha) at Various Physiological pHs

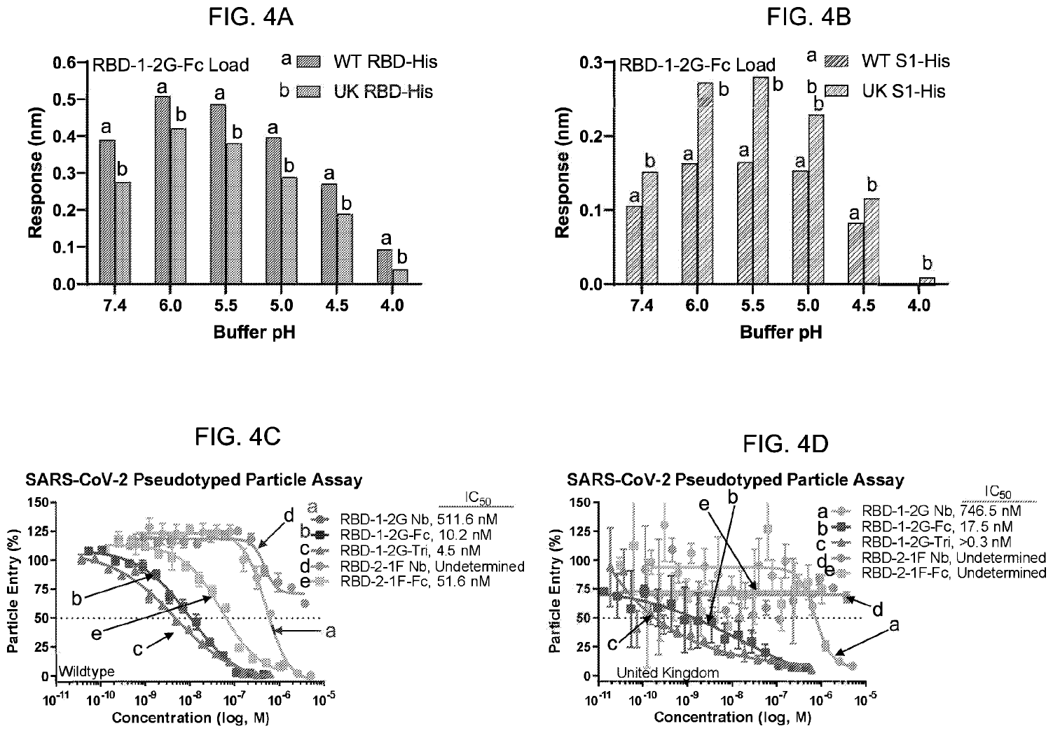

Different strains of SARS-COV-2 with the N501Y mutation have been identified, which have been associated with increased transmissibility and a reduction in neutralizing antibody activity (Dejnirattisai et al., Cell 184, 2939-2954, 2021; Wang et al., Nature 593, 130-135, 2021). Octet biosensor-immobilized RBD-1-2G-Fc was exposed to both wildtype and B.1.1.7 mutant (N501Y) S1-His. The maximum binding response to WT or B.1.1.7 RBD-His during the association phase was graphed over a range of pH conditions (7.4-4.0) (FIG. 4A). Similar binding profiles were observed for WT and B.1.1.7, but a stronger signal was observed with the mutant. Strongest binding was observed between pH 5.0 and 6.0. Binding was detectable down to a pH of 4.5. A similar pattern was observed when RBD-His was used instead of S1-His (FIG. 4B). The best binding was observed between pH 6.0 with a gradual reduction as pH was lowered to 4.0. These data suggest that RBD-1-2G stays bound to SARS-COV-2 WT and B.1.1.7 variant (Alpha) after internalization while in the endosome (pH=6.5-4.5).

The effects of B.1.1.7 spike mutants on viral particle neutralization were assessed. RBD-1-2G-Tri was found to inhibit the B.1.1.7 pseudotyped particles better than the wildtype particles (0.3 nM vs 4.5 nM) (FIG. 4C, FIG. 4D). RBD-1-2G-Fc (17.5 nM vs 10.2 nM) and RBD-1-2G (746.5 nM vs 511.6 nM) showed similar IC₅₀ against the B.1.1.7 variant (FIG. 4C, FIG. 4D). Additionally, RBD-2-1F-Fc was able to inhibit the WT pseudotyped particles but failed to inhibit the B.1.1.7 (Alpha) mutant version (FIG. 4C, FIG. 4D).

### Example 6: RBD-1-2G Displays Low Off-Target Binding

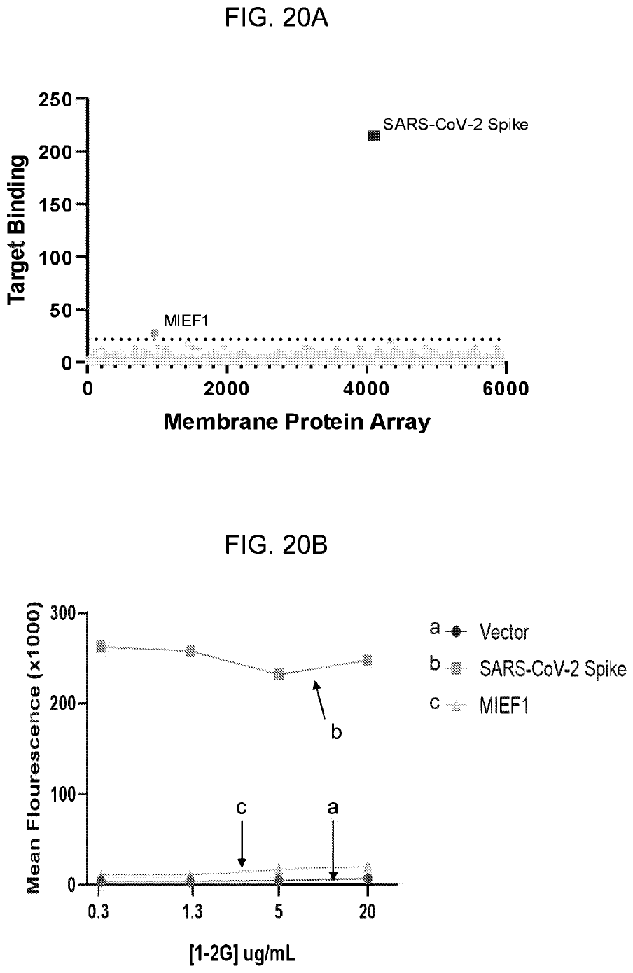

A human membrane proteome array (MPA, approximately 6,000 human membrane proteins +SARS-COV-2 spike protein) was used to screen and evaluate potential poly-reactivity and non-specificity of RBD-1-2G. The MPA screen confirmed high-specificity binding of RBD-1-2G to SARS-COV-2 spike with minimal binding to extraneous human membrane proteins (FIG. 20A). Binding to mitochondrial elongation factor 1 (MIEF1), an intracellularly expressed protein, was detected in an initial screen. Validation of this target revealed that RBD-1-2G bound MIEF1-transfected cells in a similar fashion to vector-transfected cells (FIG. 20B). Additionally, since MIEF1 is a mitochondrial protein, it will be unlikely to interfere with a blood infusion or nebulized administration of RBD-1-2G. These assays indicate minimal potential off-target effects of RBD-1-2G.

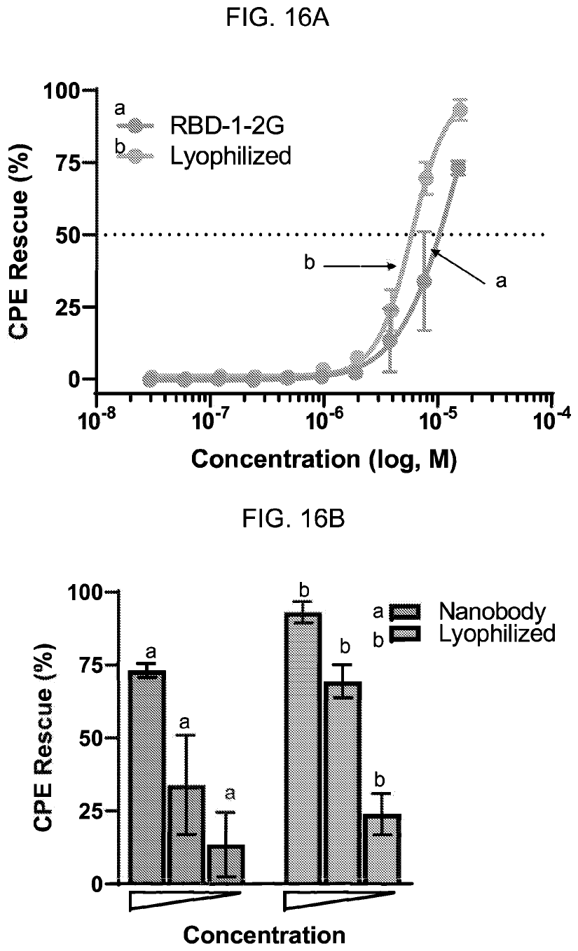

Additionally, RBD-1-2G nanobody was lyophilized and the reconstituted nanobody was tested in a similar live virus study. The lyophilization process had little effect on overall activity in the live virus screen (FIGS. 16A-16B), with IC₅₀ measuring 5.9 μM for the lyophilized and 14.8 μM in untreated samples (FIGS. 16A-16B). The ability of RBD-1-2G to retain activity after lyophilization is a positive feature that can be useful in the manufacturing and formation of nanobody-based therapeutics.

### Example 7: Atomic Modeling of RBD-1-2G Nanobody RBM Region Interaction

To elucidate the residues that participate in the binding of RBD-1-2G with the RBD regions of WT and B.1.1.7 (Alpha) variant, molecular docking of the RBD-Nb complex was employed before fitting it in the context of the spike. The best binding mode was selected by superimposing the docking results with the cryo-EM maps. Next, the solvated RBD-1-2G-WT RBD and RBD-1-2G-B.1.1.7 RBD complexes were subjected to over 1 us molecular dynamic (MD) simulations in solution.

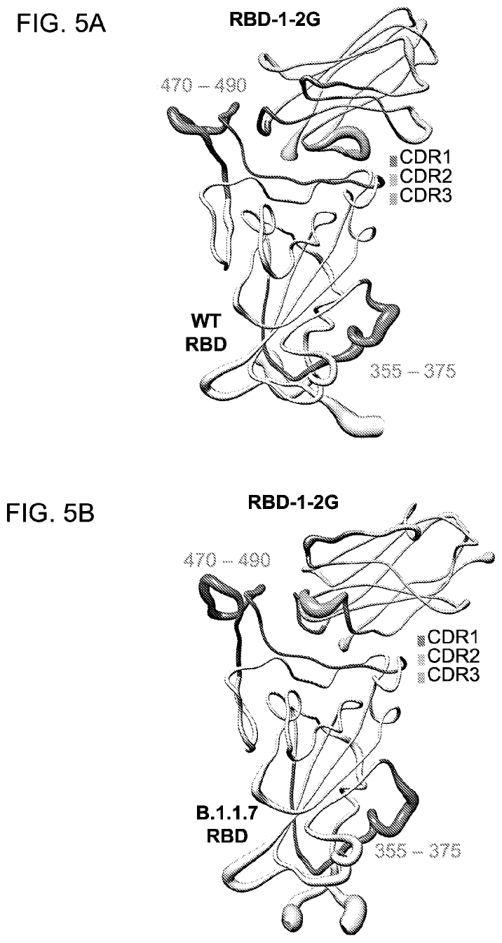

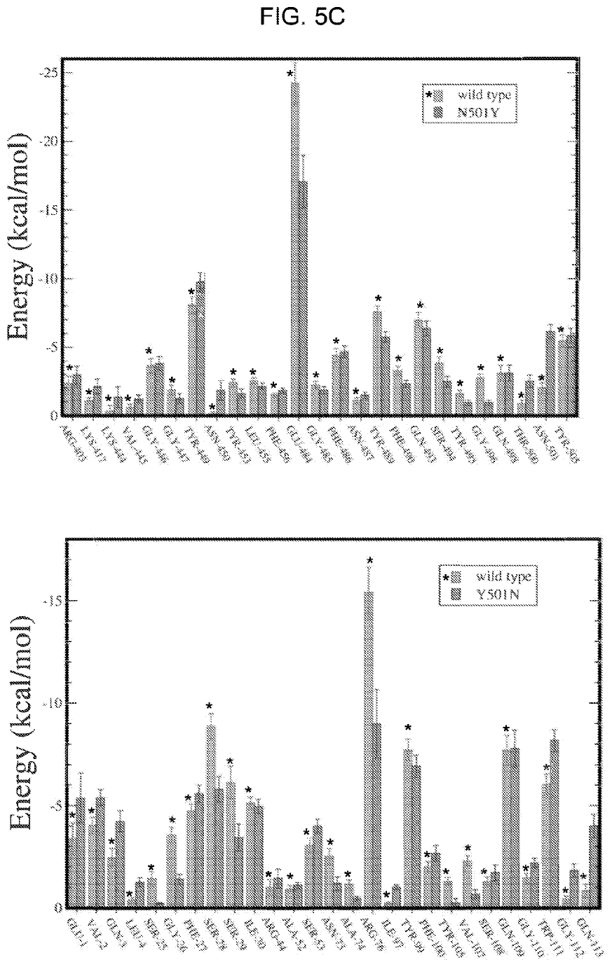

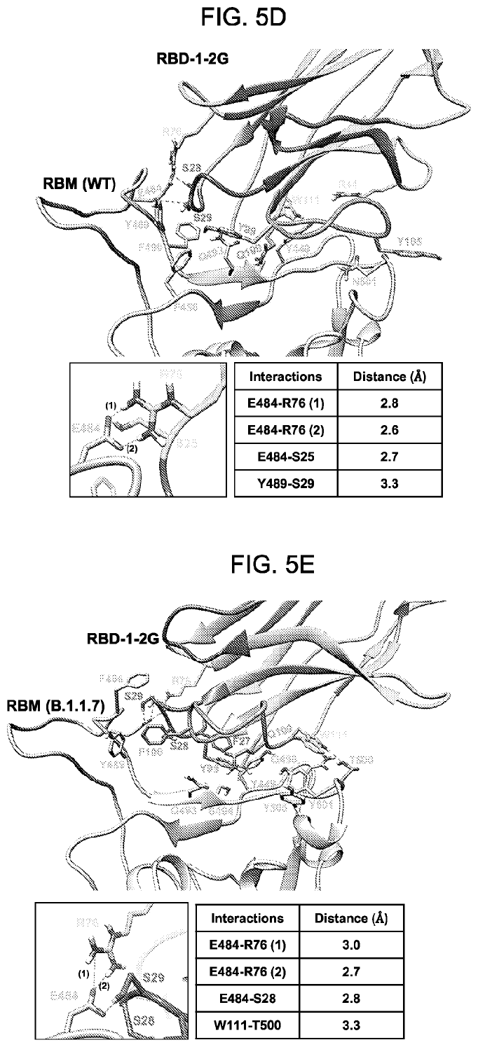

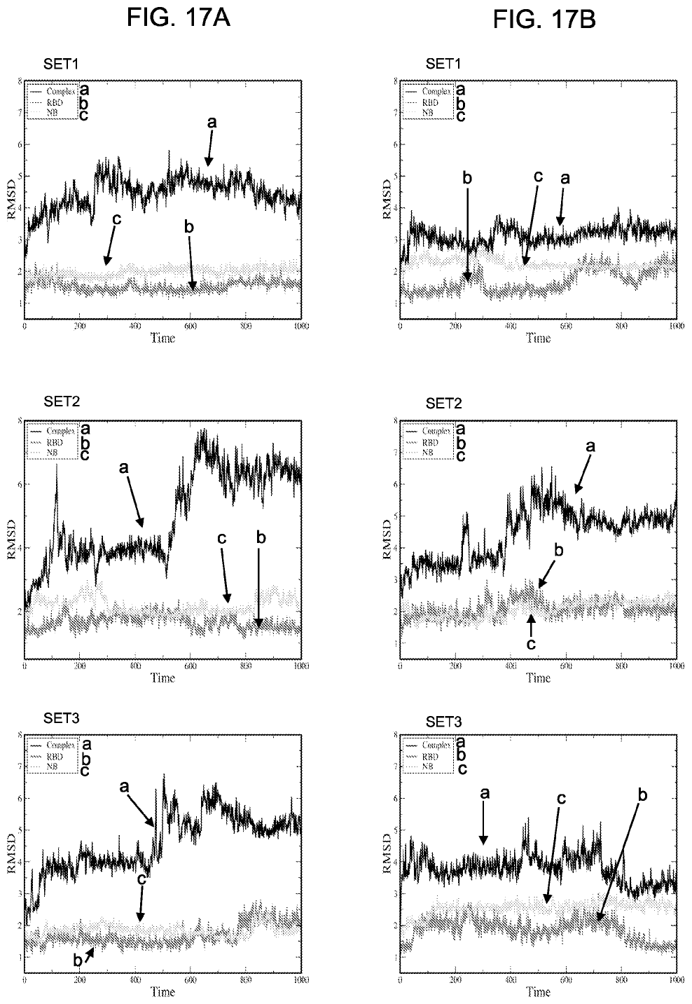

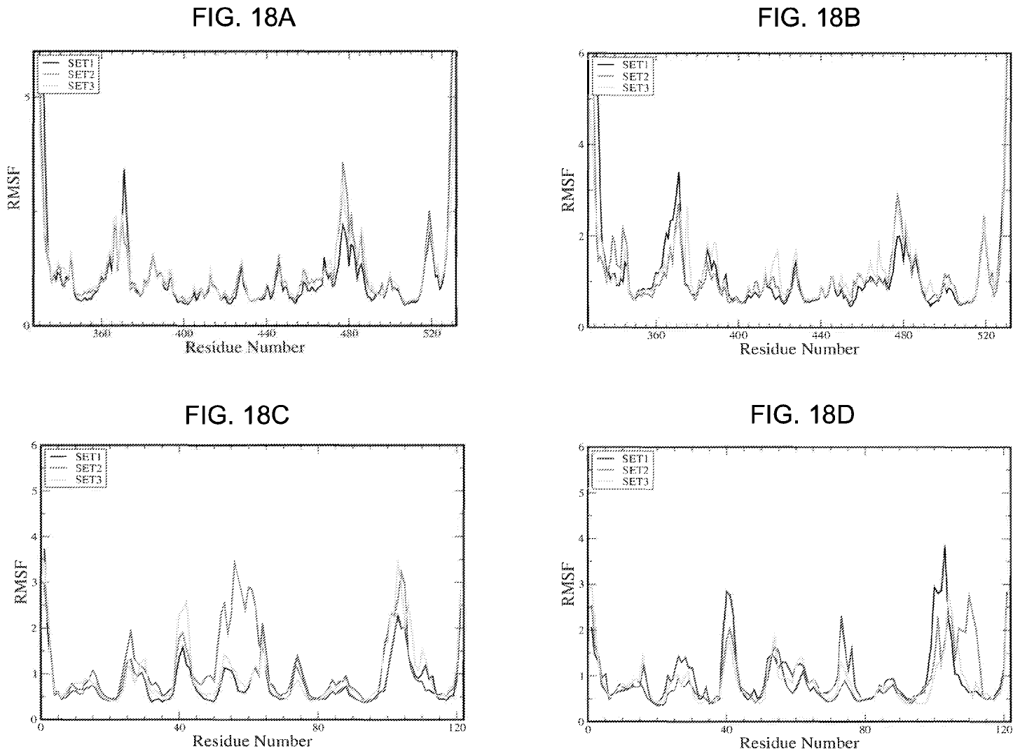

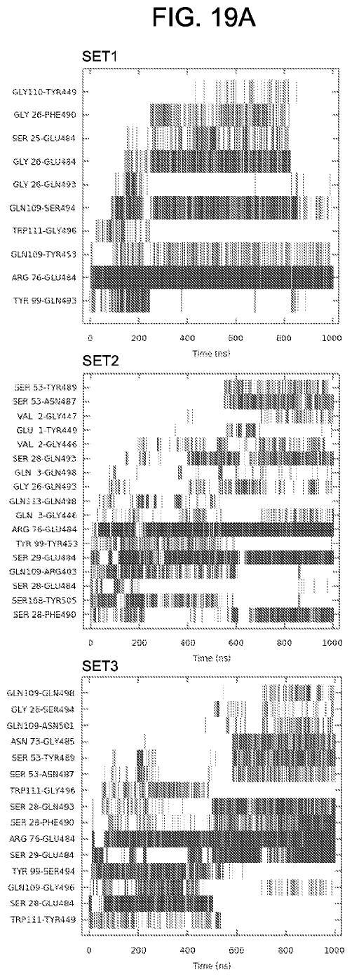

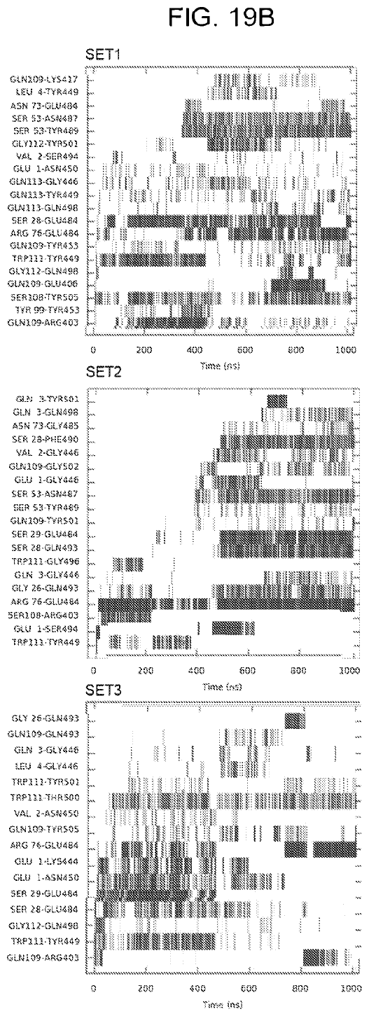

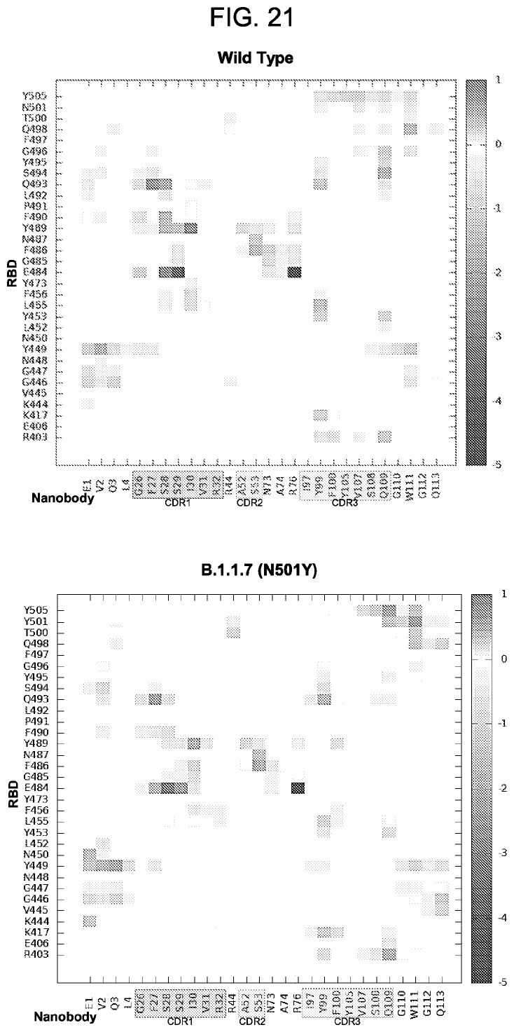

The root mean square deviations of the complexes in all simulations showed that the systems were stable (FIGS. 17A-17B). The consistent pattern of the curve, showing mainly one jump, indicate at least two possible distributions in both systems (FIGS. 17A, SET2 and SET3). The highly conserved pattern of interactions contributes to the very similar binding mode of RBD-1-2G to the WT and B.1.1.7 (Alpha) variant. The MD results showed that RBD-1-2G binds in a similar angle to both the WT and B.1.1.7 (Alpha) variant (FIGS. 5A and 5B). When comparing the position fluctuations per residue in each complex, similar patterns in root mean square fluctuations (RMSFs) (FIGS. 18A-18D), with larger variations in two distinct regions around the RBD residues 355-375 and 470-490 (FIGS. 5A and 5B) were observed. From the analysis of interactions, the salt bridge between E484 (RBD) and R76 (in the constant region of RBD-1-2G) was found to be the most prevalent interaction in the complex containing the WT RBD (FIGS. 5C and 21). The conformation adopted for RBD-1-2G in the WT complex shows that salt bridge between E484 and R76 is further stabilized by the interaction of E484 with S25 (FIG. 5D) or S28, S29, and G26 in some instances. Although this salt bridge remains as one of the most prevalent interactions in the mutant RBD complex, E484 is not dedicated exclusively to this interaction. The residues S28, S29 and N73 in the RBD-1-2G are also involved in interactions with E484 (FIGS. 5D and 5E). An increase in the number of pairs of hydrogen ions or salt bridges was observed in the complex with the mutant RBD compared with the WT complex (FIG. 19). CDR1 and CDR3, as well as a few residues in the constant region (R76 and N73), were responsible for the stabilization of the complex. A few residues in the N-terminal constant region (E1, V2, Q3) and CDR2 (A52 and S53) interact with the RBD in both the WT and mutant forms, whereas only S53 in the CDR2 region seems to be involved with making some hydrogen bonds with N487 and Y489.

The MM/GBSA method was used to estimate the binding free energy (AG binding) for nanobody-RBD complex system. One thousand snapshots were taken at 20-30 ns time intervals throughout the MD simulation trajectory to compute the MM/GBSA free energy difference. As shown in FIG. 5C, the average binding energies of RBD-1-2G/WT RBD was-36.1 kcal/mol, while for the RBD-1-2G/B.1.1.7 RBD was-38.9 kcal/mol. Thus RBD-1-2G can form stable complexes with both the WT and B.1.1.7 variant, with the variant being slightly favored. The predicted interaction energy provided by each residue showed that the salt bridge between E484 and R76 had the most significant contribution to the free energy of binding in both simulations (FIGS. 5C and 21). This residue pair plays an important role in the stabilization of both complexes, which is consistent with the observation of similar binding modes and the activity of these nanobodies against the WT and B.1.1.7 variant.

In view of the many possible implementations to which the principles of the disclosed subject matter may be applied, it should be recognized that the illustrated implementations are only examples of the disclosure and should not be taken as limiting the scope of the disclosure. Rather, the scope of the disclosure is defined by the following claims. We therefore claim all that comes within the scope and spirit of these claims.

1. A method of making a synthetic single-domain monoclonal antibody library, comprising introducing a diversity of nucleic acid molecules encoding complementarity determining region 1 (CDR1), CDR2 and CDR3 sequences between respective framework (FR) coding regions of a synthetic single-domain monoclonal antibody to generate nucleic acid molecules encoding a diversity of synthetic single-domain monoclonal antibodies with the same synthetic single-domain monoclonal antibody scaffold amino acid sequence, wherein the synthetic single-domain monoclonal antibody scaffold comprises a FR1 sequence comprising SEQ ID NO: 1, a FR2 sequence comprising SEQ ID NO: 2, a FR3 sequence comprising SEQ ID NO: 3 and a FR4 sequence comprising SEQ ID NO: 4.

2. The method of claim 1, wherein:
(a) CDR1 is 5 to 8 residues in length and the amino acid residues of the CDR1 sequence are determined according to the following rules:
CDR1 position 1 is G;
CDR1 position 2 is N, S, T or Y;
CDR1 position 3 is I;
CDR1 position 4 is F or S;
CDR1 position 5 is Y, G, D, A, R, S, V, F, L, T, E, P, W, H, K, I, M, N or Q; and
CDR1 positions 6, 7, and/or 8, if present, are individually selected from Y, G, D, A, R, S, V, F, L, T, E, P, W, H, K, I, M, N and Q;
(b) CDR2 is 9 residues in length and the amino acid residues of the CDR2 sequence are determined according to the following rules:
CDR2 position 1 is I;
CDR2 position 2 is A, D, G, N, S or T;
CDR2 position 3 is Y, G, D, A, R, S, V, F, L, T, E, P, W, H, K, I, M, N or Q;
CDR2 position 4 is Y, G, D, A, R, S, V, F, L, T, E, P, W, H, K, I, M, N or Q;
CDR2 position 5 is G;
CDR2 position 6 is A, G, S, or T;
CDR2 position 7 is I, N, S or T;
CDR2 position 8 is T; and
CDR2 position 9 is N or Y; and/or
(c) CDR3 is 14 to 20 amino acids in length and each position is individually selected from Y, G, D, A, R, S, V, F, L, T, E, P, W, H, K, I, M, N and Q.

3-4. (canceled)

5. The method of claim 2, wherein the amino acid composition of each of CDR1 position 5, CDR1 position 6 if present, CDR1 position 7 if present, CDR1 position 8 if present, CDR2 position 3, CDR2 position 4 and each position of CDR3 comprises 13% Y, 12% G, 10% D, 10% A, 8% R, 8% S, 5% V, 5% F, 4% 4% L, 4% T, 3% E, 3% P, 3% W, 2% H, 2% K, 2% 1, 2% M, 2% N and 2% Q.

6. A synthetic single-domain monoclonal antibody library obtainable by the method of claim 1.

7. The synthetic single-domain monoclonal antibody library of claim 6, comprising at least 10¹⁰ unique antibody sequences.

8. A screening method for identifying a synthetic single-domain monoclonal antibody that binds to a target of interest, comprising the use of the synthetic single-domain antibody library of claim 6.

9-11. (canceled)

12. A single-domain monoclonal antibody having the following formula: FR1-CDR1-FR2-CDR2-FR3-CDR3-FR4, wherein:
the amino acid sequence of FR1 comprises SEQ ID NO: 1;
the amino acid sequence of FR2 comprises SEQ ID NO: 2;
the amino acid sequence of FR3 comprises SEQ ID NO: 3; and
the amino acid sequence of FR4 comprises SEQ ID NO: 4.

13. The single-domain monoclonal antibody of claim 12, wherein:
(a) CDR1 is 5 to 8 residues in length and the amino acid residues of the CDR1 sequence are determined according to the following rules:
CDR1 position 1 is G;
CDR1 position 2 is N, S, T or Y;
CDR1 position 3 is I;
CDR1 position 4 is F or S;
CDR1 position 5 is Y, G, D, A, R, S, V, F, L, T, E, P, W, H, K, I, M, N or Q; and
CDR1 positions 6, 7 and/or 8, if present, are randomly selected from Y, G, D, A, R, S, V, F, L, T, E, P, W, H, K, I, M, N or Q;
(b) CDR2 is 9 residues in length and the amino acid residues of the CDR2 sequence are determined according to the following rules:
CDR2 position 1 is I;
CDR2 position 2 is A, D, G, N, S or T;
CDR2 position 3 is Y, G, D, A, R, S, V, F, L, T, E, P, W, H, K, I, M, N or Q;
CDR2 position 4 is Y, G, D, A, R, S, V, F, L, T, E, P, W, H, K, I, M, N or Q;
CDR2 position 5 is G;
CDR2 position 6 is A, G, S, or T;
CDR2 position 7 is I, N, S or T;
CDR2 position 8 is T; and
CDR2 position 9 is N or Y; and/or
(c) CDR3 is 14 to 20 amino acids in length and each position is randomly selected from Y, G, D, A, R, S, V, F, L, T, E, P, W, H, K, I, M, N or Q.

14-15. (canceled)

16. The single-domain monoclonal antibody of claim 12, wherein the amino acid of each of CDR1 position 5, CDR1 position 6 if present, CDR1 position 7 if present, CDR1 position 8 if present, CDR2 position 3, CDR2 position 4 and each position of CDR3 are selected at the following percentages: 13% Y, 12% G, 10% D, 10% A, 8% R, 8% S, 5% V, 5% F, 4% 4% L, 4% T, 3% E, 3% P, 3% W, 2% H, 2% K, 2% I, 2% M, 2% N and 2% Q.

17. A nucleic acid molecule encoding the single-domain monoclonal antibody of claim 12.

18. (canceled)

19. A vector comprising the nucleic acid molecule of claim 17.

20. An isolated host cell comprising the vector of claim 19.

21. A single-domain monoclonal antibody that specifically binds a severe acute respiratory syndrome coronavirus 2 (SARS-COV-2) spike protein, wherein the antibody comprises the complementarity determining region 1 (CDR1), CDR2 and CDR3 sequences of SEQ ID NO: 5.

22. (canceled)

23. The single-domain monoclonal antibody of claim 21, wherein the CDR1, CDR2 and CDR3 sequences respectively comprise residues 26-32, 50-58 and 97-110 of SEQ ID NO: 5.

24. The single-domain monoclonal antibody of claim 21, wherein:
The amino acid sequence is at least 90% identical to SEQ ID NO: 5 and comprises residues 26-32, 50-58 and 97-110 of SEQ ID NO: 5; or
the amino acid sequence of the antibody comprises or consists of SEQ ID NO: 5.

25. The single-domain monoclonal antibody of claim 21, comprising a framework region 1 (FR1), a FR2, a FR3 and a FR4, wherein:
the amino acid sequence of FR1 comprises SEQ ID NO: 1;
the amino acid sequence of FR2 comprises SEQ ID NO: 2;
the amino acid sequence of FR3 comprises SEQ ID NO: 3; and/or
the amino acid sequence of FR4 comprises SEQ ID NO: 4.

26. (canceled)

27. The single-domain monoclonal antibody of claim 21, wherein:
the antibody is a humanized antibody;
the antibody neutralizes SARS-COV-2; and/or
the antibody is conjugated to a detectable label.

28-29. (canceled)

30. A fusion protein comprising the single-domain monoclonal antibody of claim 21 and a heterologous protein.

31. The fusion protein of claim 30, wherein:
the heterologous protein comprises a human Fc protein;
the heterologous protein comprises a human Fc protein comprising a modification that increases half-life of the fusion protein;
the heterologous protein comprises a human Fc protein comprising a modification that increases half-life of the fusion protein, wherein the modification increases binding to the neonatal Fc receptor;
the heterologous protein comprises a protein tag; or
the heterologous protein comprises a protein tag, wherein the amino acid sequence of the protein tag comprises or consists of SEQ ID NO: 6.

32-35. (canceled)

36. A bivalent antibody, a trivalent single-chain Fv, or a bispecific antibody, comprising the single-domain monoclonal antibody of claim 21.

37-38. (canceled)

39. A nucleic acid molecule encoding the single-domain monoclonal antibody of claim 21.

40. (canceled)

41. A vector comprising the nucleic acid molecule of claim 39.

42. A host cell comprising the vector of claim 41.

43. A composition comprising a pharmaceutically acceptable carrier and the single-domain monoclonal antibody of claim 21.

44. A method of producing a single-domain monoclonal antibody that specifically binds to a SARS-COV-2 spike protein, comprising:
expressing a nucleic acid molecule encoding the single-domain monoclonal antibody of claim 21 in a host cell; and
purifying the single-domain monoclonal antibody.

45. A method of detecting the presence of a coronavirus in a biological sample from a subject, comprising:
contacting the biological sample with an effective amount of the single-domain monoclonal antibody of claim 21 under conditions sufficient to form an immune complex; and
detecting the presence of the immune complex in the biological sample, wherein the presence of the immune complex in the biological sample indicates the presence of the coronavirus in the sample.

46. (canceled)

47. A method of inhibiting a coronavirus infection in a subject, comprising administering an effective amount of the single-domain monoclonal antibody of claim 21.

48. The method of claim 47, wherein the coronavirus is SARS-COV-2.

49-50. (canceled)
